SEQUENCE LISTING (55 sequences)

SEQ ID NO: 1 - protein, 353 aa, Pyrococcus horikoshii
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKI

SEQ ID NO: 2 - protein, 420 aa, Synthetic construct
MEVRNMVDYELLKKVVEAPGVSGYEFLGIRDVVIEEIKDYVDEVKVDKLGNVIAHKKGEGPKVMIAAHMDQIGLMVTHIEKNGFLRVAPIGGVDPKTLIAQRFKVWIDKGKFIYGVGASVPPHIQKPEDRKKAPDWDQIFIDIGAESKEEAEDMGVKIGTVITWDGRLERLGKHRFVSIAFDDRIAVYTILEVAKQLKDAKADVYFVATVQEEVGLRGARTSAFGIEPDYGFAIDVTIAADIPGTPEHKQVTHLGKGTAIKIMDRSVICHPTIVRWLEELAKKHEIPYQLEILLGGGTDAGAIHLTKAGVPTGALSVPARYIHSNTEVVDERDVDATVELMTKALENIHELKIGGSHHHHHHHHHHGGGSGGGSGGGSGLNDFFEAQKIEWHEGGGSGGGSGGGSGLNDFFEAQKIEWHE

SEQ ID NO: 3 - protein, 353 aa, Pyrococcus horikoshii
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANMQINREGVATAVLSIPIRYMHSQVELADARDVDNTIKLAKALLEELKPMDFTP

SEQ ID NO: 4 - protein, 364 aa, Synthetic construct
MDLKGGESMVDWKLMQEIIEAPGVSGYEHLGIRDIVVDVLKEVADEVKVDKLGNVIAHFKGSSPRIMVAAHMDKIGVMVNHIDKDGYLHIVPIGGVLPETLVAQRIRFFTEKGERYGVVGVLPPHLRRGQEDKGSKIDWDQIVVDVGASSKEEAEEMGFRVGTVGEFAPNFTRLNEHRFATPYLDDRICLYAMIEAARQLGDHEADIYIVGSVQEEVGLRGARVASYAINPEVGIAMDVTFAKQPHDKGKIVPELGKGPVMDVGPNINPKLRAFADEVAKKYEIPLQVEPSPRPTGTDANMQINREGVATAVLSIPIRYMHSQVELADARDVDNTIKLAKALLEELKPMDFTPGHHHHHHHHHH

SEQ ID NO: 5 - protein, 447 aa, Yersinia pestis
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQLE

SEQ ID NO: 6 - protein, 453 aa, Synthetic construct
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQLEHHHHHH

SEQ ID NO: 7 - protein, 445 aa, Synthetic construct
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQ

SEQ ID NO: 8 - protein, 454 aa, Synthetic construct
MTQQEYQNRRQALLAKMAPGSAAIIFAAPEATRSADSEYPYRQNSDFSYLTGFNEPEAVLILVKSDETHNHSVLFNRIRDLTAEIWFGRRLGQEAAPTKLAVDRALPFDEINEQLYLLLNRLDVIYHAQGQYAYADNIVFAALEKLRHGFRKNLRAPATLTDWRPWLHEMRLFKSAEEIAVLRRAGEISALAHTRAMEKCRPGMFEYQLEGEILHEFTRHGARYPAYNTIVGGGENGCILHYTENECELRDGDLVLIDAGCEYRGYAGDITRTFPVNGKFTPAQRAVYDIVLAAINKSLTLFRPGTSIREVTEEVVRIMVVGLVELGILKGDIEQLIAEQAHRPFFMHGLSHWLGMDVHDVGDYGSSDRGRILEPGMVLTVEPGLYIAPDADVPPQYRGIGIRIEDDIVITATGNENLTASVVKDPDDIEALMALNHAGENLYFQGGSHHHHHH

SEQ ID NO: 9 - protein, 901 aa, Legionella pneumophila
MMVKQGVFMKTDQSKVKKLSDYKSLDYFVIHVDLQIDLSKKPVESKARLTVVPNLNVDSHSNDLVLDGENMTLVSLQMNDNLLKENEYELTKDSLIIKNIPQNTPFTIEMTSLLGENTDLFGLYETEGVALVKAESEGLRRVFYLPDRPDNLATYKTTIIANQEDYPVLLSNGVLIEKKELPLGLHSVTWLDDVPKPSYLFALVAGNLQRSVTYYQTKSGRELPIEFYVPPSATSKCDFAKEVLKEAMAWDERTFNLECALRQHMVAGVDKYASGASEPTGLNLFNTENLFASPETKTDLGILRVLEVVAHEFFHYWSGDRVTIRDWFNLPLKEGLTTFRAAMFREELFGTDLIRLLDGKNLDERAPRQSAYTAVRSLYTAAAYEKSADIFRMMMLFIGKEPFIEAVAKFFKDNDGGAVTLEDFIESISNSSGKDLRSFLSWFTESGIPELIVTDELNPDTKQYFLKIKTVNGRNRPIPILMGLLDSSGAEIVADKLLIVDQEEIEFQFENIQTRPIPSLLRSFSAPVHMKYEYSYQDLLLLMQFDTNLYNRCEAAKQLISALINDFCIGKKIELSPQFFAVYKALLSDNSLNEWMLAELITLPSLEELIENQDKPDFEKLNEGRQLIQNALANELKTDFYNLLFRIQISGDDDKQKLKGFDLKQAGLRRLKSVCFSYLLNVDFEKTKEKLILQFEDALGKNMTETALALSMLCEINCEEADVALEDYYHYWKNDPGAVNNWFSIQALAHSPDVIERVKKLMRHGDFDLSNPNKVYALLGSFIKNPFGFHSVTGEGYQLVADAIFDLDKINPTLAANLTEKFTYWDKYDVNRQAMMISTLKIIYSNATSSDVRTMAKKGLDKVKEDLPLPIHLTFHGGSTMQDRTAQLIADGNKENAYQLH

SEQ ID NO: 10 - protein, 265 aa, Escherichia coli
MGTAISIKTPEDIEKMRVAGRLAAEVLEMIEPYVKPGVSTGELDRICNDYIVNEQHAVSACLGYHGYPKSVCISINEVVCHGIPDDAKLLKDGDIVNIDVTVIKDGFHGDTSKMFIVGKPTIMGERLCRITQESLYLALRMVKPGINLREIGAAIQKFVEAEGFSVVREYCGHGIGRGFHEEPQVLHYDSRETNVVLKPGMTFTIEPMVNAGKKEIRTMKDGWTVKTKDRSLSAQYEHTIVVTDNGCEILTLRKDDTIPAIISHD

SEQ ID NO: 11 - protein, 322 aa, Mycobacterium smegmatis
MGTLEANTNGPGSMLSRMPVSSRTVPFGDHETWVQVTTPENAQPHALPLIVLHGGPGMAHNYVANIAALADETGRTVIHYDQVGCGNSTHLPDAPADFWTPQLFVDEFHAVCTALGIERYHVLGQSWGGMLGAEIAVRQPSGLVSLAICNSPASMRLWSEAAGDLRAQLPAETRAALDRHEAAGTITHPDYLQAAAEFYRRHVCRVVPTPQDFADSVAQMEAEPTVYHTMNGPNEFHVVGTLGDWSVIDRLPDVTAPVLVIAGEHDEATPKTWQPFVDHIPDVRSHVFPGTSHCTHLEKPEEFRAVVAQFLHQHDLAADARV

SEQ ID NO: 12 - protein, 303 aa, Pyrococcus furiosus
MDTEKLMKAGEIAKKVREKAIKLARPGMLLLELAESIEKMIMELGGKPAFPVNLSINEIAAHYTPYKGDTTVLKEGDYLKIDVGVHIDGFIADTAVTVRVGMEEDELMEAAKEALNAAISVARAGVEIKELGKAIENEIRKRGFKPIVNLSGHKIERYKLHAGISIPNIYRPHDNYVLKEGDVFAIEPFATIGAGQVIEVPPTLIYMYVRDVPVRVAQARFLLAKIKREYGTLPFAYRWLQNDMPEGQLKLALKTLEKAGAIYGYPVLKEIRNGIVAQFEHTIIVEKDSVIVTQDMINKSTLE

SEQ ID NO: 13 - protein, 428 aa, Aeromonas sobria
HMSSPLHYVLDGIHCEPHFFTVPLDHQQPDDEETITLFGRTLCRKDRLDDELPWLLYLQGGPGFGAPRPSANGGWIKRALQEFRVLLLDQRGTGHSTPIHAELLAHLNPRQQADYLSHFRADSIVRDAELIREQLSPDHPWSLLGQSFGGFCSLTYLSLFPDSLHEVYLTGGVAPIGRSADEVYRATYQRVADKNRAFFARFPHAQAIANRLATHLQRHDVRLPNGQRLTVEQLQQQGLDLGASGAFEELYYLLEDAFIGEKLNPAFLYQVQAMQPFNTNPVFAILHELIYCEGAASHWAAERVRGEFPALAWAQGKDFAFTGEMIFPWMFEQFRELIPLKEAAHLLAEKADWGPLYDPVQLARNKVPVACAVYAEDMYVEFDYSRETLKGLSNSRAWITNEYEHNGLRVDGEQILDRLIRLNRDCLE

SEQ ID NO: 14 - protein, 348 aa, Pyrococcus furiosus
MKERLEKLVKFMDENSIDRVFIAKPVNVYYFSGTSPLGGGYIIVDGDEATLYVPELEYEMAKEESKLPVVKFKKFDEIYEILKNTETLGIEGTLSYSMVENFKEKSNVKEFKKIDDVIKDLRIIKTKEEIEIIEKACEIADKAVMAAIEEITEGKREREVAAKVEYLMKMNGAEKPAFDTIIASGHRSALPHGVASDKRIERGDLVVIDLGALYNHYNSDITRTIVVGSPNEKQREIYEIVLEAQKRAVEAAKPGMTAKELDSIAREIIKEYGYGDYFIHSLGHGVGLEIHEWPRISQYDETVLKEGMVITIEPGIYIPKLGGVRIEDTVLITENGAKRLTKTERELL

SEQ ID NO: 15 - protein, 298 aa, Elizabethkingia meningoseptica
MIPITTPVGNFKVWTKRFGTNPKIKVLLLHGGPAMTHEYMECFETFFQREGFEFYEYDQLGSYYSDQPTDEKLWNIDRFVDEVEQVRKAIHADKENFYVLGNSWGGILAMEYALKYQQNLKGLIVANMMASAPEYVKYAEVLSKQMKPEVLAEVRAIEAKKDYANPRYTELLFPNYYAQHICRLKEWPDALNRSLKHVNSTVYTLMQGPSELGMSSDARLAKWDIKNRLHEIATPTLMIGARYDTMDPKAMEEQSKLVQKGRYLYCPNGSHLAMWDDQKVFMDGVIKFIKDVDTKSFN

SEQ ID NO: 16 - protein, 310 aa, Neisseria gonorrhoeae
MYEIKQPFHSGYLQVSEIHQIYWEESGNPDGVPVIFLHGGPGAGASPECRGFFNPDVFRIVIIDQRGCGRSHPYACAEDNTTWDLVADIEKVREMLGIGKWLVFGGSWGSTLSLAYAQTHPERVKGLVLRGIFLCRPSETAWLNEAGGVSRIYPEQWQKFVAPIAENRRNRLIEAYHGLLFHQDEEVCLSAAKAWADWESYLIRFEPEGVDEDAYASLAIARLENHYFVNGGWLQGDKAILNNIGKIRHIPTVIVQGRYDLCTPMQSAWELSKAFPEAELRVVQAGHCAFDPPLADALVQAVEDILPRLL

SEQ ID NO: 17 - protein, 870 aa, Escherichia coli
MTQQPQAKYRHDYRAPDYQITDIDLTFDLDAQKTVVTAVSQAVRHGASDAPLRLNGEDLKLVSVHINDEPWTAWKEEEGALVISNLPERFTLKIINEISPAANTALEGLYQSGDALCTQCEAEGFRHITYYLDRPDVLARFTTKIIADKIKYPFLLSNGNRVAQGELENGRHWVQWQDPFPKPCYLFALVAGDFDVLRDTFTTRSGREVALELYVDRGNLDRAPWAMTSLKNSMKWDEERFGLEYDLDIYMIVAVDFFNMGAMENKGLNIFNSKYVLARTDTATDKDYLDIERVIGHEYFHNWTGNRVTCRDWFQLSLKEGLTVFRDQEFSSDLGSRAVNRINNVRTMRGLQFAEDASPMAHPIRPDMVIEMNNFYTLTVYEKGAEVIRMIHTLLGEENFQKGMQLYFERHDGSAATCDDFVQAMEDASNVDLSHFRRWYSQSGTPIVTVKDDYNPETEQYTLTISQRTPATPDQAEKQPLHIPFAIELYDNEGKVIPLQKGGHPVNSVLNVTQAEQTFVFDNVYFQPVPALLCEFSAPVKLEYKWSDQQLTFLMRHARNDFSRWDAAQSLLATYIKLNVARHQQGQPLSLPVHVADAFRAVLLDEKIDPALAAEILTLPSVNEMAELFDIIDPIAIAEVREALTRTLATELADELLAIYNANYQSEYRVEHEDIAKRTLRNACLRFLAFGETHLADVLVSKQFHEANNMTDALAALSAAVAAQLPCRDALMQEYDDKWHQNGLVMDKWFILQATSPAANVLETVRGLLQHRSFTMSNPNRIRSLIGAFAGSNPAAFHAEDGSGYLFLVEMLTDLNSRNPQVASRLIEPLIRLKRYDAKRQEKMRAALEQLKGLENLSGDLYEKITKALA

SEQ ID NO: 18 - protein, 889 aa, unidentified
  source 1..889: note = Plasmodium falciparum
PKIHYRKDYKPSGFIINQVTLNINIHDQETIVRSVLDMDISKHNVGEDLVFDGVGLKINEISINNKKLVEGEEYTYDNEFLTIFSKFVPKSKFAFSSEVIIHPETNYALTGLYKSKNIIVSQCEATGFRRITFFIDRPDMMAKYDVTVTADKEKYPVLLSNGDKVNEFEIPGGRHGARFNDPPLKPCYLFAVVAGDLKHLSATYITKYTKKKVELYVFSEEKYVSKLQWALECLKKSMAFDEDYFGLEYDLSRLNLVAVSDFNVGAMENKGLNIFNANSLLASKKNSIDFSYARILTVVGHEYFHQYTGNRVTLRDWFQLTLKEGLTVHRENLFSEEMTKTVTTRLSHVDLLRSVQFLEDSSPLSHPIRPESYVSMENFYTTTVYDKGSEVMRMYLTILGEEYYKKGFDIYIKKNDGNTATCEDFNYAMEQAYKMKKADNSANLNQYLLWFSQSGTPHVSFKYNYDAEKKQYSIHVNQYTKPDENQKEKKPLFIPISVGLINPENGKEMISQTTLELTKESDTFVFNNIAVKPIPSLFRGFSAPVYIEDQLTDEERILLLKYDSDAFVRYNSCTNIYMKQILMNYNEFLKAKNEKLESFQLTPVNAQFIDAIKYLLEDPHADAGFKSYIVSLPQDRYIINFVSNLDTDVLADTKEYIYKQIGDKLNDVYYKMFKSLEAKADDLTYFNDESHVDFDQMNMRTLRNTLLSLLSKAQYPNILNEIIEHSKSPYPSNWLTSLSVSAYFDKYFELYDKTYKLSKDDELLLQEWLKTVSRSDRKDIYEILKKLENEVLKDSKNPNDIRAVYLPFTNNLRRFHDISGKGYKLIAEVITKTDKFNPMVATQLCEPFKLWNKLDTKRQELMLNEMNTMLQEPQISNNLKEYLLRLTNK

SEQ ID NO: 19 - protein, 919 aa, Synthetic construct
MWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHELAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 20 - protein, 919 aa, Synthetic construct
MWLAAAAPSLARRLLFLGPPPPPLLLLVFSRSSRRRLHSLGLAAMPEKRPFERLPADVSPINYSLCLKPDLLDFTFEGKLEAAAQVRQATNQIVMNCADIDIITASYAPEGDEEIHATGFNYQNEDEKVTLSFPSTLQTGTGTLKIDFVGELNDKMKGFYRSKYTTPSGEVRYAAVTQFEATDARRAFPCWDEPAIKATFDISLVVPKDRVALSNMNVIDRKPYPDDENLVEVKFARTPVMSTYLVAFVVGEYDFVETRSKDGVCVRVYTPVGKAEQGKFALEVAAKTLPFYKDYFNVPYPLPKIDLIAIADFAAGAMENWGLVTYRETALLIDPKNSCSSSRQWVALVVGHVLAHQWFGNLVTMEWWTHLWLNEGFASWIEYLCVDHCFPEYDIWTQFVSADYTRAQELDALDNSHPIEVSVGHPSEVDEIFDAISYSKGASVIRMLHDYIGDKDFKKGMNMYLTKFQQKNAATEDLWESLENASGKPIAAVMNTWTKQMGFPLIYVEAEQVEDDRLLRLSQKKFCAGGSYVGEDCPQWMVPITISTSEDPNQAKLKILMDKPEMNVVLKNVKPDQWVKLNLGTVGFYRTQYSSAMLESLLPGIRDLSLPPVDRLGLQNDLFSLARAGIISTVEVLKVMEAFVNEPNYTVWSDLSCNLGILSTLLSHTDFYEEIQEFVKDVFSPIGERLGWDPKPGEGHLDALLRGLVLGKLGKAGHKATLEEARRRFKDHVEGKQILSADLRSPVYLTVLKHGDGTTLDIMLKLHKQADMQEEKNRIERVLGATLLPDLIQKVLTFALSEEVRPQDTVSVIGGVAGGSKHGRKAAWKFIKDNWEELYNRYQGGFLISRLIKLSVEGFAVDKMAGEVKAFFESHPAPSAERTIQQCCENILLNAAWLKRDAESIHQYLLQRKASPPTV

SEQ ID NO: 21 - protein, 864 aa, Francisella tularensis
MIYEFVMTDPKIKYLKDYKPSNYLIDETHLIFELDESKTRVTANLYIVANRENRENNTLVLDGVELKLLSIKLNNKHLSPAEFAVNENQLIINNVPEKFVLQTVVEINPSANTSLEGLYKSGDVFSTQCEATGFRKITYYLDRPDVMAAFTVKIIADKKKYPIILSNGDKIDSGDISDNQHFAVWKDPFKKPCYLFALVAGDLASIKDTYITKSQRKVSLEIYAFKQDIDKCHYAMQAVKDSMKWDEDRFGLEYDLDTFMIVAVPDFNAGAMENKGLNIFNTKYIMASNKTATDKDFELVQSVVGHEYFHNWTGDRVTCRDWFQLSLKEGLTVFRDQEFTSDLNSRDVKRIDDVRIIRSAQFAEDASPMSHPIRPESYIEMNNFYTVTVYNKGAEIIRMIHTLLGEEGFQKGMKLYFERHDGQAVTCDDFVNAMADANNRDFSLFKRWYAQSGTPNIKVSENYDASSQTYSLTLEQTTLPTADQKEKQALHIPVKMGLINPEGKNIAEQVIELKEQKQTYTFENIAAKPVASLFRDFSAPVKVEHKRSEKDLLHIVKYDNNAFNRWDSLQQIATNIILNNADLNDEFLNAFKSILHDKDLDKALISNALLIPIESTIAEAMRVIMVDDIVLSRKNVVNQLADKLKDDWLAVYQQCNDNKPYSLSAEQIAKRKLKGVCLSYLMNASDQKVGTDLAQQLFDNADNMTDQQTAFTELLKSNDKQVRDNAINEFYNRWRHEDLVVNKWLLSQAQISHESALDIVKGLVNHPAYNPKNPNKVYSLIGGFGANFLQYHCKDGLGYAFMADTVLALDKFNHQVAARMARNLMSWKRYDSDRQAMMKNALEKIKASNPSKNVFEIVSKSLES

SEQ ID NO: 22 - protein, 408 aa, Thermus aquaticus
MDAFTENLNKLAELAIRVGLNLEEGQEIVATAPIEAVDFVRLLAEKAYENGASLFTVLYGDNLIARKRLALVPEAHLDRAPAWLYEGMAKAFHEGAARLAVSGNDPKALEGLPPERVGRAQQAQSRAYRPTLSAITEFVTNWTIVPFAHPGWAKAVFPGLPEEEAVQRLWQAIFQATRVDQEDPVAAWEAHNRVLHAKVAFLNEKRFHALHFQGPGTDLTVGLAEGHLWQGGATPTKKGRLCNPNLPTEEVFTAPHRERVEGVVRASRPLALSGQLVEGLWARFEGGVAVEVGAEKGEEVLKKLLDTDEGARRLGEVALVPADNPIAKTGLVFFDTLFDENAASHIAFGQAYAENLEGRPSGEEFRRRGGNESMVHVDWMIGSEEVDVDGLLEDGTRVPLMRRGRWVI

SEQ ID NO: 23 - protein, 362 aa, unidentified
  source 1..362: note = Bacillus stearothermophilus
MAKLDETLTMLKALTDAKGVPGNEREARDVMKTYIAPYADEVTTDGLGSLIAKKEGKSGGPKVMIAGHLDEVGFMVTQIDDKGFIRFQTLGGWWSQVMLAQRVTIVTKKGDITGVIGSKPPHILPSEARKKPVEIKDMFIDIGATSREEAMEWGVRPGDMIVPYFEFTVLNNEKMLLAKAWDNRIGCAVAIDVLKQLKGVDHPNTVYGVGTVQEEVGLRGARTAAQFIQPDIAFAVDVGIAGDTPGVSEKEAMGKLGAGPHIVLYDATMVSHRGLREFVIEVAEELNIPHHFDAMPGVGTDAGAIHLTGIGVPSLTIAIPTRYIHSHAAILHRDDYENTVKLLVEVIKRLDADKVKQLTFDE

SEQ ID NO: 24 - protein, 484 aa, unidentified
  source 1..484: note = Vibrio cholera
MEDKVWISMGADAVGSLNPALSESLLPHSFASGSQVWIGEVAIDELAELSHTMHEQHNRCGGYMVHTSAQGAMAALMMPESIANFTIPAPSQQDLVNAWLPQVSADQITNTIRALSSFNNRFYTTTSGAQASDWLANEWRSLISSLPGSRIEQIKHSGYNQKSVVLTIQGSEKPDEWVIVGGHLDSTLGSHTNEQSIAPGADDDASGIASLSEIIRVLRDNNFRPKRSVALMAYAAEEVGLRGSQDLANQYKAQGKKVVSVLQLDMTNYRGSAEDIVFITDYTDSNLTQFLTTLIDEYLPELTYGYDRCGYACSDHASWHKAGFSAAMPFESKFKDYNPKIHTSQDTLANSDPTGNHAVKFTKLGLAYVIEMANAGSSQVPDDSVLQDGTAKINLSGARGTQKRFTFELSQSKPLTIQTYGGSGDVDLYVKYGSAPSKSNWDCRPYQNGNRETCSFNNAQPGIYHVMLDGYTNYNDVALKASTQ

SEQ ID NO: 25 - protein, 488 aa, Photobacterium halotolerans
MEDKVWISIGSDASQTVKSVMQSNARSLLPESLASNGPVWVGQVDYSQLAELSHHMHEDHQRCGGYMVHSSPESAIAASNMPQSLVAFSIPEISQQDTVNAWLPQVNSQAITGTITSLTSFINRFYTTTSGAQASDWLANEWRSLSASLPNASVRQVSHFGYNQKSVVLTITGSEKPDEWIVLGGHLDSTIGSHTNEQSVAPGADDDASGIASVTEIIRVLSENNFQPKRSIAFMAYAAEEVGLRGSQDLANQYKAEGKQVISALQLDMTNYKGSVEDIVFITDYTDSNLTTFLSQLVDEYLPSLTYGFDTCGYACSDHASWHKAGFSAAMPFEAKFNDYNPMIHTPNDTLQNSDPTASHAVKFTKLGLAYAIEMASTTGGTPPPTGNVLKDGVPVNGLSGATGSQVHYSFELPAQKNLQISTAGGSGDVDLYVSFGSEATKQNWDCRPYRNGNNEVCTFAGATPGTYSIMLDGYRQFSGVTLKASTQ

SEQ ID NO: 26 - protein, 871 aa, Yersinia pestis
MTQQPQAKYRHDYRAPDYTITDIDLDFALDAQKTTVTAVSKVKRQGTDVTPLILNGEDLTLISVSVDGQAWPHYRQQDNTLVIEQLPADFTLTIVNDIHPATNSALEGLYLSGEALCTQCEAEGFRHITYYLDRPDVLARFTTRIVADKSRYPYLLSNGNRVGQGELDDGRHWVKWEDPFPKPSYLFALVAGDFDVLQDKFITRSGREVALEIFVDRGNLDRADWAMTSLKNSMKWDETRFGLEYDLDIYMIVAVDFFNMGAMENKGLNVFNSKYVLAKAETATDKDYLNIEAVIGHEYFHNWTGNRVTCRDWFQLSLKEGLTVFRDQEFSSDLGSRSVNRIENVRVMRAAQFAEDASPMAHAIRPDKVIEMNNFYTLTVYEKGSEVIRMMHTLLGEQQFQAGMRLYFERHDGSAATCDDFVQAMEDVSNVDLSLFRRWYSQSGTPLLTVHDDYDVEKQQYHLFVSQKTLPTADQPEKLPLHIPLDIELYDSKGNVIPLQHNGLPVHHVLNVTEAEQTFTFDNVAQKPIPSLLREFSAPVKLDYPYSDQQLTFLMQHARNEFSRWDAAQSLLATYIKLNVAKYQQQQPLSLPAHVADAFRAILLDEHLDPALAAQILTLPSENEMAELFTTIDPQAISTVHEAITRCLAQELSDELLAVYVANMTPVYRIEHGDIAKRALRNTCLNYLAFGDEEFANKLVSLQYHQADNMTDSLAALAAAVAAQLPCRDELLAAFDVRWNHDGLVMDKWFALQATSPAANVLVQVRTLLKHPAFSLSNPNRTRSLIGSFASGNPAAFHAADGSGYQFLVEILSDLNTRNPQVAARLIEPLIRLKRYDAGRQALMRKALEQLKTLDNLSGDLYEKITKALAA

SEQ ID NO: 27 - protein, 483 aa, Vibrio anguillarum
MEEKVWISIGGDATQTALRSGAQSLLPENLINQTSVWVGQVPVSELATLSHEMHENHQRCGGYMVHPSAQSAMSVSAMPLNLNAFSAPEITQQTTVNAWLPSVSAQQITSTITTLTQFKNRFYTTSTGAQASNWIADHWRSLSASLPASKVEQITHSGYNQKSVMLTITGSEKPDEWVVIGGHLDSTLGSRTNESSIAPGADDDASGIAGVTEIIRLLSEQNFRPKRSIAFMAYAAEEVGLRGSQDLANRFKAEGKKVMSVMQLDMTNYQGSREDIVFITDYTDSNFTQYLTQLLDEYLPSLTYGFDTCGYACSDHASWHAVGYPAAMPFESKFNDYNPNIHSPQDTLQNSDPTGFHAVKFTKLGLAYVVEMGNASTPPTPSNQLKNGVPVNGLSASRNSKTWYQFELQEAGNLSIVLSGGSGDADLYVKYQTDADLQQYDCRPYRSGNNETCQFSNAQPGRYSILLHGYNNYSNASLVANAQ

SEQ ID NO: 28 - protein, 482 aa, Salinivibrio sp.
MEDKKVWISIGADAQQTALSSGAQPLLAQSVAHNGQAWIGEVSESELAALSHEMHENHHRCGGYIVHSSAQSAMAASNMPLSRASFIAPAISQQALVTPWISQIDSALIVNTIDRLTDFPNRFYTTTSGAQASDWIKQRWQSLSAGLAGASVTQISHSGYNQASVMLTIEGSESPDEWVVVGGHLDSTIGSRTNEQSIAPGADDDASGIAAVTEVIRVLAQNNFQPKRSIAFVAYAAEEVGLRGSQDVANQFKQAGKDVRGVLQLDMTNYQGSAEDIVFITDYTDNQLTQYLTQLLDEYLPTLNYGFDTCGYACSDHASWHQVGYPAAMPFEAKFNDYNPNIHTPQDTLANSDSEGAHAAKFTKLGLAYTVELANADSSPNPGNELKLGEPINGLSGARGNEKYFNYRLDQSGELVIRTYGGSGDVDLYVKANGDVSTGNWDCRPYRSGNDEVCRFDNATPGNYAVMLRGYRTYDNVSLIVE

SEQ ID NO: 29 - protein, 301 aa, Vibrio proteolyticus
MPPITQQATVTAWLPQVDASQITGTISSLESFTNRFYTTTSGAQASDWIASEWQALSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLE

SEQ ID NO: 30 - protein, 301 aa, Vibrio proteolyticus
MPPITQQATVTAWLPQVDASQITGTISSLESFTNRFYTTTSGAQASDWIASEWQFLSASLPNASVKQVSHSGYNQKSVVMTITGSEAPDEWIVIGGHLDSTIGSHTNEQSVAPGADDDASGIAAVTEVIRVLSENNFQPKRSIAFMAYAAEEVGLRGSQDLANQYKSEGKNVVSALQLDMTNYKGSAQDVVFITDYTDSNFTQYLTQLMDEYLPSLTYGFDTCGYACSDHASWHNAGYPAAMPFESKFNDYNPRIHTTQDTLANSDPTGSHAKKFTQLGLAYAIEMGSATGDTPTPGNQLE

SEQ ID NO: 31 - protein, 348 aa, Pyrococcus furiosus
MVDWELMKKIIESPGVSGYEHLGIRDLVVDILKDVADEVKIDKLGNVIAHFKGSAPKVMVAAHMDKIGLMVNHIDKDGYLRVVPIGGVLPETLIAQKIRFFTEKGERYGVVGVLPPHLRREAKDQGGKIDWDSIIVDVGASSREEAEEMGFRIGTIGEFAPNFTRLSEHRFATPYLDDRICLYAMIEAARQLGEHEADIYIVASVQEEIGLRGARVASFAIDPEVGIAMDVTFAKQPNDKGKIVPELGKGPVMDVGPNINPKLRQFADEVAKKYEIPLQVEPSPRPTGTDANVMQINREGVATAVLSIPIRYMHSQVELADARDVDNTIKLAKALLEELKPMDFTPLE

SEQ ID NO: 32 - protein, 6 aa, Synthetic construct
HHHHHH

SEQ ID NO: 33 - protein, 9 aa, Synthetic construct
GGSHHHHHH

SEQ ID NO: 34 - protein, 10 aa, Synthetic construct
HHHHHHHHHH

SEQ ID NO: 35 - protein, 11 aa, Synthetic construct
GHHHHHHHHHH

SEQ ID NO: 36 - protein, 15 aa, Synthetic construct
GLNDFFEAQKIEWHE

SEQ ID NO: 37 - protein, 19 aa, Synthetic construct
GGGSGLNDFFEAQKIEWHE

SEQ ID NO: 38 - protein, 27 aa, Synthetic construct
GGGSGGGSGGGSGLNDFFEAQKIEWHE

SEQ ID NO: 39 - protein, 54 aa, Synthetic construct
GGGSGGGSGGGSGLNDFFEAQKIEWHEGGGSGGGSGGGSGLNDFFEAQKIEWHE

SEQ ID NO: 40 - protein, 56 aa, Synthetic construct
GSGGGSGGGSGGGSGLNDFFEAQKIEWHEGGGSGGGSGGGSGLNDFFEAQKIEWHE

SEQ ID NO: 41 - protein, 38 aa, Synthetic construct
GHHHHHHHHHHGGGSGGGSGGGSGLNDFFEAQKIEWHE

SEQ ID NO: 42 - protein, 65 aa, Synthetic construct
GHHHHHHHHHHGGGSGGGSGGGSGLNDFFEAQKIEWHEGGGSGGGSGGGSGLNDFFEAQKIEWHE

SEQ ID NO: 43 - protein, 67 aa, Synthetic construct
GGSHHHHHHHHHHGGGSGGGSGGGSGLNDFFEAQKIEWHEGGGSGGGSGGGSGLNDFFEAQKIEWHE

SEQ ID NO: 44 - protein, 66 aa, Synthetic construct
GSHHHHHHHHHHGGGSGGGSGGGSGLNDFFEAQKIEWHEGGGSGGGSGGGSGLNDFFEAQKIEWHE

SEQ ID NO: 45 - protein, 61 aa, Synthetic construct
GGGSGGGSGGGSGLNDFFEAQKIEWHEGGGSGGGSGGGSGLNDFFEAQKIEWHEGHHHHHH

SEQ ID NO: 46 - protein, 9 aa, Synthetic construct
FAAAYPDDD

SEQ ID NO: 47 - protein, 10 aa, Synthetic construct
RLIFAYPDDD

SEQ ID NO: 48 - protein, 18 aa, Synthetic construct
LASSIAEANRFADIADYP

SEQ ID NO: 49 - protein, 13 aa, Synthetic construct
DQQRLIFAYPDDD

SEQ ID NO: 50 - protein, 22 aa, Synthetic construct
DQQIASSRLAASFAAQQYPDDD

SEQ ID NO: 51 - protein, 15 aa, Synthetic construct
RLAYPAFAAYPDDDF

SEQ ID NO: 52 - protein, 12 aa, Synthetic construct
FPARAFAYPDDD

SEQ ID NO: 53 - protein, 15 aa, Synthetic construct
LAAYPARLAYPDDDF

SEQ ID NO: 54 - protein, 11 aa, Synthetic construct
RLIFAYPDDDK

SEQ ID NO: 55 - protein, 16 aa, Synthetic construct
RLAYPAFAAYPDDDFK

(19) United States
(12) Patent Application Publication
(10) Pub. No.: US 2024/0295562 A1
(43) Pub. Date: Sep. 5, 2024

(54) POLYPEPTIDE CLEAVING REAGENTS AND USES THEREOF

(71) Applicant: Quantum-Si Incorporated, Branford, CT (US)

(72) Inventors: Brian Reed, Madison, CT (US); Manjula Pandey, Guilford, CT (US)

(73) Assignee: Quantum-Si Incorporated, Branford, CT (US)

(21) Appl. No.: 18/491,719

(22) Filed: Oct. 20, 2023

Related U.S. Application Data
(60) Provisional application No. 63/418,287, filed on Oct. 21, 2022.

(51) Int. Cl. G01N 33/68; C12N 9/48

(52) U.S. Cl. CPC G01N 33/6821; C12N 9/485; C12Y 304/11001; G01N 2333/948

(57) ABSTRACT

There is provided amino acid cleaving reagents with improved cleavage activity, allowing for more structural information to be obtained from polypeptides in sequencing reactions.

### CROSS-REFERENCE TO RELATED APPLICATIONS

This application claims priority under 35 U.S.C. § 119(e) to U.S. Provisional Patent Application No. 63/418,287, filed Oct. 21, 2022, which is hereby incorporated by reference in its entirety.

### REFERENCE TO AN ELECTRONIC SEQUENCE LISTING

The contents of the electronic sequence listing (R070870165US01-SEQ-JIB.xml; Size: 64,872 bytes; and Date of Creation: Oct. 20, 2023) is herein incorporated by reference in its entirety.

### BACKGROUND

Proteins are the main structural and functional components of cells, driving key biological and cellular processes. Next-generation DNA sequencing technologies have revolutionized our understanding of heredity and gene regulation, but the complex and dynamic states of cells are not fully captured by the genome and transcriptome. Applying similar approaches to proteomics has been difficult because of the scale, dynamic range, and inability to amplify the source.

### SUMMARY

In some aspects, the disclosure provides a composition comprising: a first cleaving reagent comprising a first aminopeptidase from Pyrococcus horikoshii and a first tag sequence; and a second cleaving reagent comprising a second aminopeptidase from Pyrococcus horikoshii, where the first and second aminopeptidases have different amino acid sequences.

In some embodiments, the amino acid sequences of the first and second aminopeptidases share less than 80% sequence identity (e.g., less than 70%, less than 60%, less than 50%, 10-80%, 20-60%, 30-50%, or 40-50% sequence identity). In some embodiments, the first aminopeptidase has an amino acid sequence that is at least 80% identical to SEQ ID NO: 3. In some embodiments, the second aminopeptidase has an amino acid sequence that is at least 80% identical to SEQ ID NO: 1.

In some embodiments, the second cleaving reagent comprises a second tag sequence. In some embodiments, the composition further comprises a third cleaving reagent comprising an aminopeptidase from Yersinia pestis.

In some aspects, the disclosure provides a composition comprising: a first cleaving reagent comprising a first aminopeptidase having an amino acid sequence that is at least 80% identical to SEQ ID NO: 3; a second cleaving reagent comprising a second aminopeptidase having an amino acid sequence that is at least 80% identical to SEQ ID NO: 1; and a third cleaving reagent comprising a third aminopeptidase having an amino acid sequence that is at least 80% identical to SEQ ID NO: 5 or 7.

In some embodiments, the first cleaving reagent comprises a first tag sequence; the second cleaving reagent comprises a second tag sequence; and the third cleaving reagent comprises a third tag sequence. In some embodiments, the first tag sequence is attached to a terminal end of the first aminopeptidase; the second tag sequence is attached to a terminal end of the second aminopeptidase; and the third tag sequence is attached to a terminal end of the third aminopeptidase. In some embodiments, each tag sequence is attached to the C-terminus of its respective aminopeptidase. In some embodiments, each tag sequence independently comprises at least two amino acids (e.g., 2-200, 2-100, 4-80, 5-50, 5-30, 5-20, 10-100, 20-80, 30-70 amino acids).

In some embodiments, at least one of the first, second, and third tag sequences comprises a polyhistidine-tag. In some embodiments, at least one of the first, second, and third tag sequences comprises a biotinylation tag. In some embodiments, the biotinylation tag comprises at least one biotin ligase recognition sequence. In some embodiments, the biotinylation tag comprises two biotin ligase recognition sequences oriented in tandem.

In some embodiments, the first, second, and third cleaving reagents are present in the composition at a first, second, and third concentration, respectively, where the first concentration is at least two-fold higher than the second concentration. In some embodiments, the first concentration is at least five-fold higher than the third concentration. In some embodiments, the second concentration is at least two-fold higher than the third concentration. In some embodiments, the molar ratio of the first cleaving reagent to the second cleaving reagent in the composition is between about 2:1 and about 20:1. In some embodiments, the molar ratio of the first cleaving reagent to the third cleaving reagent in the composition is between about 5:1 and about 200:1.

In some embodiments, the first cleaving reagent is a polypeptide having an amino acid sequence that is at least 80% identical to SEQ ID NO: 4. In some embodiments, the second cleaving reagent is a polypeptide having an amino acid sequence that is at least 80% identical to SEQ ID NO: 2. In some embodiments, the third cleaving reagent is a polypeptide having an amino acid sequence that is at least 80% identical to SEQ ID NO: 8.

In some aspects, the disclosure provides reaction mixtures for polypeptide analysis. In some embodiments, a reaction mixture comprises: a composition described herein; and one or more amino acid binding proteins not having peptide cleavage activity. In some embodiments, a reaction mixture comprises: a cleaving reagent comprising a first aminopeptidase having an amino acid sequence that is at least 92% (e.g., 92-99%, 94-100%, 96-100%, 98-100%) identical to SEQ ID NO: 3 and comprising a first tag sequence; and one or more amino acid binding proteins not having peptide cleavage activity.

In some aspects, the disclosure provides methods of polypeptide analysis. In some embodiments, a method of polypeptide analysis comprises: contacting a polypeptide with a reaction mixture described herein; monitoring a signal for signal pulses corresponding to interactions between one or more amino acid binding proteins and the polypeptide; and determining at least one chemical characteristic of the polypeptide based on a characteristic pattern in the signal.

In some aspects, the disclosure provides a system comprising: at least one hardware processor; and at least one non-transitory computer-readable storage medium storing processor-executable instructions that, when executed by the at least one hardware processor, cause the at least one hardware processor to perform a method described herein. In some aspects, the disclosure provides at least one non-transitory computer-readable storage medium storing processor-executable instructions that, when executed by at least one hardware processor, cause the at least one hardware processor to perform a method described herein.

The details of certain embodiments of the disclosure are set forth in the Detailed Description. Other features, objects, and advantages of the disclosure will be apparent from the Examples, Drawings, and Claims.

### BRIEF DESCRIPTION OF THE DRAWINGS

The accompanying Drawings, which constitute a part of this specification, illustrate several embodiments of the disclosure and together with the accompanying description, serve to explain the principles of the disclosure.

FIG. 1A shows an example overview of real-time dynamic protein sequencing. Protein samples are digested into peptide fragments, immobilized in nanoscale reaction chambers, and incubated with a mixture of freely-diffusing N-terminal amino acid (NAA) recognizers and aminopeptidases that carry out the sequencing process. The labeled recognizers bind on and off to the peptide when one of their cognate NAAs is exposed at the N-terminus, thereby producing characteristic pulsing patterns. The NAA is cleaved by an aminopeptidase, exposing the next amino acid for recognition. The temporal order of NAA recognition and the kinetics of binding enable peptide identification and are sensitive to features that modulate binding kinetics, such as post-translational modifications (PTMs).

FIG. 1B shows an example schematic of a pixel of an integrated device.

FIGS. 2A-2D show example results illustrating TET aminopeptidase performance improvement by extending the C terminal of the peptide with DDD motif. The bar charts in FIGS. 2A-2D show cut depth (FIG. 2A), cleavage activity (% of reads reaching the last visible RS) (FIG. 2B), time taken to cleave DQQ motif and R residue (FIG. 2C), and % of 4+RSs (FIG. 2D).

FIGS. 3A-3B show example results from AP30 expression and purification. FIG. 3A shows an example chromatogram showing AP30 separation and the eluted peak. FIG. 3B shows Talon affinity column purification fractions resolved on SDS PAGE gel to show AP30 enrichment (left image), and the native gel showing hTETII and AP30 protein profiles before and after conditioning (right image).

FIG. 4 shows three bar charts comparing the inherent cleavage rates and substrate specificities by hTETII and AP30 for 18 individual amino acids categorized as per the activity level (high activity: top chart; moderate activity: middle chart; very low activity: bottom chart).

FIGS. 5A-5C show example results from a protein sequencing assay with AP30 cutter (5 μM), PS610 (50 nM) recognizer and QP47 peptide (FAAAYPDDD) (SEQ ID NO: 46). FIG. 5A shows a representative trace showing the first RS and the last RS recognition by PS610 as the cleavage progressed. FIG. 5B shows plots showing the mean cleavage times for FA (left plot) and YP (right plot) RSs. FIG. 5C shows a plot showing the rapid sequential cleavage population and regular cleavage population.

FIGS. 6A-6C show example results from a protein sequencing assay with 1 μM hTETII cutter, PS610 (50 nM) recognizer and QP47 peptide (FAAAYPDDD) (SEQ ID NO: 46). FIG. 6A shows a representative trace showing the first and the last RSs by PS610 as the cleavage progressed. FIG. 6B shows plots showing the mean cleavage times for FA and YP RSs. FIG. 6C shows a plot showing the rapid sequential cleavage population and regular cleavage population.

FIGS. 7A-7B show example results from a protein sequencing assay with AP30 (1 μM)/PfuTET (40 μM), along with PS610 (50 nM), PS557 (250 nM) and PS621 (250 nM) recognizers and QP433 peptide (RLIFAYPDDD) (SEQ ID NO: 47). FIG. 7A shows a representative trace showing the five RSs identified by the recognizers as the cleavage progressed. FIG. 7B shows a Bin Ratio vs. Pulse duration plot with separated clusters for recognized RSs (top plot), and plots showing the cut depths and proportion of each RS recognized in the reads with (middle plot) and without (bottom plot) gap (no missed recognizable residue). FIG. 7A also shows SEQ ID NO: 54.

FIGS. 8A-8B show example results from a protein sequencing assay with AP30 (3 μM) or hTETII (2 μM)/PfuTET (40 μM) aminopeptidase combination, along with PS610 (50 nM) and PS557 (250 nM) recognizers and QP425 (LASSIAEANRFADIADYP) (SEQ ID NO: 48) on the same chip. FIG. 8A shows representative traces for reactions with AP30 and hTETII showing RSs identified by the recognizers as the cleavage progressed. FIG. 8B shows plots showing cut depths and proportions of each RS recognized in the reads without gap (no missed recognizable residue).

FIGS. 9A-9C show example results illustrating that AP30 improved cutting performance by increasing cleavage activity and reducing rapid sequential cleavage. The bar charts in FIGS. 9A-9C show cut depth (FIG. 9A), % of 4+ RSs reads (FIG. 9B), and rapid sequential cleavage of IA and FA RSs (FIG. 9C).

FIGS. 10A-10B show example results from AP37 expression and purification. FIG. 10A shows an example chromatogram showing AP37 enrichment and the eluted peak. FIG. 10B shows talon affinity column purification fractions resolved on SDS PAGE gel to show AP37 separation.

FIG. 11 shows three bar charts comparing the inherent cleavage rates and substrate specificities by PfuTET and AP37 for 18 individual amino acids categorized as per the activity level (high activity: top chart; moderate activity: middle chart; very low activity: bottom chart).

FIGS. 12A-12B show example results from a protein sequencing assay with AP37 (40 μM) or PfuTET (40 μM) aminopeptidase, along with PS610 (50 nM), PS961 (250 nM), PS961 (250 nM) recognizers and QP514 (DQQRLIFAYPDDD) (SEQ ID NO: 49) on separate chips. FIG. 12A shows representative traces for reaction with AP37 or PfuTET showing RSs identified by the recognizers as the cleavage progressed. FIG. 12B shows plots showing cut depths and proportions of each RS recognized in the reads without gap (no missed recognizable residue).

FIGS. 13A-13C show example results from a protein sequencing assay performed at two different AP37 concentrations (20 μM/40 μM), 4 μM hTETII, and PS610 (50 nM), PS557 (250 nM) and PS691 (100 nM) recognizers on QP433 (RLIFAYPDDD) (SEQ ID NO: 47). FIG. 13A shows the improvements in cut depth. FIG. 13B shows improvements in % of important reads. FIG. 13C shows that AP37 reduced rapid sequential cleavage (RSC) of LI, IF, and FA RSs.

FIGS. 14A-14B show example results illustrating the effect of different AP30/AP37 proportions with PS610 (50 nM), PS691 (100 nM), and PS961 (250 nM) recognizers, on time to cleave DQQ motif and R residue (FIG. 14A) and RS rapid sequential cleavage (FIG. 14B).

FIGS. 15A-15B show bar charts showing the distribution of the performance metrics (cut depth, % of 4+RSs reads, and % of 5 RSs reads) with the recognizers mix of PS610 (50 nM), PS961 (250 nM), and PS691 (100 nM), and AP30/AP37 at 4 μM/40 μM on QP514 (DQQRLIFAYPDDD) (SEQ ID NO: 49) (FIG. 15A) and at 10 μM/60 μM concentration on QP549 (DQQIASSRLAASFAAQQYPDDD) (SEQ ID NO: 50) (FIG. 15B).

FIG. 16 shows reads breakdown and abundance of ungapped reads, with single deletion allowed in reads of length of 4, obtained from one of the chip runs by the recognizers and aminopeptidases mix of PS961/PS691 at 250/100 nM and AP30/hTETIII at 10/60 μM, on QP549.

FIGS. 17A-17B show example results from yPIP expression and purification. FIG. 17A shows an example chromatogram showing yPIP separation and the eluted peak. FIG. 17B shows Talon affinity column purification fractions resolved on SDS PAGE gel to show yPIP enrichment.

FIGS. 18A-18D show representative traces showing the effect of adding yPIP (2 μm) in the AP30/AP37 (4/40 μM) aminopeptidase combination in a protein sequencing assay. SEQ ID NO: 55 is shown throughout the figure.

FIGS. 19A-19B show example results from protein sequencing assays with (FIG. 19A) AP30 (4 μM)/AP37 (40 μM), and (FIG. 19B) AP30 (4 μM)/AP37 (40 μM)/yPIP (2 μM) combinations on QP352 (RLAYPAFAAYPDDDF) (SEQ ID NO: 51) peptide along with PS610 (50 nM)/PS961 (125 nM)/PS1122 (250 nM) recognizers.

FIGS. 20A-20B show reads proportion breakdown for the QP352 (RLAYPAFAAYPDDDF) (SEQ ID NO: 51) protein sequencing assay with (FIG. 20A) AP30 (4 μM)/AP37 (40 μM), and (FIG. 20B) AP30 (4 μM)/AP37 (40 μM)/yPIP (2 μM) combinations along with PS610 (50 nM)/PS961 (125 nM)/PS1122 (250 nM) recognizers.

FIGS. 21A-21B show example results from protein sequencing assays with (FIG. 21A) AP30 (4 μM)/AP37 (40 μM)/yPIP (0.5 μM), and (FIG. 21B) AP30 (4 μM)/AP37 (40 μM)/yPIP (1 μM) combinations on QP352 (RLAYPAFAAYPDDDF) (SEQ ID NO: 51) peptide along with PS610 (50 nM)/PS961 (125 nM) recognizers.

FIGS. 22A-22B show reads proportion breakdown for the QP352 (RLAYPAFAAYPDDDF) (SEQ ID NO: 51) protein sequencing assay with (FIG. 22A) AP30 (4 μM)/AP37 (40 μM)/yPIP (0.5 μM), and (FIG. 22B) AP30 (4 μM)/AP37 (40 μM)/yPIP (1 μM) combinations along with PS610 (50 nM)/PS961 (125 nM) recognizers combinations along with PS610 (50 nM)/PS961 (125 nM) recognizers.

FIGS. 23A-23B show example results from protein sequencing assays with (FIG. 23A) AP30 (4 μM)/AP37 (40 μM))/yPIP-BC_B1 (2 μM), and (FIG. 23B) AP30 (4 μM)/AP37 (40 μM)/yPIP-470 (2 μM) combinations on QP352 (RLAYPAFAAYPDDDF) (SEQ ID NO: 51) peptide along with PS610 (50 nM)/PS961 (125 nM)/PS1122 (250 nM) recognizers.

FIGS. 24A-24B show reads proportion breakdown for the QP352 (RLAYPAFAAYPDDDF) (SEQ ID NO: 51) protein sequencing assay with (FIG. 24A) AP30 (4 μM)/AP37 (40 μM))/yPIP-BC_B1 (2 μM), and (FIG. 24B) AP30 (4 μM)/AP37 (40 μM)/yPIP-470 (2 μM) combinations along with PS610 (50 nM)/PS961 (125 nM)/PS1122 (250 nM) recognizers.

FIGS. 25A-25B show (FIG. 25A) yPIP (SEQ ID NO: 6) and AP70 (SEQ ID NO: 8) protein amino acid sequence alignment showing the C terminal 6×H tag and GGS-6×His-tag, respectively, and (FIG. 25B) expression constructs for yPIP and AP70 with molecular weights of the proteins.

FIGS. 26A-26B show example results from AP70 expression and purification. FIG. 26A shows an example chromatogram showing AP70 separation and the eluted peak. FIG. 26B shows talon affinity column purification fractions resolved on SDS PAGE gel to show AP70 enrichment.

FIGS. 27A-27D show example results from HPLC assay for QP734 (FPARAFAYPDDD) (SEQ ID NO: 52) peptide cleavage by AP70 at various concentrations after different conditioning treatments and unconditioned control.

FIGS. 28A-28D show example results from HPLC assay for different peptide cleavage by AP70 after different conditioning treatments and unconditioned control.

FIG. 29 shows example results comparing the cleavage activity and substrate specificities for AP70, AP30+AP37, and AP30+AP37+AP70 combinations on PXX, XPP, XPX peptides containing proline.

FIGS. 30A-30B show example results from protein sequencing assay with AP70 (1 μM, 4 μM), no AP control, AP30 (4 μM) on QP734 (FPARAFAYPDDD) (SEQ ID NO: 52) peptide along with PS610 (50 nM)/PS961 (125 nM)/PS1122 (250 nM) recognizers.

FIGS. 31A-31H show representative traces from protein sequencing assays showing the effect of adding AP70 (1 μm) in the AP30/AP37 (4/40 μM) aminopeptidase combination.

FIGS. 32A-32Q show example results from AP70 concentration titration to optimize the AP30+AP37+AP70 AP combination performance on chips for QP352 (RLAYPAFAAYPDDDF) (SEQ ID NO: 51) sequencing.

FIGS. 33A-33B show example results from protein sequencing assays with (FIG. 33A) AP30 (4 μM)/AP37 (40 μM)/AP70 (1 μM), and (FIG. 33B) AP30 (4 μM)/AP37 (40 μM) combinations on QP352 (RLAYPAFAAYPDDDF) (SEQ ID NO: 51) peptide along with PS610 (50 nM)/PS961 (125 nM)/PS1122 (250 nM) recognizers.

FIGS. 34A-34B show reads proportion breakdown for the QP352 (RLAYPAFAAYPDDDF) (SEQ ID NO: 51) protein sequencing assay with (FIG. 34A) AP30 (4 μM)/AP37 (40 μM)/AP70 (1 μM) and (FIG. 34B) AP30 (4 μM)/AP37 (40 μM) combinations along with PS610 (50 nM)/PS961 (125 nM)/PS1122 (250 nM) recognizers.

FIGS. 35A-35B show example results from protein sequencing assays with (FIG. 35A) AP30 (4 μM)/AP37 (40 μM)/AP70 (1 μM), and (FIG. 35B) AP30 (4 μM)/AP37 (40 μM) combinations on QP354 (LAAYPARLAYPDDDF) (SEQ ID NO: 53) peptide along with PS610 (50 nM)/PS961 (125 nM)/PS1122 (250 nM) recognizers.

FIGS. 36A-36B show reads proportion breakdown for the QP354 (LAAYPARLAYPDDDF) (SEQ ID NO: 53) protein sequencing assay with (FIG. 36A) AP30 (4 μM)/AP37 (40 μM)/AP70 (1 μM), and (FIG. 36B) AP30 (4 μM)/AP37 (40 μM) combinations along with PS610 (50 nM)/PS961 (125 nM)/PS1122 (250 nM) recognizers.

FIGS. 37A-37B show example results from protein sequencing assays with (FIG. 37A) AP30 (4 μM)/AP37 (40 μM)/AP70 BC-B1 batch (1 μM), and (FIG. 37B) AP30 (4 μM)/AP37 (40 μM)/yPIP BC-B1 batch (1 μM) combinations on QP352 (RLAYPAFAAYPDDDF) (SEQ ID NO: 51) peptide along with PS610 (50 nM)/PS961 (125 nM)/PS1122 (250 nM) recognizers.

FIGS. 38A-38B show reads proportion breakdown for the QP352 (RLAYPAFAAYPDDDF) (SEQ ID NO: 51) protein sequencing assays with (FIG. 38A) AP30 (4 μM)/AP37 (40 μM)/AP70 BC-B1 batch (1 μM), and (FIG. 38B) AP30 (4 μM)/AP37 (40 μM)/yPIP BC-B1 batch (1 μM) combinations along with PS610 (50 nM)/PS961 (125 nM)/PS1122 (250 nM) recognizers.

### DETAILED DESCRIPTION

Aspects of the disclosure relate to compositions and methods for polypeptide analysis based on single-molecule binding interactions between the polypeptide and one or more reagents described herein. In some embodiments, the disclosure provides cleaving reagents, such as aminopeptidases, having improved performance in peptide sequencing reactions. In some embodiments, cleaving reagents of the disclosure display improvements in cleavage activity toward amino acids of a polypeptide, allowing for more information to be obtained from peptide sequencing reactions.

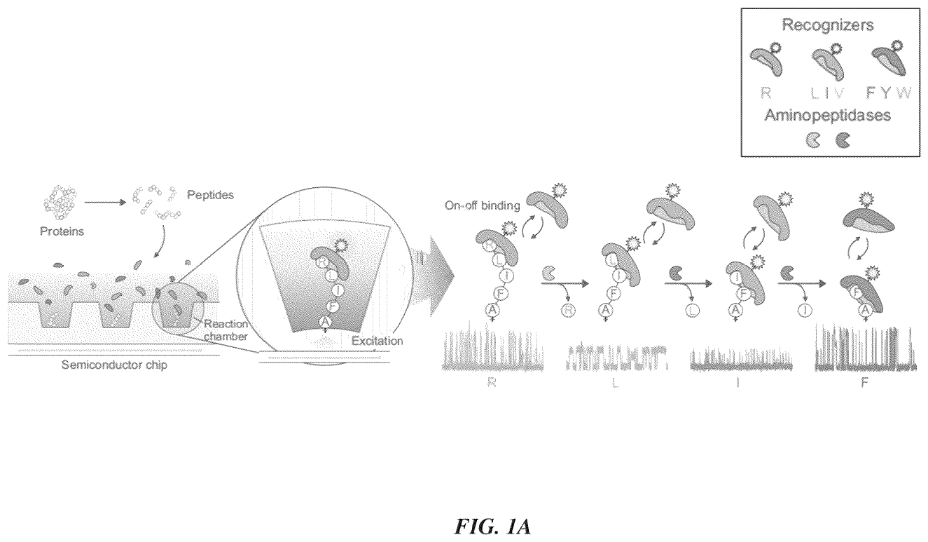

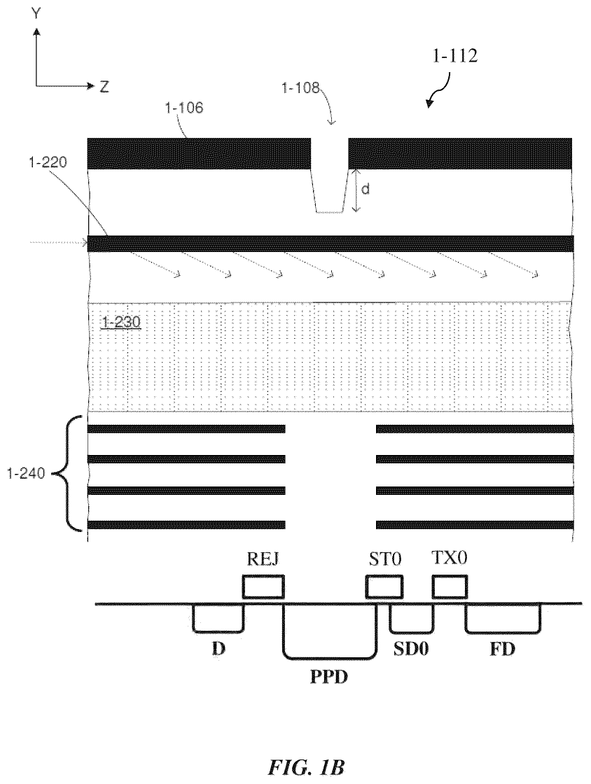

For example, FIG. 1A shows an example of a dynamic peptide sequencing reaction in which individual on-off binding events give rise to signal pulses of a signal output. As shown at left, a protein sample may be fragmented into peptides, which are immobilized in reaction chambers and exposed to a mixture of amino acid recognizers and cleaving reagents. As shown at right, amino acid recognizers reversibly bind to the peptide, producing a series of changes in signal output (e.g., signal pulses) as amino acids are progressively cleaved from the peptide terminus. The temporal order of recognition and the kinetics of binding and/or cleaving can be used to determine structural information for the peptide.

Compositions and methods for performing dynamic peptide sequencing and analyzing data obtained therefrom are described more fully in PCT International Publication No. WO2020/102741A1, filed Nov. 15, 2019, and PCT International Publication No. WO2021/236983A2, filed May 20, 2021, each of which is incorporated by reference in its entirety.

In some aspects, the disclosure provides cleaving reagents (e.g., aminopeptidases) with improved cleavage activity which can be advantageous in the context of a peptide sequencing reaction. As described herein and illustrated in FIG. 1A, peptide sequencing reactions may be carried out by exposing a polypeptide to a mixture of amino acid recognizers and aminopeptidases, and determining structural information for the polypeptide based on detectable recognition events preceding a cleavage event. In some embodiments, aminopeptidases of the disclosure allow for more information to be obtained from sequencing reactions. For example, in some embodiments, the aminopeptidases display cleavage activity at improved rates for detecting recognition events in between cleavage events (e.g., as shown by a decrease in rapid sequential cleavage). In some embodiments, the sequential cleavage of amino acids by the aminopeptidases progresses further into peptide substrates (e.g., as shown by an increase in cut depth). In some embodiments, the aminopeptidases cleave certain amino acids more efficiently relative to a homologous enzyme.

### Aminopeptidases

In some aspects, the disclosure provides a cleaving reagent comprising an aminopeptidase having an amino acid sequence selected from Table 1. It should be appreciated that the example sequences in Table 1 and other examples described herein are meant to be non-limiting, and aminopeptidases in accordance with the disclosure can include any homologs, variants, or fragments thereof minimally containing domains or subdomains responsible for amino acid cleavage.

In some embodiments, a cleaving reagent comprises an aminopeptidase having an amino acid sequence that is at least 80% identical to an amino acid sequence selected from Table 1. In some embodiments, an aminopeptidase has at least 40%, at least 50%, at least 60%, at least 70%, at least 80%, at least 85%, at least 90%, at least 92%, at least 94%, at least 95%, at least 96%, at least 98%, or higher, amino acid sequence identity to an amino acid sequence selected from Table 1. In some embodiments, an aminopeptidase has 25-50%, 50-60%, 60-70%, 70-80%, 80-90%, 90-95%, 92-99%, 94-99%, 95-99%, 40-100%, 50-100%, 60-100%, 70-100%, 80-100%, 90-100%, 92-100%, 94-100%, 95-100%, 96-100%, or 100% amino acid sequence identity to an amino acid sequence selected from Table 1.

In some embodiments, a cleaving reagent comprises a synthetic or recombinant aminopeptidase. In some embodiments, a cleaving reagent comprises a monomeric aminopeptidase. In some embodiments, a cleaving reagent comprises a multimeric aminopeptidase (e.g., a multimeric complex of monomeric subunits, which may be the same or different).

In some embodiments, a cleaving reagent comprises an aminopeptidase obtained or derived from a particular source (e.g., organism). As described herein, in some embodiments, an aminopeptidase identified as being from a particular organism does not impart a requirement that the aminopeptidase have an amino acid sequence that is 100% identical to a naturally-occurring aminopeptidase from the organism, although it may in some embodiments.

For example, in some embodiments, a cleaving reagent comprises an aminopeptidase from Pyrococcus horikoshii (e.g., Pyrococcus horikoshii TET Aminopeptidase II, Pyrococcus horikoshii TET Aminopeptidase III). In some embodiments, an aminopeptidase from Pyrococcus horikoshii is at least 80%, at least 85%, at least 90%, at least 95%, 80-100%, 85-95%, 90-99%, 95-99%, or 100% identical to a naturally-occurring aminopeptidase from Pyrococcus horikoshii (e.g., Pyrococcus horikoshii TET Aminopeptidase II, Pyrococcus horikoshii TET Aminopeptidase III).

In some embodiments, a cleaving reagent comprises an aminopeptidase from Yersinia pestis (e.g., Yersinia pestis Xaa-Prolyl Aminopeptidase). In some embodiments, an aminopeptidase from Yersinia pestis is at least 80%, at least 85%, at least 90%, at least 95%, 80-100%, 85-95%, 90-99%, 95-99%, or 100% identical to a naturally-occurring aminopeptidase from Yersinia pestis (e.g., Yersinia pestis Xaa-Prolyl Aminopeptidase).

In some embodiments, a cleaving reagent comprises an aminopeptidase from Pyrococcus furiosus (e.g., Pyrococcus furiosus Aminopeptidase I). In some embodiments, an aminopeptidase from Pyrococcus furiosus is at least 80%, at least 85%, at least 90%, at least 95%, 80-100%, 85-95%, 90-99%, 95-99%, or 100% identical to a naturally-occurring aminopeptidase from Pyrococcus furiosus (e.g., Pyrococcus furiosus Aminopeptidase I).

### Tag Sequences

In some embodiments, a cleaving reagent comprises an aminopeptidase described herein and a tag sequence. As used herein, in some embodiments, a tag sequence refers to a segment of amino acids attached to an aminopeptidase. In some embodiments, a tag sequence is attached to a terminal end (e.g., terminus) of an aminopeptidase. In some embodiments, a tag sequence is attached to the C-terminus of an aminopeptidase (e.g., the C-terminal amino acid of the aminopeptidase). In some embodiments, a tag sequence is attached to the N-terminus of an aminopeptidase (e.g., the N-terminal amino acid of the aminopeptidase). In some embodiments, a tag sequence is attached to an internal position of an aminopeptidase (e.g., an amino acid between the N- and C-terminal amino acids of the aminopeptidase).

In some embodiments, a tag sequence comprises at least two amino acids. For example, in some embodiments, a tag sequence comprises at least 2, at least 3, at least 4, at least 5, at least 6, at least 8, at least 10, at least 15, at least 25, at least 30, at least 40, at least 50, at least 60, at least 80, at least 100, or more, amino acids. In some embodiments, a tag sequence comprises between about 2 and about 200 amino acids (e.g., 2-150 amino acids, 2-100 amino acids, 50-200 amino acids, 50-150 amino acids, 50-100 amino acids, 4-80 amino acids, 5-50 amino acids, 5-30 amino acids, 5-20 amino acids, 10-100 amino acids, 20-80 amino acids, 30-70 amino acids).

In some embodiments, a tag sequence comprises one or more functional components. In some embodiments, a functional component provides improvements in cleavage activity of the aminopeptidase to which it is attached. For example, in some embodiments, a cleaving reagent comprises a tag sequence that improves one or more properties related to aminopeptidase cleavage activity, such as improvements in one or more of cut depth, rapid sequential cleavage, and cutting efficiency. In some embodiments, a functional component of a tag sequence can provide one or more functions unrelated to aminopeptidase cleavage activity. For example, in some embodiments, a tag sequence comprises one or more of an affinity tag (e.g., a polyhistidine tag), a modification tag (e.g., a biotinylation tag), a solubility tag (e.g., small ubiquitin-like modifier (SUMO) tag), and a linker.

In some embodiments, a tag sequence comprises a polyhistidine-tag. In some embodiments, a polyhistidine-tag comprises a segment of two or more histidine amino acids. In some embodiments, a polyhistidine-tag comprises a segment of between about 2 and about 15 (e.g., 4, 6, 8, 10, 12, 14, 15) histidine amino acids. In some embodiments, a polyhistidine-tag comprises a hexahistidine-tag (e.g., 6×His-tag). In some embodiments, a polyhistidine-tag comprises a decahistidine-tag (e.g., 10×His-tag). In some embodiments, a tag sequence comprises two or more (e.g., two, three, four) polyhistidine-tags.

In some embodiments, a tag sequence comprises a biotinylation tag. In some embodiments, a biotinylation tag comprises at least one biotin ligase recognition sequence. In some embodiments, a biotinylation tag comprises two biotin ligase recognition sequences oriented in tandem. In some embodiments, a biotin ligase recognition sequence refers to an amino acid sequence that can be recognized by a biotin ligase, which catalyzes a covalent linkage between the sequence and a biotin molecule. In some embodiments, a biotin ligase recognition sequence comprises an amino acid sequence of SEQ ID NO: 36. In some embodiments, a tag sequence comprises two or more (e.g., two, three, four) biotin ligase recognition sequences.

In some embodiments, a tag sequence comprises at least one polyhistidine-tag and at least one biotin ligase recognition sequence. In some embodiments, a tag sequence comprises at least one polyhistidine-tag and at least two biotin ligase recognition sequences. In some embodiments, a tag sequence comprises at least two polyhistidine-tags and at least one biotin ligase recognition sequence.

In some embodiments, a tag sequence comprises a solubility tag. In some embodiments, a tag sequence comprises a tag peptide or tag protein. Examples of tag peptides include, without limitation, calmodulin-binding peptide (CBP) tag, FLAG epitope, human influenza hemagglutinin (HA) tag, Myc epitope, streptavidin-binding peptide, Strep tag, Strep-II tag, intrinsically disordered tag, Fasciola hepatica 8-kDa antigen (Fh8), maltose-binding protein (MBP), N-utilization substance (NusA), thioredoxin (Trx), small ubiquitin-like modifier (SUMO), glutathione-S-transferase (GST), solubility-enhancer peptide sequences (SET), IgG domain B1 of Protein G (GB1), IgG repeat domain ZZ of Protein A (ZZ), mutated dehalogenase (HaloTag), Solubility eNhancing Ubiquitous Tag (SNUT), seventeen kilodalton protein (Skp), bacteriophage V5 epitope, phage T7 protein kinase (T7PK), E. coli secreted protein A (EspA), monomeric bacteriophage T7 0.3 protein (Orc protein; Mocr), E. coli trypsin inhibitor (Ecotin), calcium-binding protein (CaBP), stress-responsive arsenate reductase (ArsC), N-terminal fragment of translation initiation factor IF2 (IF2-domain I), stress-responsive proteins (e.g., RpoA, SlyD, Tsf, RpoS, PotD, Crr), and E. coli acidic proteins (e.g., msyB, yjgD, rpoD). Additional examples of tags are known in the art and may be used in accordance with the disclosure. See, e.g., Costa, S., et al. “Fusion tags for protein solubility, purification and immunogenicity in Escherichia coli: the novel Fh8 system.” Front Microbiol. 2014 Feb. 19; 5:63; and Kimple, M. E., et al. “Overview of Affinity Tags for Protein Purification.” Curr Protoc Protein Sci. 2013, 73: Unit 9-9, the relevant contents of which are incorporated by reference herein.

In some embodiments, a tag sequence comprises a linker. For example, in some embodiments, a tag sequence comprises a polyhistidine-tag and a linker between the polyhistidine-tag and an amino acid of an aminopeptidase. In some embodiments, a tag sequence comprises a biotin ligase recognition sequence and a linker between the biotin ligase recognition sequence and an amino acid of an aminopeptidase. In some embodiments, a tag sequence comprises a biotin ligase recognition sequence, a polyhistidine-tag, and a linker between the biotin ligase recognition sequence and the polyhistidine-tag. In some embodiments, a tag sequence comprises two biotin ligase recognition sequences and a linker between the two biotin ligase recognition sequences.

In some embodiments, a linker of a tag sequence comprises one or more amino acids. In some embodiments, a linker comprises at least 2, at least 3, at least 4, at least 5, at least 6, at least 8, at least 10, at least 12, at least 15, at least 30, at least 25, at least 30, or more, amino acids. In some embodiments, a linker comprises between about 1 and about 50 amino acids (e.g., 1-50 amino acids, 1-30 amino acids, 2-25 amino acids, 3-30 amino acids, 6-25 amino acids, 2-15 amino acids, 1-12 amino acids).

In some embodiments, a linker of a tag sequence comprises at least one glycine amino acid. In some embodiments, a linker comprises at least one glycine-serine (GS) motif. In some embodiments, a linker comprises an amino acid sequence of the following formula: (GₘS)ₙ, where: G is glycine; S is serine; m is an integer from 1 to 5, inclusive; and n is an integer from 1 to 6, inclusive. In some embodiments, m is an integer from 1 to 3, inclusive. In some embodiments, m is 2 or 3. In some embodiments, m is 2. In some embodiments, m is 3. In some embodiments, n is an integer from 1 to 4, inclusive. In some embodiments, n is an integer from 1 to 3, inclusive. In some embodiments, n is 1 or 3. In some embodiments, n is 1. In some embodiments, n is 3.

In some embodiments, a cleaving reagent comprises a tag sequence having an amino acid sequence selected from Table 2. In some embodiments, a cleaving reagent comprises a tag sequence having an amino acid sequence that is at least 40% identical to an amino acid sequence selected from Table 2. In some embodiments, a tag sequence has at least 45%, at least 50%, at least 60%, at least 70%, at least 80%, at least 85%, at least 90%, at least 92%, at least 94%, at least 95%, at least 96%, at least 98%, or higher, amino acid sequence identity to an amino acid sequence selected from Table 2. In some embodiments, a tag sequence has 25-50%, 50-60%, 60-70%, 70-80%, 80-90%, 90-95%, 92-99%, 94-99%, 95-99%, 40-100%, 50-100%, 60-100%, 70-100%, 80-100%, 90-100%, 92-100%, 94-100%, 95-100%, 96-100%, or 100% amino acid sequence identity to an amino acid sequence selected from Table 2.

Accordingly, in some embodiments, a cleaving reagent comprises an aminopeptidase and a tag sequence, where the aminopeptidase has an amino acid sequence selected from Table 1, and where the tag sequence has an amino acid sequence selected from Table 2. In some embodiments, a cleaving reagent comprises an aminopeptidase and a tag sequence, where the aminopeptidase has an amino acid sequence that is at least 80% identical to an amino acid sequence selected from Table 1, and where the tag sequence has an amino acid sequence that is at least 40% identical to an amino acid sequence selected from Table 2.

In some aspects, the disclosure provides a single polypeptide comprising an aminopeptidase described herein attached to a tag sequence described herein. For example, in some embodiments, a cleaving reagent comprises a fusion polypeptide of an aminopeptidase fused to a tag sequence. In some embodiments, a fusion polypeptide comprises an aminopeptidase and a tag sequence fused to a terminal end of the aminopeptidase. In some embodiments, a fusion polypeptide comprises an aminopeptidase and a tag sequence fused to the C-terminal end of the aminopeptidase. In some embodiments, a fusion polypeptide comprises an aminopeptidase and a tag sequence fused to the N-terminal end of the aminopeptidase. In some aspects, the disclosure provides a nucleic acid encoding a cleaving reagent described herein. In some embodiments, the nucleic acid is an expression construct encoding a fusion polypeptide of an aminopeptidase fused to a tag sequence.

### Compositions and Reaction Mixtures

In some aspects, the disclosure provides compositions comprising at least one cleaving reagent described herein. In some embodiments, a composition comprises two or more cleaving reagents, where at least one cleaving reagent comprises an aminopeptidase and a tag sequence described herein. In some embodiments, a composition comprises two or more cleaving reagents described herein.

In some aspects, the disclosure provides a composition comprising: a first cleaving reagent comprising a first aminopeptidase from Pyrococcus horikoshii and a tag sequence (e.g., first tag sequence) described herein; and a second cleaving reagent comprising a second aminopeptidase from Pyrococcus horikoshii, where the first and second aminopeptidases have different amino acid sequences. In some embodiments, the amino acid sequences of the first and second aminopeptidases share less than 80% sequence identity (e.g., less than 70%, less than 60%, less than 50%, 10-80%, 20-60%, 30-50%, or 40-50% sequence identity). In some embodiments, the second cleaving reagent comprises a tag sequence (e.g., second tag sequence) described herein. In some embodiments, the composition comprises a third cleaving reagent. In some embodiments, the third cleaving reagent comprises an aminopeptidase from Yersinia pestis.

In some embodiments, the first and second cleaving reagents are present in the composition at a first and second concentration, respectively, where the first concentration is at least two-fold higher than the second concentration. In some embodiments, the molar ratio of the first cleaving reagent to the second cleaving reagent in the composition is between about 2:1 and about 20:1 (e.g., between about 2:1 and about 15:1, between about 2:1 and about 10:1, between about 4:1 and about 15:1, or between about 5:1 and about 10:1). In some embodiments, the molar ratio of the first cleaving reagent to the second cleaving reagent in the composition is about 2:1, about 4:1, about 6:1, about 8:1, or about 10:1.

In some embodiments, the first cleaving reagent is present at a concentration of between about 10 μM and about 100 μM (e.g., 20-80 μM, 20-60 μM, 10-30 μM, 30-50 μM, 50-70 μM, 70-90 μM). In some embodiments, the first cleaving reagent is present at a concentration of about 20 μM, about 40 μM, about 60 μM, or about 80 μM. In some embodiments, the first cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the N-terminal amino acid comprises a charged side chain (e.g., arginine, lysine, glutamine, aspartate, glutamate).

In some embodiments, the second cleaving reagent is present at a concentration of between about 0.1 μM and about 25 μM (e.g., 0.1-10 μM, 0.5-20 μM, 1-10 μM, 2-20 μM, 2-15 μM, 1-8 μM). In some embodiments, the second cleaving reagent is present at a concentration of about 2 μM, about 4 μM, about 6 μM, or about 8 μM. In some embodiments, the second cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the N-terminal amino acid comprises a hydrophobic side chain (e.g., alanine, valine, isoleucine, leucine, methionine, phenylalanine, tyrosine, tryptophan).

In some embodiments, the first aminopeptidase comprises one or more substitutions relative to Pyrococcus horikoshii TET Aminopeptidase III. In some embodiments, the first aminopeptidase has an amino acid sequence that is at least 80% (e.g., at least 85%, at least 90%, at least 95%, 80-100%, 85-95%, 90-99%, 95-99%, or 100%) identical to SEQ ID NO: 3. In some embodiments, the first cleaving reagent is a polypeptide having an amino acid sequence that is at least 80% (e.g., at least 85%, at least 90%, at least 95%, 80-100%, 85-95%, 90-99%, 95-99%, or 100%) identical to SEQ ID NO: 4.

In some embodiments, the second aminopeptidase comprises one or more substitutions relative to Pyrococcus horikoshii TET Aminopeptidase II. In some embodiments, the second aminopeptidase has an amino acid sequence that is at least 80% (e.g., at least 85%, at least 90%, at least 95%, 80-100%, 85-95%, 90-99%, 95-99%, or 100%) identical to SEQ ID NO: 1. In some embodiments, the second cleaving reagent is a polypeptide having an amino acid sequence that is at least 80% (e.g., at least 85%, at least 90%, at least 95%, 80-100%, 85-95%, 90-99%, 95-99%, or 100%) identical to SEQ ID NO: 2.

In some aspects, the disclosure provides a composition comprising a first cleaving reagent comprising a first aminopeptidase having an amino acid sequence that is at least 80% identical to SEQ ID NO: 3; a second cleaving reagent comprising a second aminopeptidase having an amino acid sequence that is at least 80% identical to SEQ ID NO: 1; and a third cleaving reagent comprising a third aminopeptidase having an amino acid sequence that is at least 80% identical to SEQ ID NO: 5 or 7.

In some embodiments, at least one of the first, second, and third cleaving reagents comprises a tag sequence described herein. For example, in some embodiments, the first cleaving reagent comprises a first tag sequence; the second cleaving reagent comprises a second tag sequence; and the third cleaving reagent comprises a third tag sequence. In some embodiments, the first tag sequence is attached to a terminal end of the first aminopeptidase; the second tag sequence is attached to a terminal end of the second aminopeptidase; and the third tag sequence is attached to a terminal end of the third aminopeptidase. In some embodiments, each tag sequence is attached to the C-terminus of its respective aminopeptidase.

In some embodiments, the first, second, and third cleaving reagents are present in the composition at a first, second, and third concentration, respectively, where the first concentration is at least two-fold higher than the second concentration. In some embodiments, the first concentration is at least five-fold higher than the third concentration. In some embodiments, the second concentration is at least two-fold higher than the third concentration.

In some embodiments, the molar ratio of the first cleaving reagent to the second cleaving reagent in the composition is between about 2:1 and about 20:1 (e.g., between about 2:1 and about 15:1, between about 2:1 and about 10:1, between about 4:1 and about 15:1, or between about 5:1 and about 10:1). In some embodiments, the molar ratio of the first cleaving reagent to the third cleaving reagent in the composition is between about 5:1 and about 200:1 (e.g., between about 5:1 and about 150:1, between about 5:1 and about 100:1, between about 10:1 and about 80:1, or between about 10:1 and about 50:1).

In some embodiments, the first cleaving reagent is present at a concentration of between about 10 μM and about 100 μM (e.g., 20-80 μM, 20-60 μM, 10-30 μM, 30-50 μM, 50-70 μM, 70-90 μM). In some embodiments, the first cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the N-terminal amino acid comprises a charged side chain (e.g., arginine, lysine, glutamine, aspartate, glutamate).

In some embodiments, the second cleaving reagent is present at a concentration of between about 0.1 μM and about 25 μM (e.g., 0.1-10 μM, 0.5-20 μM, 1-10 μM, 2-20 μM, 2-15 μM, 1-8 μM). In some embodiments, the second cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the N-terminal amino acid comprises a hydrophobic side chain (e.g., alanine, valine, isoleucine, leucine, methionine, phenylalanine, tyrosine, tryptophan).

In some embodiments, the third cleaving reagent is present at a concentration of between about 0.01 μM and about 25 μM (e.g., 0.1-10 μM, 0.5-20 μM, 1-10 μM, 2-20 μM, 2-15 μM, 1-8 μM). In some embodiments, the third cleaving reagent is present in an amount sufficient to cleave an N-terminal amino acid from a polypeptide with an average cleavage time of between about 2 and about 60 minutes (e.g., 5-50 minutes, 5-30 minutes, 5-20 minutes, 5-15 minutes, 5-10 minutes, 2-30 minutes, 10-30 minutes, 30-60 minutes), where the polypeptide comprises an XP dipeptide motif, where: X is the N-terminal amino acid, and P is a proline amino acid.

In some embodiments, the amino acid sequence of the first aminopeptidase is at least 85%, at least 90%, at least 95%, 80-100%, 85-95%, 90-99%, 95-99%, or 100% identical to SEQ ID NO: 3. In some embodiments, the first cleaving reagent is a polypeptide having an amino acid sequence that is at least 80% (e.g., at least 85%, at least 90%, at least 95%, 80-100%, 85-95%, 90-99%, 95-99%, or 100%) identical to SEQ ID NO: 4.

In some embodiments, the amino acid sequence of the second aminopeptidase is at least 85%, at least 90%, at least 95%, 80-100%, 85-95%, 90-99%, 95-99%, or 100% identical to SEQ ID NO: 1. In some embodiments, the second cleaving reagent is a polypeptide having an amino acid sequence that is at least 80% (e.g., at least 85%, at least 90%, at least 95%, 80-100%, 85-95%, 90-99%, 95-99%, or 100%) identical to SEQ ID NO: 2.

In some embodiments, the amino acid sequence of the third aminopeptidase is at least 85%, at least 90%, at least 95%, 80-100%, 85-95%, 90-99%, 95-99%, or 100% identical to SEQ ID NO: 5 or 7. In some embodiments, the third cleaving reagent is a polypeptide having an amino acid sequence that is at least 80% (e.g., at least 85%, at least 90%, at least 95%, 80-100%, 85-95%, 90-99%, 95-99%, or 100%) identical to SEQ ID NO: 8.

In some aspects, the disclosure provides a reaction mixture for polypeptide analysis, the reaction mixture comprising: a composition described herein; and one or more amino acid recognizers (e.g., one or more amino acid binding proteins not having peptide cleavage activity).

In some embodiments, an amino acid recognizer comprises an amino acid binding protein, such as a ClpS protein (e.g., Planctomycetia bacterium ClpS protein), a UBR protein (e.g., Kluyveromyces marxianus UBR protein), an Ntaql protein (e.g., Scleropages formosus Ntaql protein), or a variant or homolog thereof. In some embodiments, an amino acid recognizer comprises a label (e.g., a detectable label, such as a luminescent label). Examples of amino acid recognizers (e.g., recognition molecules) are described in detail in PCT International Publication No. WO2020/102741A1, filed Nov. 15, 2019, PCT International Publication No. WO2021/236983A2, filed May 20, 2021, and co-pending U.S. Ser. No. 63/395,328, filed Aug. 4, 2022, the relevant content of each of which is incorporated by reference in its entirety.

In some aspects, the disclosure provides a reaction mixture for polypeptide analysis, the reaction mixture comprising: a first aminopeptidase having an amino acid sequence that is at least 92% identical to SEQ ID NO: 3 and comprising a first tag sequence; and one or more amino acid binding proteins not having peptide cleavage activity. In some embodiments, the amino acid sequence of the first aminopeptidase is at least 94%, at least 96%, at least 98%, or 100% identical to SEQ ID NO: 3. In some embodiments, the reaction mixture further comprises one or more cleaving reagents described herein.

As described herein, compositions and reaction mixtures of the disclosure can be used to determine at least one chemical characteristic of a polypeptide based on a characteristic pattern. In some embodiments, polypeptide sequencing reaction conditions can be configured to achieve a time interval that allows for sufficient association events which provide a desired confidence level with a characteristic pattern. This can be achieved, for example, by configuring the reaction conditions based on various properties, including: reagent concentration, molar ratio of one reagent to another (e.g., ratio of amino acid recognizer to cleaving reagent, ratio of one recognizer to another, ratio of one cleaving reagent to another), number of different reagent types (e.g., the number of different types of recognizers and/or cleaving reagents, the number of recognizer types relative to the number of cleaving reagent types), cleavage activity (e.g., aminopeptidase activity), binding properties (e.g., kinetic and/or thermodynamic binding parameters for recognition molecule binding), reagent modification (e.g., polyol and other recognizer modifications which can alter interaction dynamics), reaction mixture components (e.g., one or more components, such as pH, buffering agent, salt, divalent cation, surfactant, and other reaction mixture components described herein), temperature of the reaction, and various other parameters apparent to those skilled in the art, and combinations thereof. The reaction conditions can be configured based on one or more aspects described herein, including, for example, signal pulse information (e.g., pulse duration, interpulse duration, change in magnitude), labeling strategies (e.g., number and/or type of fluorophore, linkers with or without shielding element), surface modification (e.g., modification of sample well surface, including polypeptide immobilization), sample preparation (e.g., polypeptide fragment size, polypeptide modification for immobilization), and other aspects described herein.

In some embodiments, a polypeptide sequencing reaction in accordance with the disclosure is performed under conditions in which recognition and cleavage of amino acids can occur simultaneously in a single reaction mixture. For example, in some embodiments, a polypeptide sequencing reaction is performed in a reaction mixture having a pH at which association events and cleavage events can occur. Accordingly, in some embodiments, a reaction mixture has a pH of between about 6.5 and about 9.0. In some embodiments, a reaction mixture has a pH of between about 7.0 and about 8.5 (e.g., between about 7.0 and about 8.0, between about 7.5 and about 8.5, between about 7.5 and about 8.0, or between about 8.0 and about 8.5).

In some embodiments, a polypeptide sequencing reaction is performed in a reaction mixture comprising one or more buffering agents. In some embodiments, a reaction mixture comprises a buffering agent in a concentration of at least 10 mM (e.g., at least 20 mM and up to 250 mM, at least 50 mM, 10-250 mM, 10-100 mM, 20-100 mM, 50-100 mM, or 100-200 mM).

In some embodiments, a reaction mixture comprises a buffering agent in a concentration of between about 10 mM and about 50 mM (e.g., between about 10 mM and about 25 mM, between about 25 mM and about 50 mM, or between about 20 mM and about 40 mM). Examples of buffering agents include, without limitation, HEPES (4-(2-hydroxyethyl)-1-piperazineethanesulfonic acid), Tris (tris(hydroxymethyl)aminomethane), and MOPS (3-(N-morpholino)propanesulfonic acid).

In some embodiments, a polypeptide sequencing reaction is performed in a reaction mixture comprising salt in a concentration of at least 10 mM. In some embodiments, a reaction mixture comprises salt in a concentration of at least 10 mM (e.g., at least 20 mM, at least 50 mM, at least 100 mM, or more). In some embodiments, a reaction mixture comprises salt in a concentration of between about 10 mM and about 250 mM (e.g., between about 20 mM and about 200 mM, between about 50 mM and about 150 mM, between about 10 mM and about 50 mM, or between about 10 mM and about 100 mM). Examples of salts include, without limitation, sodium salts, potassium salts, and acetates, such as sodium chloride (NaCl), sodium acetate (NaOAc), and potassium acetate (KOAc).

Additional examples of components for use in a reaction mixture include divalent cations (e.g., Mg²⁺, Co²⁺) and surfactants (e.g., polysorbate 20). In some embodiments, a reaction mixture comprises a divalent cation in a concentration of between about 0.1 mM and about 50 mM (e.g., between about 10 mM and about 50 mM, between about 0.1 mM and about 10 mM, or between about 1 mM and about 20 mM). In some embodiments, a reaction mixture comprises a surfactant in a concentration of at least 0.01% (e.g., between about 0.01% and about 0.10%). In some embodiments, a reaction mixture comprises one or more components useful in single-molecule analysis, such as an oxygen-scavenging system (e.g., a PCA/PCD system or a Pyranose oxidase/Catalase/glucose system) and/or one or more triplet state quenchers (e.g., trolox, COT, and NBA).

In some embodiments, a polypeptide sequencing reaction is performed at a temperature at which association events and cleavage events can occur. In some embodiments, a polypeptide sequencing reaction is performed at a temperature of at least 10° C. In some embodiments, a polypeptide sequencing reaction is performed at a temperature of between about 10° C. and about 50° C. (e.g., 15-45° C., 20-40° C., at or around 25° C., at or around 30° C., at or around 35° C., at or around 37° C.). In some embodiments, a polypeptide sequencing reaction is performed at or around room temperature.

As detailed above, a real-time sequencing process as illustrated by FIG. 1A can generally involve cycles of amino acid recognition and terminal amino acid cleavage. In some embodiments, the relative occurrence of recognition and cleavage can be controlled by a concentration differential between one or more amino acid recognizers and at least one cleaving reagent. In some embodiments, the concentration differential can be optimized such that the number of signal pulses detected during recognition of an individual amino acid provides a desired confidence interval for identification. For example, if an initial sequencing reaction provides signal data with too few signal pulses between cleavage events to permit determination of characteristic patterns with a desired confidence interval, the sequencing reaction can be repeated using a decreased concentration of non-specific exopeptidase relative to recognition molecule.

In some embodiments, polypeptide analysis in accordance with the disclosure may be carried out by contacting a polypeptide with a reaction mixture comprising one or more amino acid recognizers and one or more cleaving reagents (e.g., peptidases). In some embodiments, a reaction mixture comprises an amino acid recognizer at a concentration of between about 10 nM and about 10 μM. In some embodiments, a reaction mixture comprises a cleaving reagent at a concentration of between about 500 nM and about 500 μM.

In some embodiments, a reaction mixture comprises an amino acid recognizer at a concentration of between about 100 nM and about 10 μM, between about 250 nM and about 10 μM, between about 100 nM and about 1 μM, between about 250 nM and about 1 μM, between about 250 nM and about 750 nM, or between about 500 nM and about 1 μM. In some embodiments, a reaction mixture comprises an amino acid recognizer at a concentration of about 100 nM, about 250 nM, about 500 nM, about 750 nM, or about 1 μM. In some embodiments, a reaction mixture comprises a cleaving reagent at a concentration of between about 500 nM and about 250 μM, between about 500 nM and about 100 μM, between about 1 μM and about 100 μM, between about 500 nM and about 50 μM, between about 1 μM and about 100 μM, between about 10 μM and about 200 μM, or between about 10 μM and about 100 μM. In some embodiments, a reaction mixture comprises a cleaving reagent at a concentration of about 1 μM, about 5 μM, about 10 μM, about 30 μM, about 50 μM, about 70 μM, or about 100 μM.

In some embodiments, a reaction mixture comprises an amino acid recognizer at a concentration of between about 10 nM and about 10 μM, and a cleaving reagent at a concentration of between about 500 nM and about 500 μM. In some embodiments, a reaction mixture comprises an amino acid recognizer at a concentration of between about 100 nM and about 1 μM, and a cleaving reagent at a concentration of between about 1 μM and about 100 μM.

In some embodiments, a reaction mixture comprises an amino acid recognizer at a concentration of between about 250 nM and about 1 μM, and a cleaving reagent at a concentration of between about 10 μM and about 100 μM. In some embodiments, a reaction mixture comprises an amino acid recognizer at a concentration of about 500 nM, and a cleaving reagent at a concentration of between about 25 μM and about 75 μM. In some embodiments, the concentration of an amino acid recognizer and/or the concentration of a cleaving reagent in a reaction mixture is as described elsewhere herein.

In some embodiments, a reaction mixture comprises an amino acid recognizer and a cleaving reagent in a molar ratio of about 500:1, about 400:1, about 300:1, about 200:1, about 100:1, about 75:1, about 50:1, about 25:1, about 10:1, about 5:1, about 2:1, or about 1:1. In some embodiments, a reaction mixture comprises an amino acid recognizer and a cleaving reagent in a molar ratio of between about 10:1 and about 200:1. In some embodiments, a reaction mixture comprises an amino acid recognizer and a cleaving reagent in a molar ratio of between about 50:1 and about 150:1. In some embodiments, the molar ratio of an amino acid recognizer to a cleaving reagent in a reaction mixture is between about 1:1,000 and about 1:1 or between about 1:1 and about 100:1 (e.g., 1:1,000, about 1:500, about 1:200, about 1:100, about 1:10, about 1:5, about 1:2, about 1:1, about 5:1, about 10:1, about 50:1, about 100:1). In some embodiments, the molar ratio of an amino acid recognizer to a cleaving reagent in a reaction mixture is between about 1:100 and about 1:1 or between about 1:1 and about 10:1. In some embodiments, the molar ratio of an amino acid recognizer to a cleaving reagent in a reaction mixture is as described elsewhere herein.

In some embodiments, a reaction mixture comprises one or more amino acid recognizers and one or more cleaving reagents described herein. In some embodiments, a reaction mixture comprises at least three amino acid recognizers and at least one cleaving reagent. In some embodiments, the reaction mixture comprises two or more cleaving reagents. In some embodiments, the reaction mixture comprises at least one and up to ten cleaving reagents (e.g., 1-3 cleaving reagents, 2-10 cleaving reagents, 1-5 cleaving reagents, 3-10 cleaving reagents). In some embodiments, the reaction mixture comprises at least three and up to thirty amino acid recognizers (e.g., between 3 and 25, between 3 and 20, between 3 and 10, between 3 and 5, between 5 and 30, between 5 and 20, between 5 and 10, or between 10 and 20, amino acid recognizers).

In some embodiments, a reaction mixture comprises more than one amino acid recognizer and/or more than one cleaving reagent. In some embodiments, a reaction mixture described as comprising more than one amino acid recognizer or cleaving reagent refers to the mixture as having more than one type of amino acid recognizer or cleaving reagent. For example, in some embodiments, a reaction mixture comprises two or more cleaving reagents, where the two or more cleaving reagents refer to two or more types of aminopeptidases. In some embodiments, one type of aminopeptidase has an amino acid sequence that is different from another type of aminopeptidase in the reaction mixture. In some embodiments, one type of cleaving reagent cleaves an amino acid or subset of amino acids that is different from an amino acid or subset of amino acids cleaved by another type of cleaving reagent in the reaction mixture.

### Polypeptide Analysis

In some aspects, the disclosure provides methods of polypeptide analysis (e.g., polypeptide sequencing). In some embodiments, a method of polypeptide analysis comprises: contacting a polypeptide with a reaction mixture described herein; monitoring a signal for signal pulses corresponding to interactions between one or more amino acid binding proteins and the polypeptide; and determining at least one chemical characteristic of the polypeptide based on a characteristic pattern in the signal.

A non-limiting example of polypeptide structure analysis by detecting single molecule binding interactions during a polypeptide degradation process is illustrated in FIG. 1A. An example signal trace is shown depicting different association (e.g., binding) events at times corresponding to changes in the signal. As shown, an association event between an amino acid recognizer and a terminal end of a polypeptide produces a change in magnitude of the signal that persists for a duration of time. Different association events are illustrated for different amino acids exposed at the terminal end of the polypeptide. As described herein, an amino acid that is “exposed” at the terminus of a polypeptide is an amino acid that is still attached to the polypeptide and that becomes the terminal amino acid upon removal of the prior terminal amino acid during degradation (e.g., either alone or along with one or more additional amino acids).

As generically depicted, the association events between amino acid recognizers and different types of amino acids at the terminal end of the polypeptide produce distinctive changes in the signal, referred to herein as a characteristic pattern, which may be used to determine chemical characteristics of the polypeptide. In some embodiments, a characteristic pattern corresponding to one type of terminal amino acid can be used to determine structural information for the terminal amino acid and one or more amino acids contiguous to the terminal amino acid. Accordingly, in some embodiments, a characteristic pattern corresponding to one type of terminal amino acid can be used to determine structural information for at least two (e.g., at least three, at least four, at least five, two, three, four, or between two and five) amino acids of a polypeptide.

In some embodiments, a transition from one characteristic pattern to another is indicative of amino acid cleavage. As used herein, in some embodiments, amino acid cleavage refers to the removal of at least one amino acid from a terminus of a polypeptide (e.g., the removal of at least one terminal amino acid from the polypeptide). In some embodiments, amino acid cleavage is determined by inference based on a time duration between characteristic patterns. In some embodiments, amino acid cleavage is determined by detecting a change in signal produced by association of a labeled cleaving reagent with an amino acid at the terminus of the polypeptide. As amino acids are sequentially cleaved from the terminus of the polypeptide during degradation, a series of changes in magnitude, or a series of signal pulses, is detected.

In some embodiments, signal data can be analyzed to extract signal pulse information by applying threshold levels to one or more parameters of the signal data. For example, in some embodiments, a threshold magnitude level may be applied to the signal data of a signal trace. In some embodiments, the threshold magnitude level is a minimum difference between a signal detected at a point in time and a baseline determined for a given set of data. In some embodiments, a signal pulse is assigned to each portion of the data that is indicative of a change in magnitude exceeding the threshold magnitude level and persisting for a duration of time. In some embodiments, a threshold time duration may be applied to a portion of the data that satisfies the threshold magnitude level to determine whether a signal pulse is assigned to that portion. For example, experimental artifacts may give rise to a change in magnitude exceeding the threshold magnitude level but that does not persist for a duration of time sufficient to assign a signal pulse with a desired confidence (e.g., transient association events which could be non-discriminatory for amino acid type, non-specific detection events such as diffusion into an observation region or reagent sticking within an observation region). Accordingly, in some embodiments, a signal pulse is extracted from signal data based on a threshold magnitude level and a threshold time duration.

In some embodiments, a peak in magnitude of a signal pulse is determined by averaging the magnitude detected over a duration of time that persists above the threshold magnitude level. It should be appreciated that, in some embodiments, a “signal pulse” as used herein can refer to a change in signal data that persists for a duration of time above a baseline (e.g., raw signal data), or to signal pulse information extracted therefrom (e.g., processed signal data).

In some embodiments, signal pulse information can be analyzed to identify different types of amino acids in a polypeptide based on different characteristic patterns in a series of signal pulses. For example, as shown in FIG. 1A, the signal pulse information is indicative of different types of amino acids at a terminal end of a polypeptide (e.g., arginine, leucine, isoleucine, phenylalanine). By way of example, the signal pulses detected at the earliest time points provide information indicative of (at least) arginine at the terminus of the polypeptide based on a first characteristic pattern, and the signal pulses detected at the latest time points provide information indicative of at least phenylalanine at the terminus of the polypeptide based on a second characteristic pattern.

In some embodiments, each signal pulse of a characteristic pattern comprises a pulse duration corresponding to an association event between an amino acid recognizer and an amino acid ligand. In some embodiments, the pulse duration is characteristic of a dissociation rate of binding. In some embodiments, each signal pulse of a characteristic pattern is separated from another signal pulse of the characteristic pattern by an interpulse duration. In some embodiments, the interpulse duration is characteristic of an association rate of binding. In some embodiments, a change in magnitude in a signal can be determined for a signal pulse based on a difference between baseline and the peak of a signal pulse. In some embodiments, a characteristic pattern is determined based on pulse duration. In some embodiments, a characteristic pattern is determined based on pulse duration and interpulse duration. In some embodiments, a characteristic pattern is determined based on any one or more of pulse duration, interpulse duration, and change in magnitude.

Accordingly, as illustrated by FIG. 1A, in some embodiments, polypeptide analysis is performed by detecting a series of signal pulses indicative of association of one or more amino acid recognizers with successive amino acids exposed at the terminus of a polypeptide in an ongoing degradation reaction. The series of signal pulses can be analyzed to determine characteristic patterns in the series of signal pulses, and the time course of characteristic patterns can be used to determine chemical characteristics throughout an amino acid sequence of the polypeptide.

As described herein, signal pulse information may be used to identify an amino acid based on a characteristic pattern in a series of signal pulses. In some embodiments, a characteristic pattern comprises a plurality of signal pulses, each signal pulse comprising a pulse duration. In some embodiments, the plurality of signal pulses may be characterized by a summary statistic (e.g., mean, median, time decay constant) of the distribution of pulse durations in a characteristic pattern. In some embodiments, the mean pulse duration of a characteristic pattern is between about 1 millisecond and about 10 seconds (e.g., between about 1 ms and about 1 s, between about 1 ms and about 100 ms, between about 1 ms and about 10 ms, between about 10 ms and about 10 s, between about 100 ms and about 10 s, between about 1 s and about 10 s, between about 10 ms and about 100 ms, or between about 100 ms and about 500 ms). In some embodiments, the mean pulse duration is between about 50 milliseconds and about 2 seconds, between about 50 milliseconds and about 500 milliseconds, or between about 500 milliseconds and about 2 seconds.

In some embodiments, different characteristic patterns corresponding to different types of amino acids in a single polypeptide may be distinguished from one another based on a statistically significant difference in the summary statistic. For example, in some embodiments, one characteristic pattern may be distinguishable from another characteristic pattern based on a difference in mean pulse duration of at least 10 milliseconds (e.g., between about 10 ms and about 10 s, between about 10 ms and about 1 s, between about 10 ms and about 100 ms, between about 100 ms and about 10 s, between about 1 s and about 10 s, or between about 100 ms and about 1 s). In some embodiments, the difference in mean pulse duration is at least 50 ms, at least 100 ms, at least 250 ms, at least 500 ms, or more. In some embodiments, the difference in mean pulse duration is between about 50 ms and about 1 s, between about 50 ms and about 500 ms, between about 50 ms and about 250 ms, between about 100 ms and about 500 ms, between about 250 ms and about 500 ms, or between about 500 ms and about 1 s. In some embodiments, the mean pulse duration of one characteristic pattern is different from the mean pulse duration of another characteristic pattern by about 10-25%, 25-50%, 50-75%, 75-100%, or more than 100%, for example by about 2-fold, 3-fold, 4-fold, 5-fold, or more. It should be appreciated that, in some embodiments, smaller differences in mean pulse duration between different characteristic patterns may require a greater number of pulse durations within each characteristic pattern to distinguish one from another with statistical confidence.

In some embodiments, a characteristic pattern generally refers to a plurality of association events between an amino acid of a polypeptide and a means for binding the amino acid (e.g., an amino acid recognition molecule). In some embodiments, a characteristic pattern comprises at least 10 association events (e.g., at least 25, at least 50, at least 75, at least 100, at least 250, at least 500, at least 1,000, or more, association events). In some embodiments, a characteristic pattern comprises between about 10 and about 1,000 association events (e.g., between about 10 and about 500 association events, between about 10 and about 250 association events, between about 10 and about 100 association events, or between about 50 and about 500 association events). In some embodiments, the plurality of association events is detected as a plurality of signal pulses.

In some embodiments, a characteristic pattern refers to a plurality of signal pulses which may be characterized by a summary statistic as described herein. In some embodiments, a characteristic pattern comprises at least 10 signal pulses (e.g., at least 25, at least 50, at least 75, at least 100, at least 250, at least 500, at least 1,000, or more, signal pulses). In some embodiments, a characteristic pattern comprises between about 10 and about 1,000 signal pulses (e.g., between about 10 and about 500 signal pulses, between about 10 and about 250 signal pulses, between about 10 and about 100 signal pulses, or between about 50 and about 500 signal pulses).

In some embodiments, a characteristic pattern refers to a plurality of association events between an amino acid recognition molecule and an amino acid of a polypeptide occurring over a time interval prior to removal of the amino acid (e.g., a cleavage event). In some embodiments, a characteristic pattern refers to a plurality of association events occurring over a time interval between two cleavage events (e.g., prior to removal of the amino acid and after removal of an amino acid previously exposed at the terminus). In some embodiments, the time interval of a characteristic pattern is between about 1 minute and about 30 minutes (e.g., between about 1 minute and about 20 minutes, between about 1 minute and 10 minutes, between about 5 minutes and about 20 minutes, between about 5 minutes and about 15 minutes, or between about 5 minutes and about 10 minutes).

In some embodiments, the series of signal pulses comprises a series of changes in magnitude of an optical signal over time. In some embodiments, the series of changes in the optical signal comprises a series of changes in luminescence produced during association events. In some embodiments, luminescence is produced by a detectable label associated with one or more reagents of a sequencing reaction. For example, in some embodiments, each of the one or more amino acid recognizers comprises a luminescent label. In some embodiments, a cleaving reagent comprises a luminescent label. Examples of luminescent labels and their use in accordance with the disclosure are provided herein.

In some embodiments, the series of signal pulses comprises a series of changes in magnitude of an electrical signal over time. In some embodiments, the series of changes in the electrical signal comprises a series of changes in conductance produced during association events. In some embodiments, conductivity is produced by a detectable label associated with one or more reagents of a sequencing reaction. For example, in some embodiments, each of the one or more amino acid recognizers comprises a conductivity label. Examples of conductivity labels and their use in accordance with the disclosure are provided elsewhere herein. Methods for identifying single molecules using conductivity labels have been described (see, e.g., U.S. Patent Publication No. 2017/0037462).

In some embodiments, the series of changes in conductance comprises a series of changes in conductance through a nanopore. For example, methods of evaluating receptor-ligand interactions using nanopores have been described (see, e.g., Thakur, A. K. & Movileanu, L. (2019) Nature Biotechnology 37(1)). The inventors have recognized and appreciated that such nanopores may be used to monitor polypeptide sequencing reactions in accordance with the disclosure. Accordingly, in some embodiments, the disclosure provides methods of polypeptide analysis comprising contacting a single polypeptide molecule with one or more amino acid recognizers described herein, where the single polypeptide molecule is immobilized to a nanopore. In some embodiments, the methods further comprise detecting a series of changes in conductance through the nanopore indicative of association of the one or more amino acid recognizers with successive amino acids exposed at a terminus of the single polypeptide while the single polypeptide is being degraded.

As described herein, in some embodiments, amino acid recognizers of the disclosure may be used to determine at least one chemical characteristic of a polypeptide. In some embodiments, determining at least one chemical characteristic comprises determining the type of amino acid that is present at a terminal end of a polypeptide and/or the types of amino acids that are present at one or more positions contiguous to the amino acid at the terminal end. In some embodiments, determining the type of amino acid comprises determining the actual amino acid identity, for example by determining which of the naturally-occurring 20 amino acids is present. In some embodiments, the type of amino acid is selected from alanine, arginine, asparagine, aspartic acid, cysteine, glutamine, glutamic acid, glycine, histidine, isoleucine, leucine, lysine, methionine, phenylalanine, proline, selenocysteine, serine, threonine, tryptophan, tyrosine, and valine.

In some embodiments, determining at least one chemical characteristic of a polypeptide comprises determining a subset of potential amino acids that can be present in the polypeptide. In some embodiments, this can be accomplished by determining that an amino acid is not one or more specific amino acids (and therefore could be any of the other amino acids). In some embodiments, this can be accomplished by determining which of a specified subset of amino acids (e.g., based on size, charge, hydrophobicity, post-translational modification, binding properties) could be in the polypeptide (e.g., using a recognizer that binds to a specified subset of two or more amino acids).

In some embodiments, determining at least one chemical characteristic of a polypeptide comprises determining that an amino acid comprises a post-translational modification. Non-limiting examples of post-translational modifications include acetylation (e.g., acetylated lysine), ADP-ribosylation, caspase cleavage, citrullination, formylation, N-linked glycosylation (e.g., glycosylated asparagine), O-linked glycosylation (e.g., glycosylated serine, glycosylated threonine), hydroxylation, methylation (e.g., methylated lysine, methylated arginine), myristoylation (e.g., myristoylated glycine), neddylation, nitration (e.g., nitrated tyrosine), chlorination (e.g., chlorinated tyrosine), oxidation/reduction (e.g., oxidized cysteine, oxidized methionine), palmitoylation (e.g., palmitoylated cysteine), phosphorylation, prenylation (e.g., prenylated cysteine), S-nitrosylation (e.g., S-nitrosylated cysteine, S-nitrosylated methionine), sulfation, sumoylation (e.g., sumoylated lysine), and ubiquitination (e.g., ubiquitinated lysine).

In some embodiments, determining at least one chemical characteristic of a polypeptide comprises determining that an amino acid comprises an arginine post-translational modification. For example, as described herein, amino acid recognizers of the disclosure are capable of distinguishing between different arginine modifications, including symmetric dimethylarginine (SDMA), asymmetric dimethylarginine (ADMA), and citrullinated arginine.

In some embodiments, determining at least one chemical characteristic of a polypeptide comprises determining that an amino acid comprises a phosphorylated side chain. For example, in some embodiments, determining at least one chemical characteristic of a polypeptide comprises determining that an amino acid comprises phosphorylated threonine (e.g., phospho-threonine). In some embodiments, determining at least one chemical characteristic of a polypeptide comprises determining that an amino acid comprises phosphorylated tyrosine (e.g., phospho-tyrosine). In some embodiments, determining at least one chemical characteristic of a polypeptide comprises determining that an amino acid comprises phosphorylated serine (e.g., phospho-serine).

In some embodiments, determining at least one chemical characteristic of a polypeptide comprises determining that an amino acid comprises a chemically modified variant, an unnatural amino acid, or a proteinogenic amino acid such as selenocysteine and pyrrolysine. Examples of unnatural amino acids include, without limitation, 2-naphthyl-alanine, statine, homoalanine, α-amino acid, β2-amino acid, β3-amino acid, γ-amino acid, 3-pyridyl-alanine, 4-fluorophenyl-alanine, cyclohexyl-alanine, N-alkyl amino acid, peptoid amino acid, homo-cysteine, penicillamine, 3-nitro-tyrosine, homo-phenyl-alanine, t-leucine, hydroxy-proline, 3-Abz, 5-F-tryptophan, and azabicyclo-[2.2.1]heptane.

In some embodiments, determining at least one chemical characteristic of a polypeptide comprises determining that an amino acid comprises an oxidative modification. For example, as described herein, amino acid recognizers of the disclosure are capable of distinguishing between oxidized methionine and its unmodified variant. In some embodiments, the oxidative modification comprises an oxidatively-damaged side chain of an amino acid. In some embodiments, the oxidatively-damaged side chain comprises a cysteine-derived product (e.g., disulfide, sulfinic acid, sulfonic acid, sulfenic acid, S-nitrosocysteine), a tyrosine-derived product (e.g., di-tyrosine, 3,4-dihydroxyphenylalanine, 3-chlorotyrosine, 3-nitrotyrosine), a histidine-derived product (e.g., 2-oxohistidine, 4-hydroxy-2-oxohistidine, di-histidine, asparagine, aspartic acid, urea), a methionine-derived product (e.g., sulfoxide, sulfone), a tryptophan-derived product (e.g., di-tryptophan, N-formylkynurenine, kynurenine, 2-oxo-tryptophan oxindolylalanine, 6-nitrotryptophan, hydroxytryptophan), a phenylalanine-derived product (e.g., meta-tyrosine, ortho-tyrosine), or a generic side-chain product (e.g., alcohol, hydroperoxide, aldehyde/ketone carbonyl). Examples of oxidatively damaged amino acids are known in the art, see, e.g., Hawkins, C. L., Davies, M. J. Detection, identification, and quantification of oxidative protein modifications. J Biol Chem. 2019 Dec. 20; 294(51):19683-19708.

In some embodiments, determining at least one chemical characteristic of a polypeptide comprises determining that an amino acid comprises a side chain characterized by one or more biochemical properties. For example, an amino acid may comprise a nonpolar aliphatic side chain, a positively charged side chain, a negatively charged side chain, a nonpolar aromatic side chain, or a polar uncharged side chain. Non-limiting examples of an amino acid comprising a nonpolar aliphatic side chain include alanine, glycine, valine, leucine, methionine, and isoleucine. Non-limiting examples of an amino acid comprising a positively charged side chain includes lysine, arginine, and histidine. Non-limiting examples of an amino acid comprising a negatively charged side chain include aspartate and glutamate. Non-limiting examples of an amino acid comprising a nonpolar, aromatic side chain include phenylalanine, tyrosine, and tryptophan. Non-limiting examples of an amino acid comprising a polar uncharged side chain include serine, threonine, cysteine, proline, asparagine, and glutamine.

In some embodiments, a protein or polypeptide can be digested into a plurality of smaller polypeptides and chemical characteristics can be determined for one or more of these smaller polypeptides. In some embodiments, a first terminus (e.g., N or C terminus) of a polypeptide is immobilized and the other terminus (e.g., the C or N terminus) is analyzed as described herein.

As used herein, sequencing a polypeptide refers to determining sequence information for a polypeptide. In some embodiments, this can involve determining the identity of each sequential amino acid for a portion (or all) of the polypeptide. However, in some embodiments, this can involve assessing the identity of a subset of amino acids within the polypeptide (e.g., and determining the relative position of one or more amino acid types without determining the identity of each amino acid in the polypeptide). However, in some embodiments, amino acid content information can be obtained from a polypeptide without directly determining the relative position of different types of amino acids in the polypeptide. The amino acid content alone may be used to infer the identity of the polypeptide that is present (e.g., by comparing the amino acid content to a database of polypeptide information and determining which polypeptide(s) have the same amino acid content).

In some embodiments, sequence information for a plurality of polypeptide products obtained from a longer polypeptide or protein (e.g., via enzymatic and/or chemical cleavage) can be analyzed to reconstruct or infer the sequence of the longer polypeptide or protein.

In some aspects, the polypeptide analysis described herein generates data indicating how a polypeptide interacts with a binding means while the polypeptide is being degraded by a cleaving means. As discussed above, the data can include a series of characteristic patterns corresponding to association events at a terminus of a polypeptide in between cleavage events at the terminus. In some embodiments, methods of polypeptide analysis described herein comprise contacting a single polypeptide molecule with a binding means and a cleaving means, where the binding means and the cleaving means are configured to achieve at least 10 association events prior to a cleavage event. In some embodiments, the means are configured to achieve the at least 10 association events between two cleavage events.

In some embodiments, a plurality of single-molecule sequencing reactions are performed in parallel in an array of sample wells. In some embodiments, an array comprises between about 10,000 and about 1,000,000 sample wells. The volume of a sample well may be between about 10⁻²¹ liters and about 10⁻¹⁵ liters, in some implementations. Because the sample well has a small volume, detection of single-molecule events may be possible as only about one polypeptide may be within a sample well at any given time. Statistically, some sample wells may not contain a single-molecule sequencing reaction and some may contain more than one single polypeptide molecule. However, an appreciable number of sample wells may each contain a single-molecule reaction (e.g., at least 30% in some embodiments), so that single-molecule analysis can be carried out in parallel for a large number of sample wells. In some embodiments, the binding means and the cleaving means are configured to achieve at least 10 association events prior to a cleavage event in at least 10% (e.g., 10-50%, more than 50%, 25-75%, at least 80%, or more) of the sample wells in which a single-molecule reaction is occurring. In some embodiments, the binding means and the cleaving means are configured to achieve at least 10 association events prior to a cleavage event for at least 50% (e.g., more than 50%, 50-75%, at least 80%, or more) of the amino acids of a polypeptide in a single-molecule reaction.

### Devices and Systems

Methods in accordance with the disclosure, in some aspects, may be performed using a system that permits single-molecule analysis. The system may include an integrated device and an instrument configured to interface with the integrated device. The integrated device may include an array of pixels, where individual pixels include a sample well and at least one photodetector. The sample wells of the integrated device may be formed on or through a surface of the integrated device and be configured to receive a sample placed on the surface of the integrated device. Collectively, the sample wells may be considered as an array of sample wells. The plurality of sample wells may have a suitable size and shape such that at least a portion of the sample wells receive a single sample (e.g., a single molecule, such as a polypeptide). In some embodiments, the number of samples within a sample well may be distributed among the sample wells of the integrated device such that some sample wells contain one sample while others contain zero, two or more samples.

Excitation light is provided to the integrated device from one or more light source external to the integrated device. Optical components of the integrated device may receive the excitation light from the light source and direct the light towards the array of sample wells of the integrated device and illuminate an illumination region within the sample well. In some embodiments, a sample well may have a configuration that allows for the sample to be retained in proximity to a surface of the sample well, which may ease delivery of excitation light to the sample and detection of emission light from the sample. A sample positioned within the illumination region may emit emission light in response to being illuminated by the excitation light. For example, the sample may be labeled with a fluorescent label, which emits light in response to achieving an excited state through the illumination of excitation light. Emission light emitted by a sample may then be detected by one or more photodetectors within a pixel corresponding to the sample well with the sample being analyzed. When performed across the array of sample wells, which may range in number between approximately 10,000 pixels to 1,000,000 pixels according to some embodiments, multiple samples can be analyzed in parallel.

The integrated device may include an optical system for receiving excitation light and directing the excitation light among the sample well array. The optical system may include one or more grating couplers configured to couple excitation light to other optical components of the integrated device and direct the excitation light to the other optical components. For example, the optical system may include optical components that direct the excitation light from the grating coupler(s) towards the sample well array. Such optical components may include optical splitters, optical combiners, and waveguides. In some embodiments, one or more optical splitters may couple excitation light from a grating coupler and deliver excitation light to at least one of the waveguides. According to some embodiments, the optical splitter may have a configuration that allows for delivery of excitation light to be substantially uniform across all the waveguides such that each of the waveguides receives a substantially similar amount of excitation light. Such embodiments may improve performance of the integrated device by improving the uniformity of excitation light received by sample wells of the integrated device. Examples of suitable components, e.g., for coupling excitation light to a sample well and/or directing emission light to a photodetector, to include in an integrated device are described in U.S. patent application Ser. No. 14/821,688, filed Aug. 7, 2015, titled “INTEGRATED DEVICE FOR PROBING, DETECTING AND ANALYZING MOLECULES,” and U.S. patent application Ser. No. 14/543,865, filed Nov. 17, 2014, titled “INTEGRATED DEVICE WITH EXTERNAL LIGHT SOURCE FOR PROBING, DETECTING, AND ANALYZING MOLECULES,” both of which are incorporated by reference in their entirety. Examples of suitable grating couplers and waveguides that may be implemented in the integrated device are described in U.S. patent application Ser. No. 15/844,403, filed Dec. 15, 2017, titled “OPTICAL COUPLER AND WAVEGUIDE SYSTEM,” which is incorporated by reference in its entirety.

Additional photonic structures may be positioned between the sample wells and the photodetectors and configured to reduce or prevent excitation light from reaching the photodetectors, which may otherwise contribute to signal noise in detecting emission light. In some embodiments, metal layers which may act as a circuitry for the integrated device, may also act as a spatial filter. Examples of suitable photonic structures may include spectral filters, a polarization filters, and spatial filters and are described in U.S. patent application Ser. No. 16/042,968, filed Jul. 23, 2018, titled “OPTICAL REJECTION PHOTONIC STRUCTURES,” and U.S. Provisional Patent Application No. 63/124,655, filed Dec. 11, 2020, titled “INTEGRATED CIRCUIT WITH IMPROVED CHARGE TRANSFER EFFICIENCY AND ASSOCIATED TECHNIQUES,” both of which are incorporated by reference in their entirety.

Components located off of the integrated device may be used to position and align an excitation source to the integrated device. Such components may include optical components including lenses, mirrors, prisms, windows, apertures, attenuators, and/or optical fibers. Additional mechanical components may be included in the instrument to allow for control of one or more alignment components. Such mechanical components may include actuators, stepper motors, and/or knobs. Examples of suitable excitation sources and alignment mechanisms are described in U.S. patent application Ser. No. 15/161,088, filed May 20, 2016, titled “PULSED LASER AND SYSTEM,” which is incorporated by reference in its entirety. Another example of a beam-steering module is described in U.S. patent application Ser. No. 15/842,720, filed Dec. 14, 2017, titled “COMPACT BEAM SHAPING AND STEERING ASSEMBLY,” which is incorporated herein by reference. Additional examples of suitable excitation sources are described in U.S. patent application Ser. No. 14/821,688, filed Aug. 7, 2015, titled “INTEGRATED DEVICE FOR PROBING, DETECTING AND ANALYZING MOLECULES,” which is incorporated by reference in its entirety.

The photodetector(s) positioned with individual pixels of the integrated device may be configured and positioned to detect emission light from the pixel's corresponding sample well. Examples of suitable photodetectors are described in U.S. patent application Ser. No. 14/821,656, filed Aug. 7, 2015, titled “INTEGRATED DEVICE FOR TEMPORAL BINNING OF RECEIVED PHOTONS,” which is incorporated by reference in its entirety. In some embodiments, a sample well and its respective photodetector(s) may be aligned along a common axis. In this manner, the photodetector(s) may overlap with the sample well within the pixel.

Characteristics of the detected emission light may provide an indication for identifying the label associated with the emission light. Such characteristics may include any suitable type of characteristic, including an arrival time of photons detected by a photodetector, an amount of photons accumulated over time by a photodetector, and/or a distribution of photons across two or more photodetectors. In some embodiments, such characteristics can be any one or a combination of two or more of luminescence lifetime, luminescence intensity, brightness, absorption spectra, emission spectra, luminescence quantum yield, wavelength (e.g., peak wavelength), and signal characteristics (e.g., pulse duration, interpulse durations, change in signal magnitude).

In some embodiments, a photodetector may have a configuration that allows for the detection of one or more timing characteristics associated with a sample's emission light (e.g., luminescence lifetime). The photodetector may detect a distribution of photon arrival times after a pulse of excitation light propagates through the integrated device, and the distribution of arrival times may provide an indication of a timing characteristic of the sample's emission light (e.g., a proxy for luminescence lifetime). In some embodiments, the one or more photodetectors provide an indication of the probability of emission light emitted by the label (e.g., luminescence intensity). In some embodiments, a plurality of photodetectors may be sized and arranged to capture a spatial distribution of the emission light. Output signals from the one or more photodetectors may then be used to distinguish a label from among a plurality of labels, where the plurality of labels may be used to identify a sample within the sample. In some embodiments, a sample may be excited by multiple excitation energies, and emission light and/or timing characteristics of the emission light emitted by the sample in response to the multiple excitation energies may distinguish a label from a plurality of labels.

In operation, parallel analyses of samples within the sample wells are carried out by exciting some or all of the samples within the wells using excitation light and detecting signals from sample emission with the photodetectors. Emission light from a sample may be detected by a corresponding photodetector and converted to at least one electrical signal. The electrical signals may be transmitted along conducting lines in the circuitry of the integrated device, which may be connected to an instrument interfaced with the integrated device. The electrical signals may be subsequently processed and/or analyzed. Processing or analyzing of electrical signals may occur on a suitable computing device either located on or off the instrument.

The instrument may include a user interface for controlling operation of the instrument and/or the integrated device. The user interface may be configured to allow a user to input information into the instrument, such as commands and/or settings used to control the functioning of the instrument. In some embodiments, the user interface may include buttons, switches, dials, and a microphone for voice commands. The user interface may allow a user to receive feedback on the performance of the instrument and/or integrated device, such as proper alignment and/or information obtained by readout signals from the photodetectors on the integrated device. In some embodiments, the user interface may provide feedback using a speaker to provide audible feedback. In some embodiments, the user interface may include indicator lights and/or a display screen for providing visual feedback to a user.

In some embodiments, the instrument may include a computer interface configured to connect with a computing device. The computer interface may be a USB interface, a FireWire interface, or any other suitable computer interface. A computing device may be any general purpose computer, such as a laptop or desktop computer. In some embodiments, a computing device may be a server (e.g., cloud-based server) accessible over a wireless network via a suitable computer interface. The computer interface may facilitate communication of information between the instrument and the computing device. Input information for controlling and/or configuring the instrument may be provided to the computing device and transmitted to the instrument via the computer interface. Output information generated by the instrument may be received by the computing device via the computer interface. Output information may include feedback about performance of the instrument, performance of the integrated device, and/or data generated from the readout signals of the photodetector.

In some embodiments, the instrument may include a processing device configured to analyze data received from one or more photodetectors of the integrated device and/or transmit control signals to the excitation source(s). In some embodiments, the processing device may comprise a general purpose processor, a specially-adapted processor (e.g., a central processing unit (CPU) such as one or more microprocessor or microcontroller cores, a field-programmable gate array (FPGA), an application-specific integrated circuit (ASIC), a custom integrated circuit, a digital signal processor (DSP), or a combination thereof). In some embodiments, the processing of data from one or more photodetectors may be performed by both a processing device of the instrument and an external computing device. In other embodiments, an external computing device may be omitted and processing of data from one or more photodetectors may be performed solely by a processing device of the integrated device.

According to some embodiments, the instrument that is configured to analyze samples based on luminescence emission characteristics may detect differences in luminescence lifetimes and/or intensities between different luminescent molecules, and/or differences between lifetimes and/or intensities of the same luminescent molecules in different environments. The inventors have recognized and appreciated that differences in luminescence emission lifetimes can be used to discern between the presence or absence of different luminescent molecules and/or to discern between different environments or conditions to which a luminescent molecule is subjected. In some cases, discerning luminescent molecules based on lifetime (rather than emission wavelength, for example) can simplify aspects of the system. As an example, wavelength-discriminating optics (such as wavelength filters, dedicated detectors for each wavelength, dedicated pulsed optical sources at different wavelengths, and/or diffractive optics) may be reduced in number or eliminated when discerning luminescent molecules based on lifetime. In some cases, a single pulsed optical source operating at a single characteristic wavelength may be used to excite different luminescent molecules that emit within a same wavelength region of the optical spectrum but have measurably different lifetimes. An analytic system that uses a single pulsed optical source, rather than multiple sources operating at different wavelengths, to excite and discern different luminescent molecules emitting in a same wavelength region can be less complex to operate and maintain, more compact, and may be manufactured at lower cost.

Although analytic systems based on luminescence lifetime analysis may have certain benefits, the amount of information obtained by an analytic system and/or detection accuracy may be increased by allowing for additional detection techniques. For example, some embodiments of the systems may additionally be configured to discern one or more properties of a sample based on luminescence wavelength and/or luminescence intensity. In some implementations, luminescence intensity may be used additionally or alternatively to distinguish between different luminescent labels. For example, some luminescent labels may emit at significantly different intensities or have a significant difference in their probabilities of excitation (e.g., at least a difference of about 35%) even though their decay rates may be similar. By referencing binned signals to measured excitation light, it may be possible to distinguish different luminescent labels based on intensity levels.

According to some embodiments, different luminescence lifetimes may be distinguished with a photodetector that is configured to time-bin luminescence emission events following excitation of a luminescent label. The time binning may occur during a single charge-accumulation cycle for the photodetector. A charge-accumulation cycle is an interval between read-out events during which photo-generated carriers are accumulated in bins of the time-binning photodetector. Examples of a time-binning photodetector are described in U.S. patent application Ser. No. 14/821,656, filed Aug. 7, 2015, titled “INTEGRATED DEVICE FOR TEMPORAL BINNING OF RECEIVED PHOTONS,” which is incorporated herein by reference. In some embodiments, a time-binning photodetector may generate charge carriers in a photon absorption/carrier generation region and directly transfer charge carriers to a charge carrier storage bin in a charge carrier storage region. In such embodiments, the time-binning photodetector may not include a carrier travel/capture region. Such a time-binning photodetector may be referred to as a “direct binning pixel.” Examples of time-binning photodetectors, including direct binning pixels, are described in U.S. patent application Ser. No. 15/852,571, filed Dec. 22, 2017, titled “INTEGRATED PHOTODETECTOR WITH DIRECT BINNING PIXEL,” which is incorporated herein by reference.

In some embodiments, different numbers of fluorophores of the same type may be linked to different reagents in a sample, so that each reagent may be identified based on luminescence intensity. For example, two fluorophores may be linked to a first labeled recognition molecule and four or more fluorophores may be linked to a second labeled recognition molecule. Because of the different numbers of fluorophores, there may be different excitation and fluorophore emission probabilities associated with the different recognition molecules. For example, there may be more emission events for the second labeled recognition molecule during a signal accumulation interval, so that the apparent intensity of the bins is significantly higher than for the first labeled recognition molecule.

The inventors have recognized and appreciated that distinguishing biological or chemical samples based on fluorophore decay rates and/or fluorophore intensities may enable a simplification of the optical excitation and detection systems. For example, optical excitation may be performed with a single-wavelength source (e.g., a source producing one characteristic wavelength rather than multiple sources or a source operating at multiple different characteristic wavelengths). Additionally, wavelength discriminating optics and filters may not be needed in the detection system. Also, a single photodetector may be used for each sample well to detect emission from different fluorophores. The phrase “characteristic wavelength” or “wavelength” is used to refer to a central or predominant wavelength within a limited bandwidth of radiation (e.g., a central or peak wavelength within a 20 nm bandwidth output by a pulsed optical source). In some cases, “characteristic wavelength” or “wavelength” may be used to refer to a peak wavelength within a total bandwidth of radiation output by a source.

According to an aspect of the present disclosure, an exemplary integrated device may be configured to perform single-molecule analysis in combination with an instrument as described above. It should be appreciated that the exemplary integrated device described herein is intended to be illustrative and that other integrated device configurations may be configured to perform any or all techniques described herein.

FIG. 1B illustrates a cross-sectional view of a pixel 1-112 of an integrated device 1-102. Pixel 1-112 includes a photodetection region, which may be a pinned photodiode (PPD), and a charge storage region, which may be a storage diode (SD0). In some embodiments, a photodetection region and charge storage regions may be formed in semiconductor material of a pixel by doping regions of the semiconductor material. For example, the photodetection region and charge storage regions can be formed using a same conductivity type (e.g., n-type doping or p-type doping).

During operation of pixel 1-112, excitation light may illuminate sample well 1-108 causing incident photons, including fluorescence emissions from a sample, to flow along the optical axis to photodetection region PPD. As shown in FIG. 1B, pixel 1-112 may include a waveguide 1-220 configured to optically (e.g., evanescently) couple excitation light from a grating coupler of the integrated device (not shown) to the sample well 1-108. In response, a sample in the sample well 1-108 may emit fluorescent light toward photodetection region PPD.

In some embodiments, pixel 1-112 may also include one or more photonic structures 1-230, which may include one or more optical rejection structures such as a spectral filter, a polarization filter, and/or a spatial filter. For example, the photonic structures 1-230 may be configured to reduce the amount of excitation light that reaches the photodetection region PPD and/or increase the amount of fluorescent emissions that reach the photodetection region PPD. Also shown in pixel 1-112, pixel 1-112 may include one or more metal layers 1-240, which may be configured as a filter and/or may carry control signals from a control circuit configured to control transfer gates, as described further herein.

In some embodiments, pixel 1-112 may include one or more transfer gates configured to control operation of pixel 1-112 by applying an electrical bias to one or more semiconductor regions of pixel 1-112 in response to one or more control signals. For example, when transfer gate ST0 induces a first electrical bias at the semiconductor region between photodetection region PPD and storage region SD0, a transfer path (e.g., charge transfer channel) may be formed in the semiconductor region. Charge carriers (e.g., photo-electrons) generated in photodetection region PPD by the incident photons may flow along the transfer path to storage region SD0. In some embodiments, the first electrical bias may be applied during a collection period during which charge carriers from the sample are selectively directed to storage region SD0. Alternatively, when transfer gate ST0 provides a second electrical bias at the semiconductor region between photodetection region PPD and storage region SD0, charge carriers from photodetection region PPD may be blocked from reaching storage region SD0 along the transfer path. In some embodiments, drain gate REJ may provide a channel to drain D to draw noise charge carriers generated in photodetection region PPD by the excitation light away from photodetection region PPD and storage region SD0, such as during a rejection period before fluorescent emission photons from the sample reach photodetection region PPD. In some embodiments, during a readout period, transfer gate ST0 may provide the second electrical bias and transfer gate TX0 may provide an electrical bias to cause charge carriers stored in storage region SD0 to flow to the readout region, which may be a floating diffusion (FD) region, for processing.

It should be appreciated that, in accordance with various embodiments, transfer gates described herein may include semiconductor material(s) and/or metal, and may include a gate of a field effect transistor (FET), a base of a bipolar junction transistor (BJT), and/or the like.

In some embodiments, operation of pixel 1-112 may include one or more collection sequences, each collection sequence including one or more rejection (e.g., drain) periods and one or more collection periods. In one example, a collection sequence performed in accordance with one or more pulses of an excitation light source may begin with a rejection period, such as to discard charge carriers generated in pixel 1-112 (e.g., in photodetection region PD) responsive to excitation photons from the light source. For instance, the excitation photons may arrive at pixel 1-112 prior to the arrival of fluorescence emission photons from the sample well. Transfer gates for the charge storage regions may be biased to have low conductivity in the charge transfer channels coupling the charge storage regions to the photodetection region, blocking transfer and accumulation of charge carriers in the charge storage regions. A drain gate for the drain region may be biased to have high conductivity in a drain channel between the photodetection region and the drain region, facilitating draining of charge carriers from the photodetection region to the drain region. Transfer gates for any charge storage regions coupled to the photodetection region may be biased to have low conductivity between the photodetection region and the charge storage regions, such that charge carriers are not transferred to or accumulated in the charge storage regions during the rejection period.

Following the rejection period, a collection period may occur in which charge carriers generated responsive to the incident photons are transferred to one or more charge storage regions. During the collection period, the incident photons may include fluorescent emission photons, resulting in accumulation of fluorescent emission charge carriers in the charge storage region(s). For instance, a transfer gate for one of the charge storage regions may be biased to have high conductivity between the photodetection region and the charge storage region, facilitating accumulation of charge carriers in the charge storage region. Any drain gates coupled to the photodetection region may be biased to have low conductivity between the photodetection region and the drain region such that charge carriers are not discarded during the collection period.

Some embodiments may include multiple rejection and/or collection periods in a collection sequence, such as a second rejection period and second collection period following a first rejection period and a collection period, where each pair of rejection and collection periods is conducted in response to a pulse of excitation light. In one example, charge carriers generated in the photodetection region during each collection period of a collection sequence (e.g., in response to a plurality of pulses of excitation light) may be aggregated in a single charge storage region. In some embodiments, charge carriers aggregated in the charge storage region may be read out for processing prior to the next collection sequence. Alternatively or additionally, in some embodiments, charge carriers aggregated in a first charge storage region during a first collection sequence may be transferred to a second charge storage region sequentially coupled to the first charge storage region and read out simultaneously with the next collection sequence. In some embodiments, a processing circuit configured to read out charge carriers from one or more pixels may be configured to determine one or more of luminescence intensity information, luminescence lifetime information, luminescence spectral information, and/or any other mode of luminescence information associated with performing techniques described herein.

In some embodiments, a first collection sequence may include transferring, to a charge storage region at a first time following each excitation pulse, charge carriers generated in the photodetection response in response to the excitation pulse, and a second collection sequence may include transferring, to the charge storage region at a second time following each excitation pulse, charge carriers generated in the photodetection response in response to the excitation pulse. For example, the number of charge carriers aggregated after the first and second times may indicate luminance lifetime information of the received light.

As described further herein, pixels of an integrated device may be controlled to perform one or more collection sequences using one or more control signals from a control circuit of the integrated circuit, such as by providing the control signal(s) to drain and/or transfer gates of the pixel(s) of the integrated circuit. In some embodiments, charge carriers may be read out from the FD region of each pixel during a readout pixel associated with each pixel and/or a row or column of pixels for processing. In some embodiments, FD regions of the pixels may be read out using correlated double sampling (CDS) techniques.

### Sequence Information

As described herein, in some embodiments, a cleaving reagent of the disclosure comprises an aminopeptidase having an amino acid sequence that shares a percentage of sequence identity with an amino acid sequence selected from Table 1. In some embodiments, a cleaving reagent comprises an aminopeptidase described herein and a tag sequence having an amino acid sequence that shares a percentage of sequence identity with an amino acid sequence selected from Table 2. For the purposes of comparing two or more amino acid sequences, the percentage of “sequence identity” between a first amino acid sequence and a second amino acid sequence (also referred to herein as “amino acid identity”) may be calculated by: dividing [the number of amino acid residues in the first amino acid sequence that are identical to the amino acid residues at the corresponding positions in the second amino acid sequence] by [the total number of amino acid residues in the first amino acid sequence] and multiplying by [100], in which each deletion, insertion, substitution or addition of an amino acid residue in the second amino acid sequence compared to the first amino acid sequence is considered as a difference at a single amino acid residue (position).

Alternatively, the degree of sequence identity between two amino acid sequences may be calculated using a known computer algorithm (e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA (1998) 85:2444, or by computerized implementations of algorithms available as Blast, Clustal Omega, or other sequence alignment algorithms) and, for example, using standard settings. Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.

Additionally, or alternatively, two or more sequences may be assessed for the identity between the sequences. The terms “identical” or percent “identity” in the context of two or more amino acid sequences, refer to two or more sequences or subsequences that are the same. Two sequences are “substantially identical” if two sequences have a specified percentage of amino acid residues that are the same (e.g., at least 80%, 85%, 90%, 95%, 96%, 97%, 98%, 99%, 99.5%, 99.6%, 99.7%, 99.8%, or 99.9% identical) over a specified region or over the entire sequence, when compared and aligned for maximum correspondence over a comparison window, or designated region as measured using one of the above sequence comparison algorithms or by manual alignment and visual inspection. Optionally, the identity exists over a region that is at least about 25, 50, 75, or 100 amino acids in length, or over a region that is 100 to 150, 150 to 200, 100 to 200, or 200 or more, amino acids in length.

Additionally, or alternatively, two or more sequences may be assessed for the alignment between the sequences. The terms “alignment” or “percent alignment” in the context of two or more amino acid sequences, refer to two or more sequences or subsequences that are the same. Two sequences are “substantially aligned” if two sequences have a specified percentage of amino acid residues that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the above sequence comparison algorithms or by manual alignment and visual inspection. Optionally, the alignment exists over a region that is at least about 25, 50, 75, or 100 amino acids in length, or over a region that is 100 to 150, 150 to 200, 100 to 200, or 200 or more amino acids in length.

TABLE 1
Non-limiting example sequences of aminopeptidases

| | | SEQ |
|---|---|---|
| | | ID |
| Name | Sequence | NO. |
| Pyrococcus | MEVRNMVDYELLKKVVEAPGVSGYEFLGIRDVVIEEIKDYVDEVKVDKLGNVI | 1 |
| horikoshii TET II | AHKKGEGPKVMIAAHMDQIGLMVTHIEKNGFLRVAPIGGVDPKTLIAQRFKVW | |
| Aminopeptidase | IDKGKFIYGVGASVPPHIQKPEDRKKAPDWDQIFIDIGAESKEEAEDMGVKIG | |
| (hTET II) | TVITWDGRLERLGKHRFVSIAFDDRIAVYTILEVAKQLKDAKADVYFVATVQE | |
| | EVGLRGARTSAFGIEPDYGFAIDVTIAADIPGTPEHKQVTHLGKGTAIKIMDR | |
| | SVICHPTIVRWLEELAKKHEIPYQLEILLGGGTDAGAIHLTKAGVPTGALSVP | |
| | ARYIHSNTEVVDERDVDATVELMTKALENIHELKI | |
| AP30 | MEVRNMVDYELLKKVVEAPGVSGYEFLGIRDVVIEEIKDYVDEVKVDKLGNVI | 2 |
| | AHKKGEGPKVMIAAHMDQIGLMVTHIEKNGFLRVAPIGGVDPKTLIAQRFKVW | |
| | IDKGKFIYGVGASVPPHIQKPEDRKKAPDWDQIFIDIGAESKEEAEDMGVKIG | |
| | TVITWDGRLERLGKHRFVSIAFDDRIAVYTILEVAKQLKDAKADVYFVATVQE | |
| | EVGLRGARTSAFGIEPDYGFAIDVTIAADIPGTPEHKQVTHLGKGTAIKIMDR | |
| | SVICHPTIVRWLEELAKKHEIPYQLEILLGGGTDAGAIHLTKAGVPTGALSVP | |
| | ARYIHSNTEVVDERDVDATVELMTKALENIHELKIGGSHHHHHHHHHHGGGSG | |
| | GGSGGGSGLNDFFEAQKIEWHEGGGSGGGSGGGSGLNDFFEAQKIEWHE | |
| Pyrococcus | MDLKGGESMVDWKLMQEIIEAPGVSGYEHLGIRDIVVDVLKEVADEVKVDKLG | 3 |
| horikoshii TET III | NVIAHFKGSSPRIMVAAHMDKIGVMVNHIDKDGYLHIVPIGGVLPETLVAQRI | |
| Aminopeptidase | RFFTEKGERYGVVGVLPPHLRRGQEDKGSKIDWDQIVVDVGASSKEEAEEMGF | |
| (hTET III) | RVGTVGEFAPNFTRLNEHRFATPYLDDRICLYAMIEAARQLGDHEADIYIVGS | |
| | VQEEVGLRGARVASYAINPEVGIAMDVTFAKQPHDKGKIVPELGKGPVMDVGP | |
| | NINPKLRAFADEVAKKYEIPLQVEPSPRPTGTDANMQINREGVATAVLSIPIR | |
| | YMHSQVELADARDVDNTIKLAKALLEELKPMDFTP | |
| AP37 | MDLKGGESMVDWKLMQEIIEAPGVSGYEHLGIRDIVVDVLKEVADEVKVDKLG | 4 |
| | NVIAHFKGSSPRIMVAAHMDKIGVMVNHIDKDGYLHIVPIGGVLPETLVAQRI | |
| | RFFTEKGERYGVVGVLPPHLRRGQEDKGSKIDWDQIVVDVGASSKEEAEEMGF | |
| | RVGTVGEFAPNFTRLNEHRFATPYLDDRICLYAMIEAARQLGDHEADIYIVGS | |
| | VQEEVGLRGARVASYAINPEVGIAMDVTFAKQPHDKGKIVPELGKGPVMDVGP | |
| | NINPKLRAFADEVAKKYEIPLQVEPSPRPTGTDANMQINREGVATAVLSIPIR | |
| | YMHSQVELADARDVDNTIKLAKALLEELKPMDFTPGHHHHHHHHHH | |
| Yersinia pestis | MTQQEYQNRRQALLAKMAPGSAAIIFAAPEATRSADSEYPYRQNSDFSYLTGF | 5 |
| Xaa-Prolyl | NEPEAVLILVKSDETHNHSVLFNRIRDLTAEIWFGRRLGQEAAPTKLAVDRAL | |
| aminopeptidase | PFDEINEQLYLLLNRLDVIYHAQGQYAYADNIVFAALEKLRHGFRKNLRAPAT | |
| (yPIP) | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQLE | |
| yPIP-6x His | MTQQEYQNRRQALLAKMAPGSAAIIFAAPEATRSADSEYPYRQNSDFSYLTGF | 6 |
| | NEPEAVLILVKSDETHNHSVLFNRIRDLTAEIWFGRRLGQEAAPTKLAVDRAL | |
| | PFDEINEQLYLLLNRLDVIYHAQGQYAYADNIVFAALEKLRHGFRKNLRAPAT | |
| | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQLEHHHHHH | |
| yPIP (truncated) | MTQQEYQNRRQALLAKMAPGSAAIIFAAPEATRSADSEYPYRQNSDFSYLTGF | 7 |
| | NEPEAVLILVKSDETHNHSVLFNRIRDLTAEIWFGRRLGQEAAPTKLAVDRAL | |
| | PFDEINEQLYLLLNRLDVIYHAQGQYAYADNIVFAALEKLRHGFRKNLRAPAT | |
| | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQ | |
| AP70 | MTQQEYQNRRQALLAKMAPGSAAIIFAAPEATRSADSEYPYRQNSDFSYLTGF | 8 |
| | NEPEAVLILVKSDETHNHSVLFNRIRDLTAEIWFGRRLGQEAAPTKLAVDRAL | |
| | PFDEINEQLYLLLNRLDVIYHAQGQYAYADNIVFAALEKLRHGFRKNLRAPAT | |
| | LTDWRPWLHEMRLFKSAEEIAVLRRAGEISALAHTRAMEKCRPGMFEYQLEGE | |
| | ILHEFTRHGARYPAYNTIVGGGENGCILHYTENECELRDGDLVLIDAGCEYRG | |
| | YAGDITRTFPVNGKFTPAQRAVYDIVLAAINKSLTLFRPGTSIREVTEEVVRI | |
| | MVVGLVELGILKGDIEQLIAEQAHRPFFMHGLSHWLGMDVHDVGDYGSSDRGR | |
| | ILEPGMVLTVEPGLYIAPDADVPPQYRGIGIRIEDDIVITATGNENLTASVVK | |
| | DPDDIEALMALNHAGENLYFQGGSHHHHHH | |
| L. pneumophila | MMVKQGVFMKTDQSKVKKLSDYKSLDYFVIHVDLQIDLSKKPVESKARLTVVP | 9 |
| M1 | NLNVDSHSNDLVLDGENMTLVSLQMNDNLLKENEYELTKDSLIIKNIPQNTPF | |
| Aminopeptidase | TIEMTSLLGENTDLFGLYETEGVALVKAESEGLRRVFYLPDRPDNLATYKTTI | |
| (Glu/Asp Specific) | IANQEDYPVLLSNGVLIEKKELPLGLHSVTWLDDVPKPSYLFALVAGNLQRSV | |
| | TYYQTKSGRELPIEFYVPPSATSKCDFAKEVLKEAMAWDERTFNLECALRQHM | |
| | VAGVDKYASGASEPTGLNLFNTENLFASPETKTDLGILRVLEVVAHEFFHYWS | |
| | GDRVTIRDWFNLPLKEGLTTFRAAMFREELFGTDLIRLLDGKNLDERAPRQSA | |
| | YTAVRSLYTAAAYEKSADIFRMMMLFIGKEPFIEAVAKFFKDNDGGAVTLEDF | |
| | IESISNSSGKDLRSFLSWFTESGIPELIVTDELNPDTKQYFLKIKTVNGRNRP | |
| | IPILMGLLDSSGAEIVADKLLIVDQEEIEFQFENIQTRPIPSLLRSFSAPVHM | |
| | KYEYSYQDLLLLMQFDTNLYNRCEAAKQLISALINDFCIGKKIELSPQFFAVY | |
| | KALLSDNSLNEWMLAELITLPSLEELIENQDKPDFEKLNEGRQLIQNALANEL | |
| | KTDFYNLLFRIQISGDDDKQKLKGFDLKQAGLRRLKSVCFSYLLNVDFEKTKE | |
| | KLILQFEDALGKNMTETALALSMLCEINCEEADVALEDYYHYWKNDPGAVNNW | |
| | FSIQALAHSPDVIERVKKLMRHGDFDLSNPNKVYALLGSFIKNPFGFHSVTGE | |
| | GYQLVADAIFDLDKINPTLAANLTEKFTYWDKYDVNRQAMMISTLKIIYSNAT | |
| | SSDVRTMAKKGLDKVKEDLPLPIHLTFHGGSTMQDRTAQLIADGNKENAYQLH | |
| E. coli methionine | MGTAISIKTPEDIEKMRVAGRLAAEVLEMIEPYVKPGVSTGELDRICNDYIVN | 10 |
| aminopeptidase | EQHAVSACLGYHGYPKSVCISINEVVCHGIPDDAKLLKDGDIVNIDVTVIKDG | |
| (Met specific) | FHGDTSKMFIVGKPTIMGERLCRITQESLYLALRMVKPGINLREIGAAIQKFV | |
| | EAEGFSVVREYCGHGIGRGFHEEPQVLHYDSRETNVVLKPGMTFTIEPMVNAG | |
| | KKEIRTMKDGWTVKTKDRSLSAQYEHTIVVTDNGCEILTLRKDDTIPAIISHD | |
| M. smegmatis | MGTLEANTNGPGSMLSRMPVSSRTVPFGDHETWVQVTTPENAQPHALPLIVLH | 11 |
| Proline | GGPGMAHNYVANIAALADETGRTVIHYDQVGCGNSTHLPDAPADFWTPQLFVD | |
| iminopeptidase | EFHAVCTALGIERYHVLGQSWGGMLGAEIAVRQPSGLVSLAICNSPASMRLWS | |
| (Pro specific) | EAAGDLRAQLPAETRAALDRHEAAGTITHPDYLQAAAEFYRRHVCRVVPTPQD | |
| | FADSVAQMEAEPTVYHTMNGPNEFHVVGTLGDWSVIDRLPDVTAPVLVIAGEH | |
| | DEATPKTWQPFVDHIPDVRSHVFPGTSHCTHLEKPEEFRAVVAQFLHQHDLAA | |
| | DARV | |
| P. furiosus | MDTEKLMKAGEIAKKVREKAIKLARPGMLLLELAESIEKMIMELGGKPAFPVN | 12 |
| methionine | LSINEIAAHYTPYKGDTTVLKEGDYLKIDVGVHIDGFIADTAVTVRVGMEEDE | |
| aminopeptidase | LMEAAKEALNAAISVARAGVEIKELGKAIENEIRKRGFKPIVNLSGHKIERYK | |
| | LHAGISIPNIYRPHDNYVLKEGDVFAIEPFATIGAGQVIEVPPTLIYMYVRDV | |
| | PVRVAQARFLLAKIKREYGTLPFAYRWLQNDMPEGQLKLALKTLEKAGAIYGY | |
| | PVLKEIRNGIVAQFEHTIIVEKDSVIVTQDMINKSTLE | |
| Aeromonas sobria | HMSSPLHYVLDGIHCEPHFFTVPLDHQQPDDEETITLFGRTLCRKDRLDDELP | 13 |
| Proline | WLLYLQGGPGFGAPRPSANGGWIKRALQEFRVLLLDQRGTGHSTPIHAELLAH | |
| aminopeptidase | LNPRQQADYLSHFRADSIVRDAELIREQLSPDHPWSLLGQSFGGFCSLTYLSL | |
| | FPDSLHEVYLTGGVAPIGRSADEVYRATYQRVADKNRAFFARFPHAQAIANRL | |
| | ATHLQRHDVRLPNGQRLTVEQLQQQGLDLGASGAFEELYYLLEDAFIGEKLNP | |
| | AFLYQVQAMQPFNTNPVFAILHELIYCEGAASHWAAERVRGEFPALAWAQGKD | |
| | FAFTGEMIFPWMFEQFRELIPLKEAAHLLAEKADWGPLYDPVQLARNKVPVAC | |
| | AVYAEDMYVEFDYSRETLKGLSNSRAWITNEYEHNGLRVDGEQILDRLIRLNR | |
| | DCLE | |
| Pyrococcus | MKERLEKLVKFMDENSIDRVFIAKPVNVYYFSGTSPLGGGYIIVDGDEATLYV | 14 |
| furiosus Proline | PELEYEMAKEESKLPVVKFKKFDEIYEILKNTETLGIEGTLSYSMVENFKEKS | |
| Aminopeptidase | NVKEFKKIDDVIKDLRIIKTKEEIEIIEKACEIADKAVMAAIEEITEGKRERE | |
| (X-/-Pro) | VAAKVEYLMKMNGAEKPAFDTIIASGHRSALPHGVASDKRIERGDLVVIDLGA | |
| | LYNHYNSDITRTIVVGSPNEKQREIYEIVLEAQKRAVEAAKPGMTAKELDSIA | |
| | REIIKEYGYGDYFIHSLGHGVGLEIHEWPRISQYDETVLKEGMVITIEPGIYI | |
| | PKLGGVRIEDTVLITENGAKRLTKTERELL | |
| Elizabethkingia | MIPITTPVGNFKVWTKRFGTNPKIKVLLLHGGPAMTHEYMECFETFFQREGFE | 15 |
| meningoseptica | FYEYDQLGSYYSDQPTDEKLWNIDRFVDEVEQVRKAIHADKENFYVLGNSWGG | |
| Proline | ILAMEYALKYQQNLKGLIVANMMASAPEYVKYAEVLSKQMKPEVLAEVRAIEA | |
| aminopeptidase | KKDYANPRYTELLFPNYYAQHICRLKEWPDALNRSLKHVNSTVYTLMQGPSEL | |
| | GMSSDARLAKWDIKNRLHEIATPTLMIGARYDTMDPKAMEEQSKLVQKGRYLY | |
| | CPNGSHLAMWDDQKVFMDGVIKFIKDVDTKSFN | |
| N. gonorrhoeae | MYEIKQPFHSGYLQVSEIHQIYWEESGNPDGVPVIFLHGGPGAGASPECRGFF | 16 |
| Proline | NPDVFRIVIIDQRGCGRSHPYACAEDNTTWDLVADIEKVREMLGIGKWLVFGG | |
| Iminopeptidase | SWGSTLSLAYAQTHPERVKGLVLRGIFLCRPSETAWLNEAGGVSRIYPEQWQK | |
| | FVAPIAENRRNRLIEAYHGLLFHQDEEVCLSAAKAWADWESYLIRFEPEGVDE | |
| | DAYASLAIARLENHYFVNGGWLQGDKAILNNIGKIRHIPTVIVQGRYDLCTPM | |
| | QSAWELSKAFPEAELRVVQAGHCAFDPPLADALVQAVEDILPRLL | |
| E. coli | MTQQPQAKYRHDYRAPDYQITDIDLTFDLDAQKTVVTAVSQAVRHGASDAPLR | 17 |
| Aminopeptidase N | LNGEDLKLVSVHINDEPWTAWKEEEGALVISNLPERFTLKIINEISPAANTAL | |
| (Zinc | EGLYQSGDALCTQCEAEGFRHITYYLDRPDVLARFTTKIIADKIKYPFLLSNG | |
| Metalloprotease) | NRVAQGELENGRHWVQWQDPFPKPCYLFALVAGDFDVLRDTFTTRSGREVALE | |
| | LYVDRGNLDRAPWAMTSLKNSMKWDEERFGLEYDLDIYMIVAVDFFNMGAMEN | |
| | KGLNIFNSKYVLARTDTATDKDYLDIERVIGHEYFHNWTGNRVTCRDWFQLSL | |
| | KEGLTVFRDQEFSSDLGSRAVNRINNVRTMRGLQFAEDASPMAHPIRPDMVIE | |
| | MNNFYTLTVYEKGAEVIRMIHTLLGEENFQKGMQLYFERHDGSAATCDDFVQA | |
| | MEDASNVDLSHFRRWYSQSGTPIVTVKDDYNPETEQYTLTISQRTPATPDQAE | |
| | KQPLHIPFAIELYDNEGKVIPLQKGGHPVNSVLNVTQAEQTFVFDNVYFQPVP | |
| | ALLCEFSAPVKLEYKWSDQQLTFLMRHARNDFSRWDAAQSLLATYIKLNVARH | |
| | QQGQPLSLPVHVADAFRAVLLDEKIDPALAAEILTLPSVNEMAELFDIIDPIA | |
| | IAEVREALTRTLATELADELLAIYNANYQSEYRVEHEDIAKRTLRNACLRFLA | |
| | FGETHLADVLVSKQFHEANNMTDALAALSAAVAAQLPCRDALMQEYDDKWHQN | |
| | GLVMDKWFILQATSPAANVLETVRGLLQHRSFTMSNPNRIRSLIGAFAGSNPA | |
| | AFHAEDGSGYLFLVEMLTDLNSRNPQVASRLIEPLIRLKRYDAKRQEKMRAAL | |
| | EQLKGLENLSGDLYEKITKALA | |
| P. falciparum M1 | PKIHYRKDYKPSGFIINQVTLNINIHDQETIVRSVLDMDISKHNVGEDLVFDG | 18 |
| aminopeptidase | VGLKINEISINNKKLVEGEEYTYDNEFLTIFSKFVPKSKFAFSSEVIIHPETN | |
| | YALTGLYKSKNIIVSQCEATGFRRITFFIDRPDMMAKYDVTVTADKEKYPVLL | |
| | SNGDKVNEFEIPGGRHGARFNDPPLKPCYLFAVVAGDLKHLSATYITKYTKKK | |
| | VELYVFSEEKYVSKLQWALECLKKSMAFDEDYFGLEYDLSRLNLVAVSDFNVG | |
| | AMENKGLNIFNANSLLASKKNSIDFSYARILTVVGHEYFHQYTGNRVTLRDWF | |
| | QLTLKEGLTVHRENLFSEEMTKTVTTRLSHVDLLRSVQFLEDSSPLSHPIRPE | |
| | SYVSMENFYTTTVYDKGSEVMRMYLTILGEEYYKKGFDIYIKKNDGNTATCED | |
| | FNYAMEQAYKMKKADNSANLNQYLLWFSQSGTPHVSFKYNYDAEKKQYSIHVN | |
| | QYTKPDENQKEKKPLFIPISVGLINPENGKEMISQTTLELTKESDTFVFNNIA | |
| | VKPIPSLFRGFSAPVYIEDQLTDEERILLLKYDSDAFVRYNSCTNIYMKQILM | |
| | NYNEFLKAKNEKLESFQLTPVNAQFIDAIKYLLEDPHADAGFKSYIVSLPQDR | |
| | YIINFVSNLDTDVLADTKEYIYKQIGDKLNDVYYKMFKSLEAKADDLTYFNDE | |
| | SHVDFDQMNMRTLRNTLLSLLSKAQYPNILNEIIEHSKSPYPSNWLTSLSVSA | |
| | YFDKYFELYDKTYKLSKDDELLLQEWLKTVSRSDRKDIYEILKKLENEVLKDS | |
| | KNPNDIRAVYLPFTNNLRRFHDISGKGYKLIAEVITKTDKFNPMVATQLCEPF | |
| | KLWNKLDTKRQELMLNEMNTMLQEPQISNNLKEYLLRLTNK | |
| Puromycin- | MWLAAAAPSLARRLLFLGPPPPPLLLLVFSRSSRRRLHSLGLAAMPEKRPFER | 19 |
| sensitive | LPADVSPINYSLCLKPDLLDFTFEGKLEAAAQVRQATNQIVMNCADIDIITAS | |
| aminopeptidase | YAPEGDEEIHATGFNYQNEDEKVTLSFPSTLQTGTGTLKIDFVGELNDKMKGF | |
| (NPEPPS) | YRSKYTTPSGEVRYAAVTQFEATDARRAFPCWDEPAIKATFDISLVVPKDRVA | |
| | LSNMNVIDRKPYPDDENLVEVKFARTPVMSTYLVAFVVGEYDFVETRSKDGVC | |
| | VRVYTPVGKAEQGKFALEVAAKTLPFYKDYFNVPYPLPKIDLIAIADFAAGAM | |
| | ENWGLVTYRETALLIDPKNSCSSSRQWVALVVGHELAHQWFGNLVTMEWWTHL | |
| | WLNEGFASWIEYLCVDHCFPEYDIWTQFVSADYTRAQELDALDNSHPIEVSVG | |
| | HPSEVDEIFDAISYSKGASVIRMLHDYIGDKDFKKGMNMYLTKFQQKNAATED | |
| | LWESLENASGKPIAAVMNTWTKQMGFPLIYVEAEQVEDDRLLRLSQKKFCAGG | |
| | SYVGEDCPQWMVPITISTSEDPNQAKLKILMDKPEMNVVLKNVKPDQWVKLNL | |
| | GTVGFYRTQYSSAMLESLLPGIRDLSLPPVDRLGLQNDLFSLARAGIISTVEV | |
| | LKVMEAFVNEPNYTVWSDLSCNLGILSTLLSHTDFYEEIQEFVKDVFSPIGER | |
| | LGWDPKPGEGHLDALLRGLVLGKLGKAGHKATLEEARRRFKDHVEGKQILSAD | |
| | LRSPVYLTVLKHGDGTTLDIMLKLHKQADMQEEKNRIERVLGATLLPDLIQKV | |
| | LTFALSEEVRPQDTVSVIGGVAGGSKHGRKAAWKFIKDNWEELYNRYQGGFLI | |
| | SRLIKLSVEGFAVDKMAGEVKAFFESHPAPSAERTIQQCCENILLNAAWLKRD | |
| | AESIHQYLLQRKASPPTV | |
| NPEPPS E366V | MWLAAAAPSLARRLLFLGPPPPPLLLLVFSRSSRRRLHSLGLAAMPEKRPFER | 20 |
| | LPADVSPINYSLCLKPDLLDFTFEGKLEAAAQVRQATNQIVMNCADIDIITAS | |
| | YAPEGDEEIHATGFNYQNEDEKVTLSFPSTLQTGTGTLKIDFVGELNDKMKGF | |
| | YRSKYTTPSGEVRYAAVTQFEATDARRAFPCWDEPAIKATFDISLVVPKDRVA | |
| | LSNMNVIDRKPYPDDENLVEVKFARTPVMSTYLVAFVVGEYDFVETRSKDGVC | |
| | VRVYTPVGKAEQGKFALEVAAKTLPFYKDYFNVPYPLPKIDLIAIADFAAGAM | |
| | ENWGLVTYRETALLIDPKNSCSSSRQWVALVVGHVLAHQWFGNLVTMEWWTHL | |
| | WLNEGFASWIEYLCVDHCFPEYDIWTQFVSADYTRAQELDALDNSHPIEVSVG | |
| | HPSEVDEIFDAISYSKGASVIRMLHDYIGDKDFKKGMNMYLTKFQQKNAATED | |
| | LWESLENASGKPIAAVMNTWTKQMGFPLIYVEAEQVEDDRLLRLSQKKFCAGG | |
| | SYVGEDCPQWMVPITISTSEDPNQAKLKILMDKPEMNVVLKNVKPDQWVKLNL | |
| | GTVGFYRTQYSSAMLESLLPGIRDLSLPPVDRLGLQNDLFSLARAGIISTVEV | |
| | LKVMEAFVNEPNYTVWSDLSCNLGILSTLLSHTDFYEEIQEFVKDVFSPIGER | |
| | LGWDPKPGEGHLDALLRGLVLGKLGKAGHKATLEEARRRFKDHVEGKQILSAD | |
| | LRSPVYLTVLKHGDGTTLDIMLKLHKQADMQEEKNRIERVLGATLLPDLIQKV | |
| | LTFALSEEVRPQDTVSVIGGVAGGSKHGRKAAWKFIKDNWEELYNRYQGGFLI | |
| | SRLIKLSVEGFAVDKMAGEVKAFFESHPAPSAERTIQQCCENILLNAAWLKRD | |
| | AESIHQYLLQRKASPPTV | |
| Francisella | MIYEFVMTDPKIKYLKDYKPSNYLIDETHLIFELDESKTRVTANLYIVANREN | 21 |
| tularensis | RENNTLVLDGVELKLLSIKLNNKHLSPAEFAVNENQLIINNVPEKFVLQTVVE | |
| Aminopeptidase N | INPSANTSLEGLYKSGDVFSTQCEATGFRKITYYLDRPDVMAAFTVKIIADKK | |
| | KYPIILSNGDKIDSGDISDNQHFAVWKDPFKKPCYLFALVAGDLASIKDTYIT | |
| | KSQRKVSLEIYAFKQDIDKCHYAMQAVKDSMKWDEDRFGLEYDLDTFMIVAVP | |
| | DFNAGAMENKGLNIFNTKYIMASNKTATDKDFELVQSVVGHEYFHNWTGDRVT | |
| | CRDWFQLSLKEGLTVFRDQEFTSDLNSRDVKRIDDVRIIRSAQFAEDASPMSH | |
| | PIRPESYIEMNNFYTVTVYNKGAEIIRMIHTLLGEEGFQKGMKLYFERHDGQA | |
| | VTCDDFVNAMADANNRDFSLFKRWYAQSGTPNIKVSENYDASSQTYSLTLEQT | |
| | TLPTADQKEKQALHIPVKMGLINPEGKNIAEQVIELKEQKQTYTFENIAAKPV | |
| | ASLFRDFSAPVKVEHKRSEKDLLHIVKYDNNAFNRWDSLQQIATNIILNNADL | |
| | NDEFLNAFKSILHDKDLDKALISNALLIPIESTIAEAMRVIMVDDIVLSRKNV | |
| | VNQLADKLKDDWLAVYQQCNDNKPYSLSAEQIAKRKLKGVCLSYLMNASDQKV | |
| | GTDLAQQLFDNADNMTDQQTAFTELLKSNDKQVRDNAINEFYNRWRHEDLVVN | |
| | KWLLSQAQISHESALDIVKGLVNHPAYNPKNPNKVYSLIGGFGANFLQYHCKD | |
| | GLGYAFMADTVLALDKFNHQVAARMARNLMSWKRYDSDRQAMMKNALEKIKAS | |
| | NPSKNVFEIVSKSLES | |
| T. aquaticus | MDAFTENLNKLAELAIRVGLNLEEGQEIVATAPIEAVDFVRLLAEKAYENGAS | 22 |
| Aminopeptidase T | LFTVLYGDNLIARKRLALVPEAHLDRAPAWLYEGMAKAFHEGAARLAVSGNDP | |
| | KALEGLPPERVGRAQQAQSRAYRPTLSAITEFVTNWTIVPFAHPGWAKAVFPG | |
| | LPEEEAVQRLWQAIFQATRVDQEDPVAAWEAHNRVLHAKVAFLNEKRFHALHF | |
| | QGPGTDLTVGLAEGHLWQGGATPTKKGRLCNPNLPTEEVFTAPHRERVEGVVR | |
| | ASRPLALSGQLVEGLWARFEGGVAVEVGAEKGEEVLKKLLDTDEGARRLGEVA | |
| | LVPADNPIAKTGLVFFDTLFDENAASHIAFGQAYAENLEGRPSGEEFRRRGGN | |
| | ESMVHVDWMIGSEEVDVDGLLEDGTRVPLMRRGRWVI | |
| Bacillus | MAKLDETLTMLKALTDAKGVPGNEREARDVMKTYIAPYADEVTTDGLGSLIAK | 23 |
| stearothermophilus | KEGKSGGPKVMIAGHLDEVGFMVTQIDDKGFIRFQTLGGWWSQVMLAQRVTIV | |
| Peptidase M28 | TKKGDITGVIGSKPPHILPSEARKKPVEIKDMFIDIGATSREEAMEWGVRPGD | |
| | MIVPYFEFTVLNNEKMLLAKAWDNRIGCAVAIDVLKQLKGVDHPNTVYGVGTV | |
| | QEEVGLRGARTAAQFIQPDIAFAVDVGIAGDTPGVSEKEAMGKLGAGPHIVLY | |
| | DATMVSHRGLREFVIEVAEELNIPHHFDAMPGVGTDAGAIHLTGIGVPSLTIA | |
| | IPTRYIHSHAAILHRDDYENTVKLLVEVIKRLDADKVKQLTFDE | |
| Vibrio cholera | MEDKVWISMGADAVGSLNPALSESLLPHSFASGSQVWIGEVAIDELAELSHTM | 24 |
| Aminopeptidase | HEQHNRCGGYMVHTSAQGAMAALMMPESIANFTIPAPSQQDLVNAWLPQVSAD | |
| | QITNTIRALSSFNNRFYTTTSGAQASDWLANEWRSLISSLPGSRIEQIKHSGY | |
| | NQKSVVLTIQGSEKPDEWVIVGGHLDSTLGSHTNEQSIAPGADDDASGIASLS | |
| | EIIRVLRDNNFRPKRSVALMAYAAEEVGLRGSQDLANQYKAQGKKVVSVLQLD | |
| | MTNYRGSAEDIVFITDYTDSNLTQFLTTLIDEYLPELTYGYDRCGYACSDHAS | |
| | WHKAGFSAAMPFESKFKDYNPKIHTSQDTLANSDPTGNHAVKFTKLGLAYVIE | |
| | MANAGSSQVPDDSVLQDGTAKINLSGARGTQKRFTFELSQSKPLTIQTYGGSG | |
| | DVDLYVKYGSAPSKSNWDCRPYQNGNRETCSFNNAQPGIYHVMLDGYTNYNDV | |
| | ALKASTQ | |
| Photobacterium | MEDKVWISIGSDASQTVKSVMQSNARSLLPESLASNGPVWVGQVDYSQLAELS | 25 |
| halotolerans | HHMHEDHQRCGGYMVHSSPESAIAASNMPQSLVAFSIPEISQQDTVNAWLPQV | |
| Aminopeptidase | NSQAITGTITSLTSFINRFYTTTSGAQASDWLANEWRSLSASLPNASVRQVSH | |
| | FGYNQKSVVLTITGSEKPDEWIVLGGHLDSTIGSHTNEQSVAPGADDDASGIA | |
| | SVTEIIRVLSENNFQPKRSIAFMAYAAEEVGLRGSQDLANQYKAEGKQVISAL | |
| | QLDMTNYKGSVEDIVFITDYTDSNLTTFLSQLVDEYLPSLTYGFDTCGYACSD | |
| | HASWHKAGFSAAMPFEAKFNDYNPMIHTPNDTLQNSDPTASHAVKFTKLGLAY | |
| | AIEMASTTGGTPPPTGNVLKDGVPVNGLSGATGSQVHYSFELPAQKNLQISTA | |
| | GGSGDVDLYVSFGSEATKQNWDCRPYRNGNNEVCTFAGATPGTYSIMLDGYRQ | |
| | FSGVTLKASTQ | |
| Yersinia pestis | MTQQPQAKYRHDYRAPDYTITDIDLDFALDAQKTTVTAVSKVKRQGTDVTPLI | 26 |
| Aminopeptidase N | LNGEDLTLISVSVDGQAWPHYRQQDNTLVIEQLPADFTLTIVNDIHPATNSAL | |
| | EGLYLSGEALCTQCEAEGFRHITYYLDRPDVLARFTTRIVADKSRYPYLLSNG | |
| | NRVGQGELDDGRHWVKWEDPFPKPSYLFALVAGDFDVLQDKFITRSGREVALE | |
| | IFVDRGNLDRADWAMTSLKNSMKWDETRFGLEYDLDIYMIVAVDFFNMGAMEN | |
| | KGLNVFNSKYVLAKAETATDKDYLNIEAVIGHEYFHNWTGNRVTCRDWFQLSL | |
| | KEGLTVFRDQEFSSDLGSRSVNRIENVRVMRAAQFAEDASPMAHAIRPDKVIE | |
| | MNNFYTLTVYEKGSEVIRMMHTLLGEQQFQAGMRLYFERHDGSAATCDDFVQA | |
| | MEDVSNVDLSLFRRWYSQSGTPLLTVHDDYDVEKQQYHLFVSQKTLPTADQPE | |
| | KLPLHIPLDIELYDSKGNVIPLQHNGLPVHHVLNVTEAEQTFTFDNVAQKPIP | |
| | SLLREFSAPVKLDYPYSDQQLTFLMQHARNEFSRWDAAQSLLATYIKLNVAKY | |
| | QQQQPLSLPAHVADAFRAILLDEHLDPALAAQILTLPSENEMAELFTTIDPQA | |
| | ISTVHEAITRCLAQELSDELLAVYVANMTPVYRIEHGDIAKRALRNTCLNYLA | |
| | FGDEEFANKLVSLQYHQADNMTDSLAALAAAVAAQLPCRDELLAAFDVRWNHD | |
| | GLVMDKWFALQATSPAANVLVQVRTLLKHPAFSLSNPNRTRSLIGSFASGNPA | |
| | AFHAADGSGYQFLVEILSDLNTRNPQVAARLIEPLIRLKRYDAGRQALMRKAL | |
| | EQLKTLDNLSGDLYEKITKALAA | |
| Vibrio | MEEKVWISIGGDATQTALRSGAQSLLPENLINQTSVWVGQVPVSELATLSHEM | 27 |
| anguillarum | HENHQRCGGYMVHPSAQSAMSVSAMPLNLNAFSAPEITQQTTVNAWLPSVSAQ | |
| Aminopeptidase | QITSTITTLTQFKNRFYTTSTGAQASNWIADHWRSLSASLPASKVEQITHSGY | |
| | NQKSVMLTITGSEKPDEWVVIGGHLDSTLGSRTNESSIAPGADDDASGIAGVT | |
| | EIIRLLSEQNFRPKRSIAFMAYAAEEVGLRGSQDLANRFKAEGKKVMSVMQLD | |
| | MTNYQGSREDIVFITDYTDSNFTQYLTQLLDEYLPSLTYGFDTCGYACSDHAS | |
| | WHAVGYPAAMPFESKFNDYNPNIHSPQDTLQNSDPTGFHAVKFTKLGLAYVVE | |
| | MGNASTPPTPSNQLKNGVPVNGLSASRNSKTWYQFELQEAGNLSIVLSGGSGD | |
| | ADLYVKYQTDADLQQYDCRPYRSGNNETCQFSNAQPGRYSILLHGYNNYSNAS | |
| | LVANAQ | |
| Salinivibrio | MEDKKVWISIGADAQQTALSSGAQPLLAQSVAHNGQAWIGEVSESELAALSHE | 28 |
| spYCSC6 | MHENHHRCGGYIVHSSAQSAMAASNMPLSRASFIAPAISQQALVTPWISQIDS | |
| Aminopeptidase | ALIVNTIDRLTDFPNRFYTTTSGAQASDWIKQRWQSLSAGLAGASVTQISHSG | |
| | YNQASVMLTIEGSESPDEWVVVGGHLDSTIGSRTNEQSIAPGADDDASGIAAV | |
| | TEVIRVLAQNNFQPKRSIAFVAYAAEEVGLRGSQDVANQFKQAGKDVRGVLQL | |
| | DMTNYQGSAEDIVFITDYTDNQLTQYLTQLLDEYLPTLNYGFDTCGYACSDHA | |
| | SWHQVGYPAAMPFEAKFNDYNPNIHTPQDTLANSDSEGAHAAKFTKLGLAYTV | |
| | ELANADSSPNPGNELKLGEPINGLSGARGNEKYFNYRLDQSGELVIRTYGGSG | |
| | DVDLYVKANGDVSTGNWDCRPYRSGNDEVCRFDNATPGNYAVMLRGYRTYDNV | |
| | SLIVE | |
| Vibrio | MPPITQQATVTAWLPQVDASQITGTISSLESFTNRFYTTTSGAQASDWIASEW | 29 |
| proteolyticus | QALSASLPNASVKQVSHSGYNQKSVVMTITGSEAPDEWIVIGGHLDSTIGSHT | |
| Aminopeptidase I | NEQSVAPGADDDASGIAAVTEVIRVLSENNFQPKRSIAFMAYAAEEVGLRGSQ | |
| | DLANQYKSEGKNVVSALQLDMTNYKGSAQDVVFITDYTDSNFTQYLTQLMDEY | |
| | LPSLTYGFDTCGYACSDHASWHNAGYPAAMPFESKFNDYNPRIHTTQDTLANS | |
| | DPTGSHAKKFTQLGLAYAIEMGSATGDTPTPGNQLE | |
| Vibrio | MPPITQQATVTAWLPQVDASQITGTISSLESFTNRFYTTTSGAQASDWIASEW | 30 |
| proteolyticus | QFLSASLPNASVKQVSHSGYNQKSVVMTITGSEAPDEWIVIGGHLDSTIGSHT | |
| Aminopeptidase I | NEQSVAPGADDDASGIAAVTEVIRVLSENNFQPKRSIAFMAYAAEEVGLRGSQ | |
| (A55F) | DLANQYKSEGKNVVSALQLDMTNYKGSAQDVVFITDYTDSNFTQYLTQLMDEY | |
| | LPSLTYGFDTCGYACSDHASWHNAGYPAAMPFESKFNDYNPRIHTTQDTLANS | |
| | DPTGSHAKKFTQLGLAYAIEMGSATGDTPTPGNQLE | |
| P. furiosus | MVDWELMKKIIESPGVSGYEHLGIRDLVVDILKDVADEVKIDKLGNVIAHFKG | 31 |
| Aminopeptidase I | SAPKVMVAAHMDKIGLMVNHIDKDGYLRVVPIGGVLPETLIAQKIRFFTEKGE | |
| | RYGVVGVLPPHLRREAKDQGGKIDWDSIIVDVGASSREEAEEMGFRIGTIGEF | |
| | APNFTRLSEHRFATPYLDDRICLYAMIEAARQLGEHEADIYIVASVQEEIGLR | |
| | GARVASFAIDPEVGIAMDVTFAKQPNDKGKIVPELGKGPVMDVGPNINPKLRQ | |
| | FADEVAKKYEIPLQVEPSPRPTGTDANVMQINREGVATAVLSIPIRYMHSQVE | |
| | LADARDVDNTIKLAKALLEELKPMDFTPLE | |

TABLE 2
Non-limiting examples of tag sequences.

| | | SEQ |
|---|---|---|
| | | ID |
| Description | Sequence | NO: |
| 6x His-tag | HHHHHH | 32 |
| 6x His-tag with linker | GGSHHHHHH | 33 |
| 10x His-tag | HHHHHHHHHH | 34 |
| 10x His-tag with | GHHHHHHHHHH | 35 |
| linker | | |
| Biotinylation tag | GLNDFFEAQKIEWHE | 36 |
| Biotinylation tag with | GGGSGLNDFFEAQKIEWHE | 37 |
| linker | | |
| Biotinylation tag with | GGGGGGSGGGSGLNDFFEAQKIEWHE | 38 |
| linker | | |
| Bis-biotinylation tag | GGGSGGGSGGGSGLNDFFEAQKIEWHE | 39 |
| with linkers | GGGSGGGSGGGSGLNDFFEAQKIEWHE | |
| Bis-biotinylation tag | GSGGGSGGGSGGGSGLNDFFEAQKIEW | 40 |
| with linkers | HEGGGSGGGSGGGSGLNDFFEAQKIEW | |
| | HE | |
| His/biotinylation tags | GHHHHHHHHHHGGGSGGGSGGGSGLND | 41 |
| with linkers | FFEAQKIEWHE | |
| His/bis-biotinylation | GHHHHHHHHHHGGGSGGGSGGGSGLND | 42 |
| tags with linkers | FFEAQKIEWHEGGGSGGGSGGGSGLND | |
| | FFEAQKIEWHE | |
| His/bis-biotinylation | GGSHHHHHHHHHHGGGSGGGSGGGSGL | 43 |
| tags with linkers | NDFFEAQKIEWHEGGGSGGGSGGGSGL | |
| | NDFFEAQKIEWHE | |
| His/bis-biotinylation | GSHHHHHHHHHHGGGSGGGSGGGSGLN | 44 |
| tags with linkers | DFFEAQKIEWHEGGGSGGGSGGGSGLN | |
| | DFFEAQKIEWHE | |
| Bis-biotinylation/His | GGGSGGGSGGGSGLNDFFEAQKIEWHE | 45 |
| tags with linkers | GGGSGGGSGGGSGLNDFFEAQKIEWHE | |
| | GHHHHHH | |

### EXAMPLES

### Example 1. Evaluation of Pyrococcus horikoshii TET II Aminopeptidases

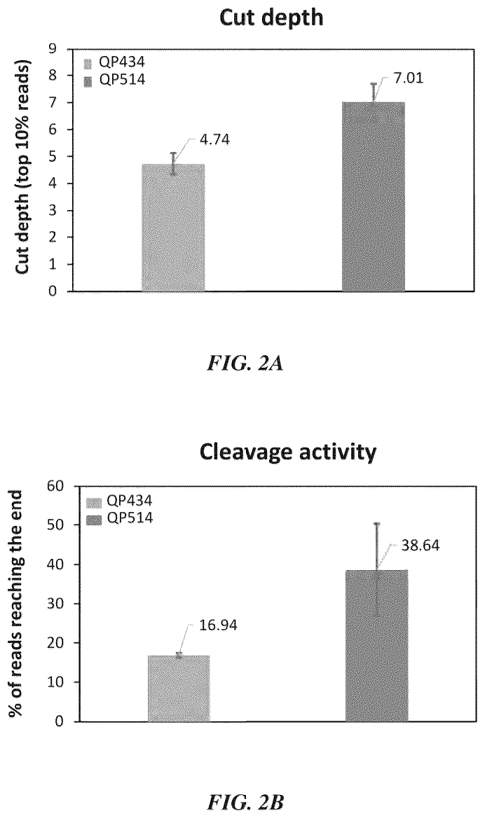

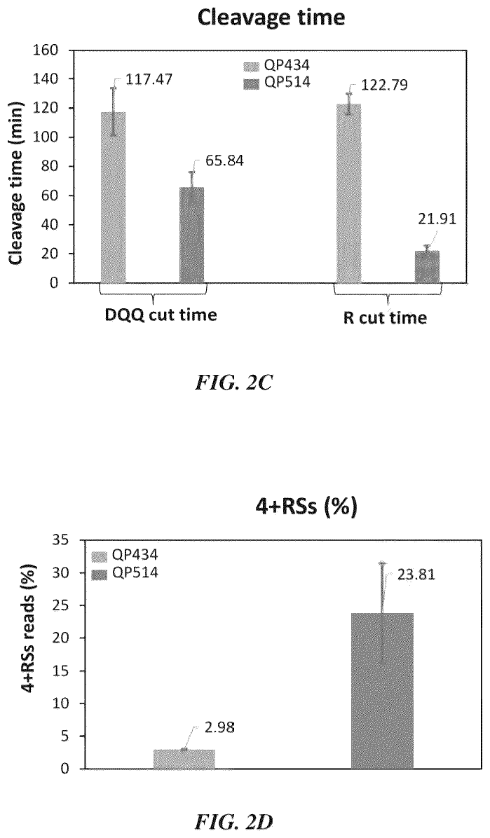

Cutting performance was evaluated with peptide substrates extended at the C-terminal end by the addition of a tripeptide DDD motif. FIGS. 2A-2D show TET aminopeptidase performance improvement by extending the C terminal of the peptide with DDD motif. The bar charts in FIGS. 2A-2D show the beneficial effect of extension of C terminal with DDD peptide motif on kinetics of hTETII/PfuTET combination (at 1 μM/40 μM) with QP514 (DQQRLIFAYPDDD) (SEQ ID NO: 49) and control QP434 (DQQRLIFAG, without the DDD motif) peptides. Plots are shown for cut depth (FIG. 2A), cleavage activity (% of reads reaching the last visible RS) (FIG. 2B), time taken to cleave DQQ motif and R residue (FIG. 2C), and % of 4+RSs (FIG. 2D).

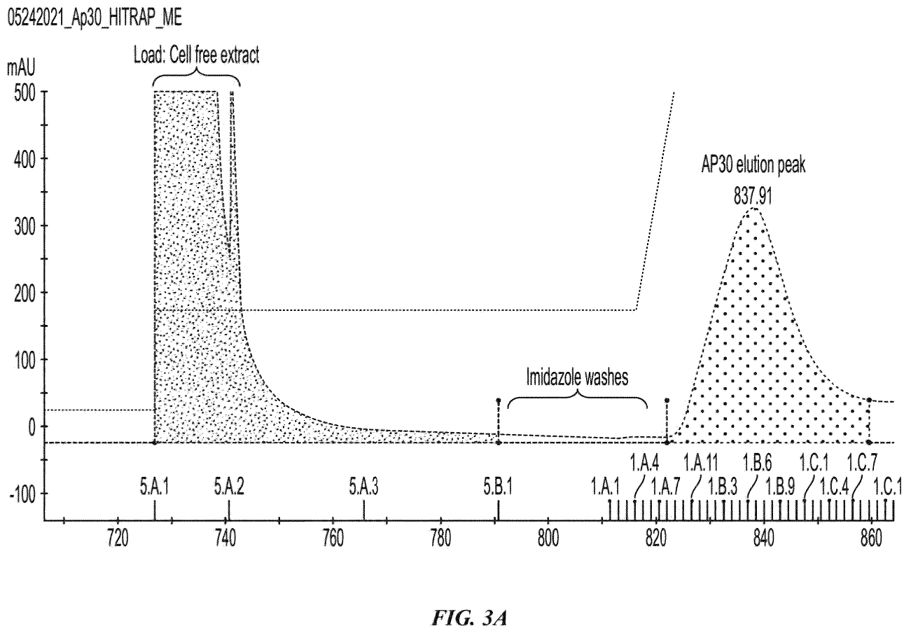

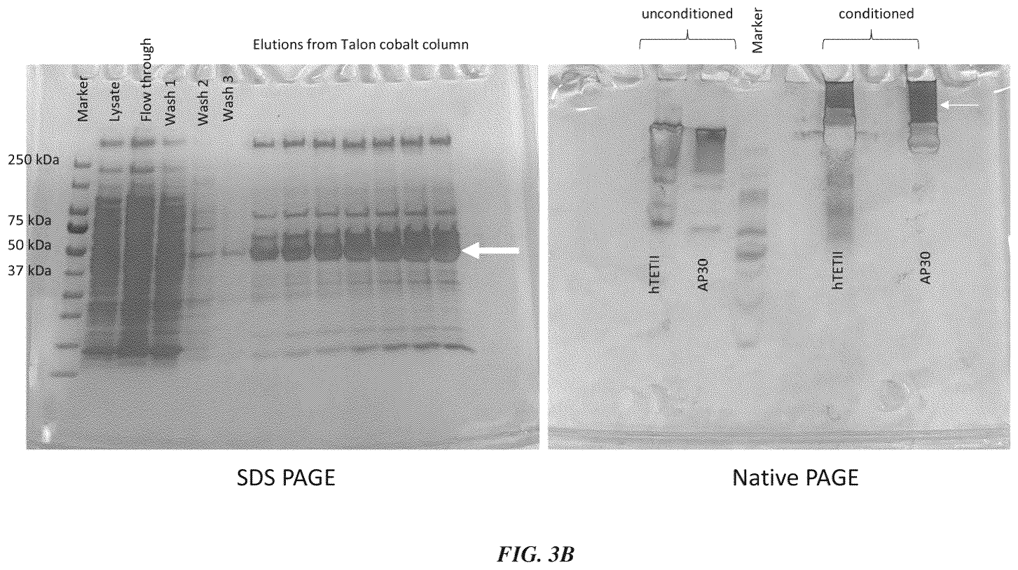

Cutting performance was evaluated for AP30, a cleaving reagent comprising Pyrococcus horikoshii TET II Aminopeptidase (hTET II) and a C-terminal tag. AP30 was expressed in E. coli, and the cell free extract was prepared after the cell lysis and resolved in talon affinity column in AKTA. FIG. 3A shows an example chromatogram showing AP30 separation and the eluted peak.

FIG. 3B (left image) shows Talon affinity column purification fractions resolved on SDS PAGE gel to show AP30 enrichment. The eluted peak fractions with majority monomer (45.83 kDa) are shown with an arrow. Other bands above the monomer are different complexes of AP30 under the condition. FIG. 3B (right image) shows the native gel showing hTETII and AP30 protein profiles before and after conditioning (complex formation at 65° C. in the presence of cobalt acetate for 30 minutes, which yields mostly dodecameric complex). AP30 shows more uniform higher order complex formation.

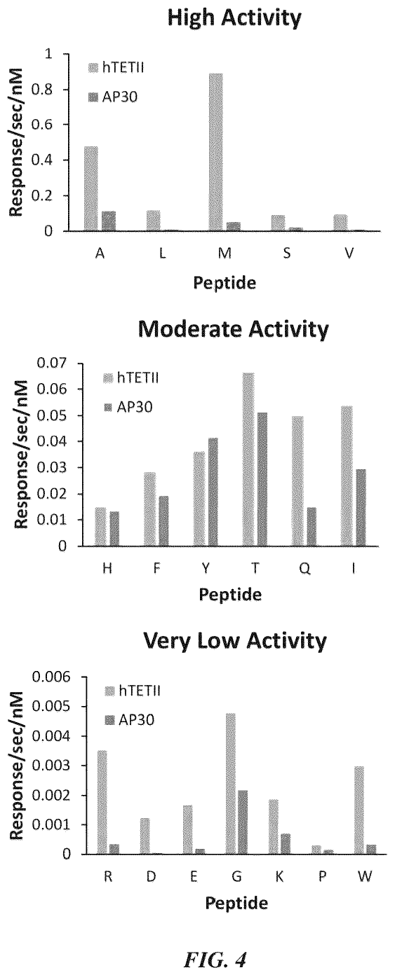

A real-time cleavage kinetics assay was performed using amino acid-AMC (7-Amino-4-methylcoumarin) substrates, with cleavage kinetics followed in real time at excitation wavelength of 357 nm and emission wavelength of 450 nm, at 30° C. to measure the increase in fluorescence at 441 nm. FIG. 4 shows three bar charts comparing the inherent cleavage rates and substrate specificities by hTETII and AP30 for 18 individual amino acids categorized as per the activity level (high activity: top chart; moderate activity: middle chart; very low activity: bottom chart). The inherent cleavage rates were derived for each residue at an optimal concentration of aminopeptidase which were determined by aminopeptidase titration. The rates were calculated by exponential fits of the intensity change of the time course. These results showed that AP30 has slower inherent cleavage rates than hTETII for individual amino acids.

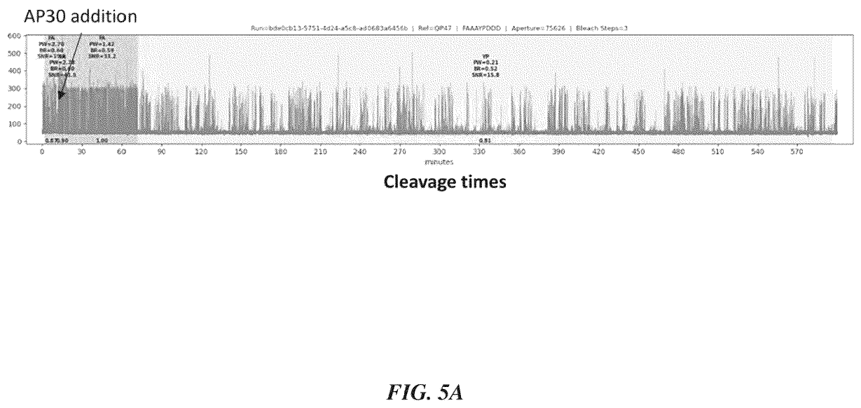

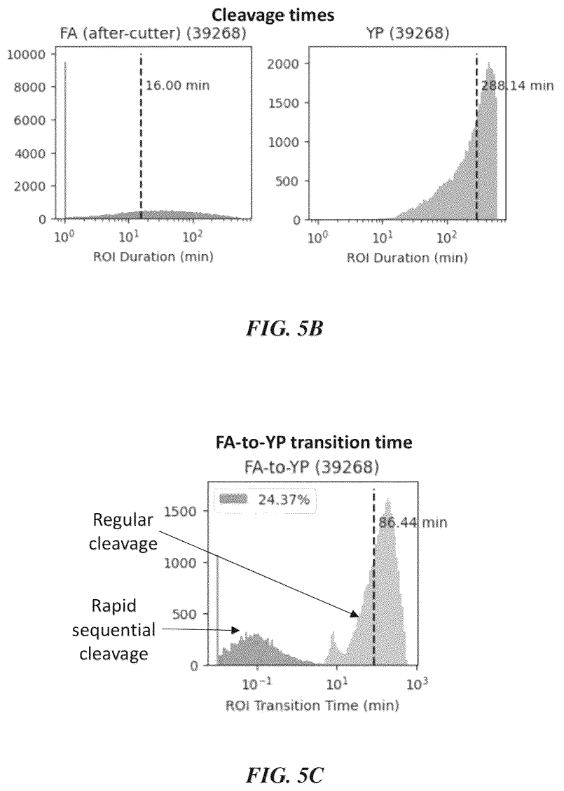

A protein sequencing assay was performed with AP30 cutter (5 μM), PS610 (50 nM) recognizer and QP47 peptide (FAAAYPDDD) (SEQ ID NO: 46). FIG. 5A shows a representative trace showing the first RS and the last RS recognition by PS610 as the cleavage progressed. FIG. 5B shows plots showing the mean cleavage times for FA (left plot) and YP (right plot) RSs. FIG. 5C shows a plot showing the rapid sequential cleavage population and regular cleavage population. The RSC population with AP30 was only ˜25%, which was lower compared to hTETII. These results showed that AP30 shows a smaller population of rapid sequential cleavage (RSC) than hTETII.

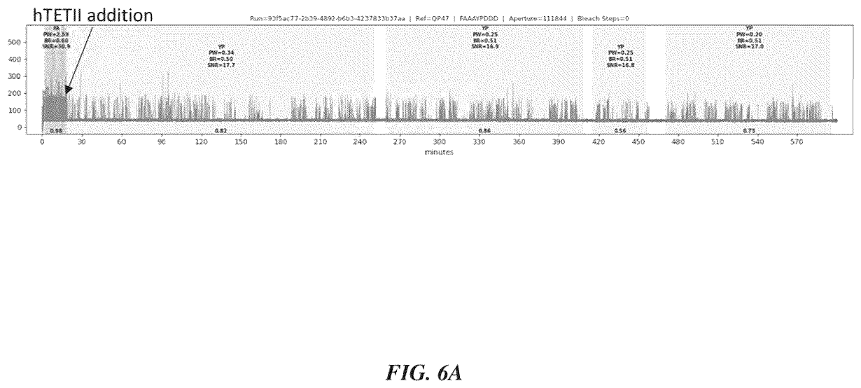

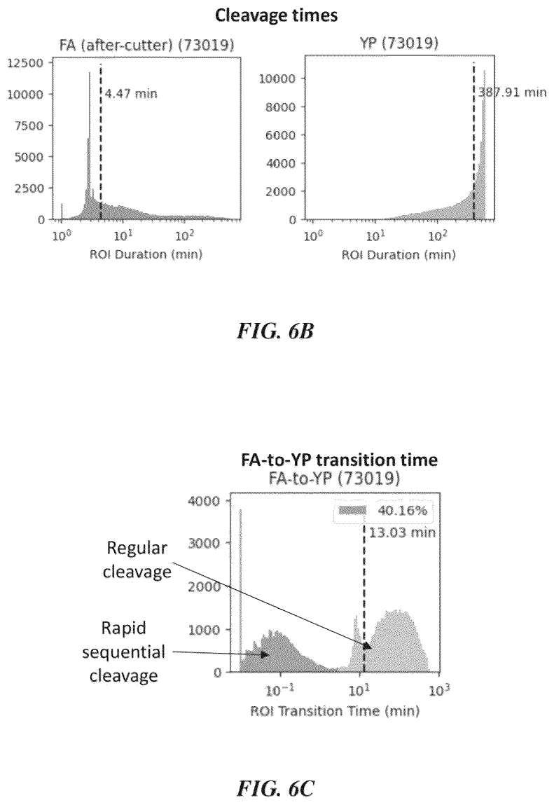

A protein sequencing assay was performed with 1 μM hTETII cutter, PS610 (50 nM) recognizer and QP47 peptide (FAAAYPDDD) (SEQ ID NO: 46). FIG. 6A shows a representative trace showing the first and the last RSs by PS610 as the cleavage progressed. FIG. 6B shows plots showing the mean cleavage times for FA and YP RSs. FIG. 6C shows a plot showing the rapid sequential cleavage population and regular cleavage population. RSC population is ˜40%. These results showed that hTETII has a larger population of rapid repetitive cleavage (RSC) than AP30.

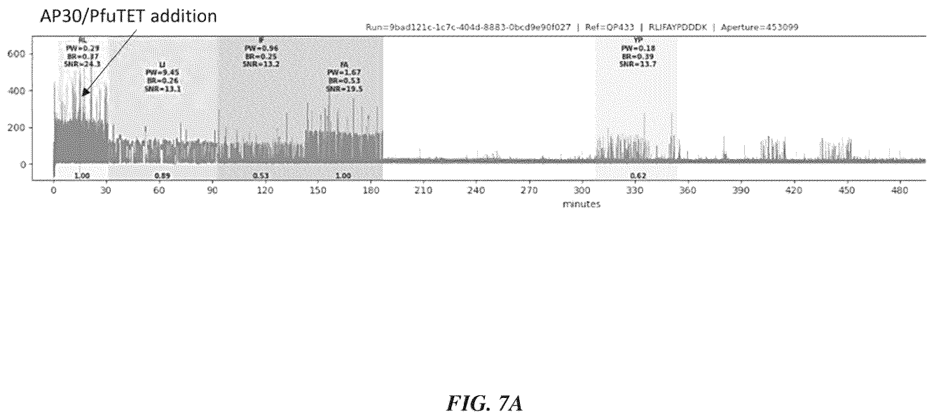

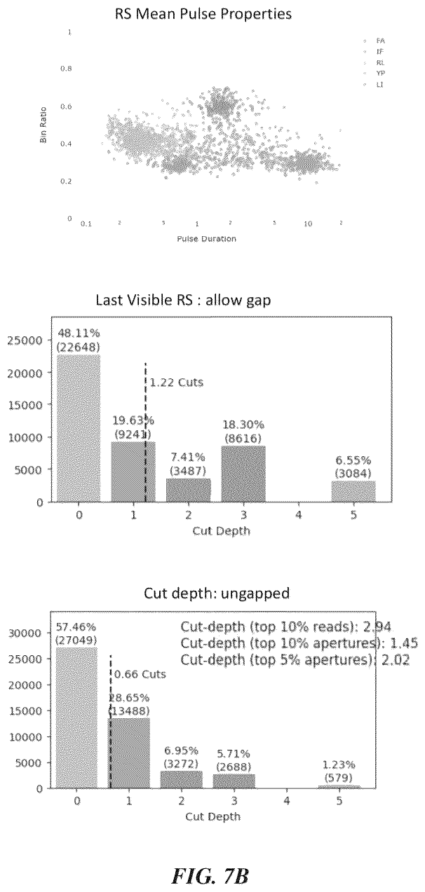

A protein sequencing assay was performed with AP30 (1 μM)/PfuTET (40 μM), an alternate cutter combination, along with PS610 (50 nM), PS557 (250 nM) and PS621 (250 nM) recognizers and QP433 peptide (RLIFAYPDDD) (SEQ ID NO: 47). FIG. 7A shows a representative trace showing the five RSs identified by the recognizers as the cleavage progressed. FIG. 7B shows a Bin Ratio vs. Pulse duration plot with separated clusters for recognized RSs (top plot), and plots showing the cut depths and proportion of each RS recognized in the reads with (middle plot) and without (bottom plot) gap (no missed recognizable residue).

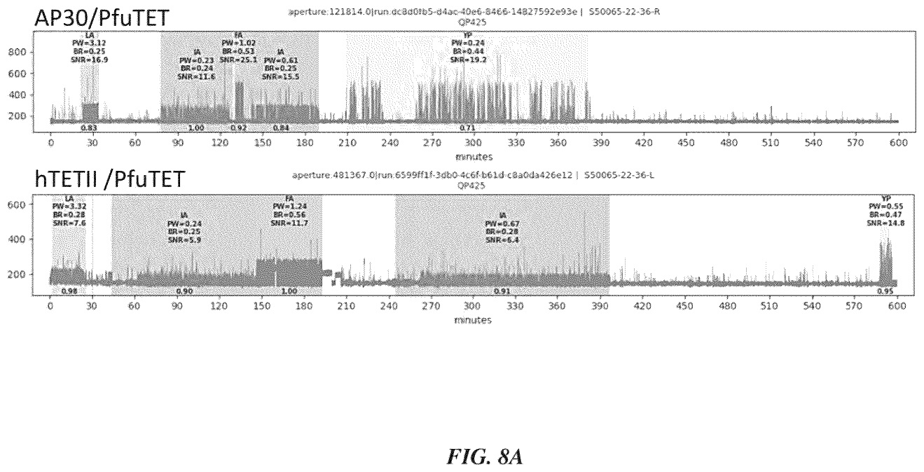

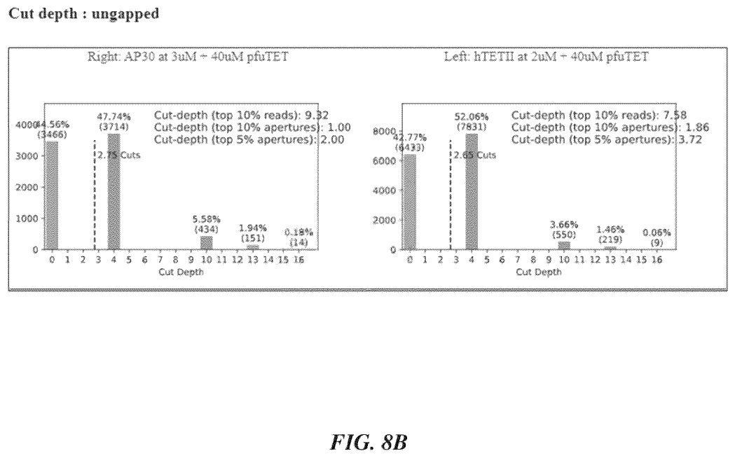

A protein sequencing assay was performed with AP30 (3 μM) or hTETII (2 μM)/PfuTET (40 μM) aminopeptidase combination, along with PS610 (50 nM) and PS557 (250 nM) recognizers and QP425 (LASSIAEANRFADIADYP) (SEQ ID NO: 48) on the same chip. FIG. 8A shows representative traces for reactions with AP30 and hTETII showing RSs identified by the recognizers as the cleavage progressed. FIG. 8B shows plots showing cut depths and proportions of each RS recognized in the reads without gap (no missed recognizable residue).

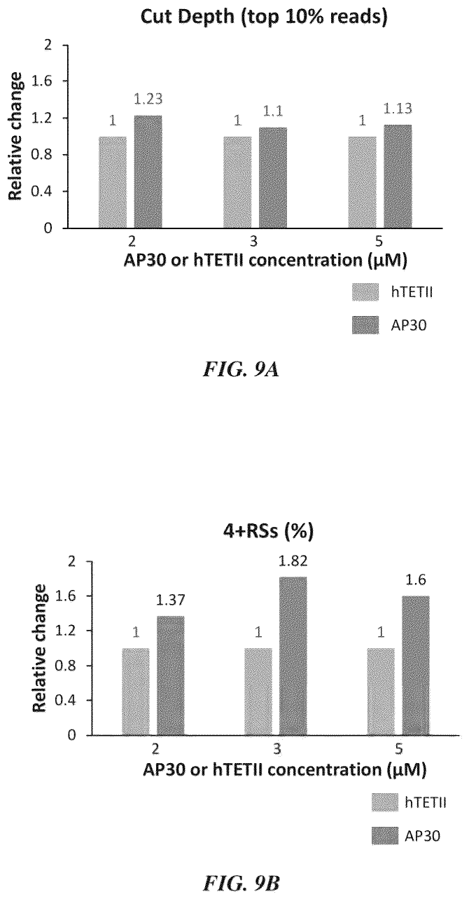

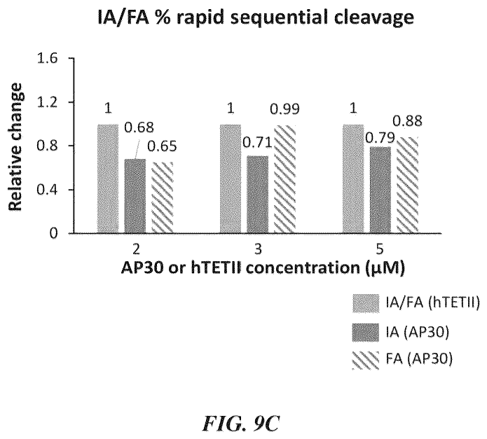

FIGS. 9A-9C illustrate that AP30 improved cutting performance by increasing cleavage activity and reducing rapid sequential cleavage. The bar charts in FIGS. 9A-9C show the improvements of cut depth (FIG. 9A) and % of 4+ RSs reads (FIG. 9B) at different AP30 concentrations with 40 μM pfuTET, PS610 (50 nM) and PS557 (250 nM) recognizers on QP425 (LASSIAEANRFADIADYP) (SEQ ID NO: 48). The change was calculated relative to hTETII tested at the same concentration on the same chip. AP30 reduces rapid sequential cleavage of IA and FA RSs (FIG. 9C). The Cleavage activity was also improved in the chip readout because the number of useful reads (4+RSs) increased with AP30/PfuTET combination.

Tables 3 and 4 show example results from these studies showing that AP30 improved cutting performance (cut depth and % of useful reads) by increasing cleavage activity and reducing rapid sequential cleavage.

TABLE 3
Results from studies evaluating cutting performance

| | | | | cut | | | | | |
|---|---|---|---|---|---|---|---|---|---|
| | | | | depth | cut | LA- | LA-IA- | | |
| | | | | (10% | depth | IA- | FA | | 4 + RS |
| Peptide | exp/ctrl | hTETII/AP30 | PfuTET | reads) | (% change) | FA | (% change) | 4 + RS | (% change) |
| QP425 | ctrl | hTETII | 40 μM | 7.58 | | 3.52 | | 1.93 | |
| | | (2 μM) | | | | | | | |
| QP425 | exp | AP30 | 40 μM | 9.32 | 23% | 5.36 | 52% | 2.65 | 37% |
| | | (2 μM) | | | | | | | |
| QP425 | ctrl | hTETII | 40 μM | 4.7 | | 0.81 | | 0.28 | |
| | | (3 μM) | | | | | | | |
| QP425 | exp | AP30 | 40 μM | 5.17 | 10% | 1.42 | 75% | 0.51 | 82% |
| | | (3 μM) | | | | | | | |
| QP425 | ctrl | hTETII | 40 μM | 5.32 | | 1.72 | | 0.5 | |
| | | (5 μM) | | | | | | | |
| QP425 | exp | AP30 | 40 μM | 6 | 13% | 2.6 | 49% | 0.8 | 60% |
| | | (5 μM) | | | | | | | |

TABLE 4
Results from studies evaluating cutting performance (continued from Table 3)

| | | | | | cleavage | | IA RS | | FA RS |
|---|---|---|---|---|---|---|---|---|---|
| | | | | cleavage | activity | IA RS | missing | FA RS | missing |
| Peptide | exp/ctrl | hTETII/AP30 | PfuTET | activity | (% increase) | missing | (% change) | missing | (% change) |
| QP425 | ctrl | hTETII | 40 μM | 4.68 | | 11.97 | | 4.47 | |
| | | (2 μM) | | | | | | | |
| QP425 | exp | AP30 | 40 μM | 8.86 | 89% | 8.19 | −32% | 2.93 | −35% |
| | | (2 μM) | | | | | | | |
| QP425 | ctrl | hTETII | 40 μM | 3.42 | | 26.72 | | 2.46 | |
| | | (3 μM) | | | | | | | |
| QP425 | exp | AP30 | 40 μM | 6.22 | 82% | 18.88 | −29% | 2.44 | −1% |
| | | (3 μM) | | | | | | | |
| QP425 | ctrl | hTETII | 40 μM | 4.21 | | 18.7 | | 2.72 | |
| | | (5 μM) | | | | | | | |
| QP425 | exp | AP30 | 40 μM | 8.14 | 93% | 14.84 | −21% | 2.39 | −12% |
| | | (5 μM) | | | | | | | |

### Example 2. Evaluation of Pyrococcus horikoshii TET III Aminopeptidases

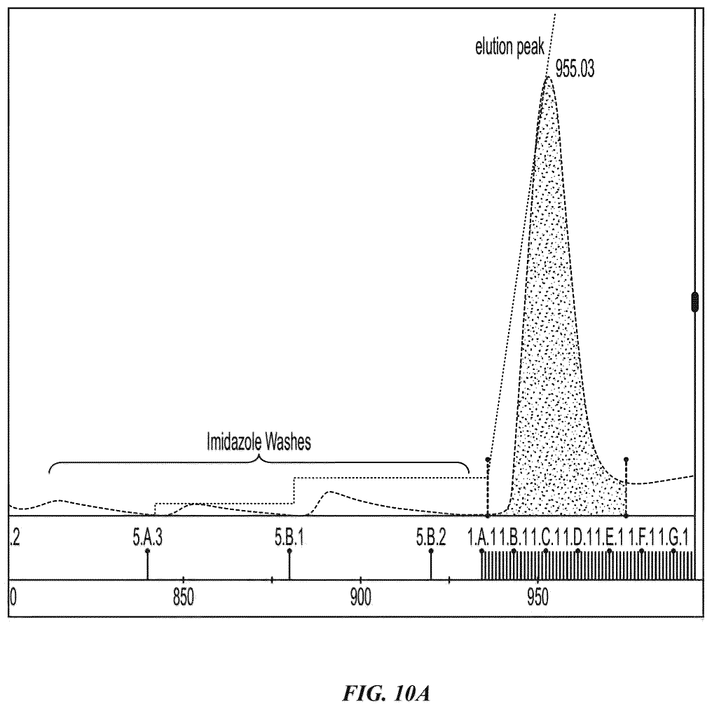

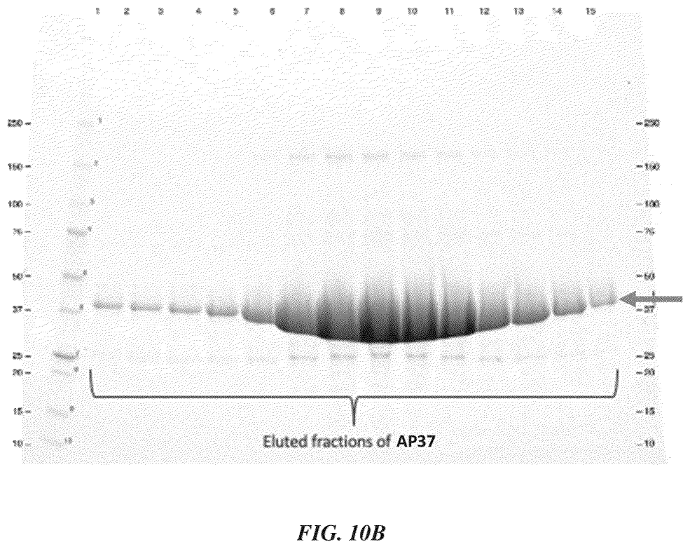

In this example, cutting performance was evaluated for AP37, a cleaving reagent comprising Pyrococcus horikoshii TET III Aminopeptidase (hTET III) and a C-terminal tag. hTET III is a homolog of PfuTET, which was used in these studies for comparative purposes relative to AP37. AP37 was expressed in E. coli and the cell free extract was resolved in talon affinity column in AKTA. FIG. 10A shows an example chromatogram showing AP37 enrichment and the eluted peak.

FIG. 10B shows talon affinity column purification fractions resolved on SDS PAGE gel to show AP37 separation. The eluted peak fractions with monomer (40.33 kDa) are shown with an arrow. Other bands above the monomer are different complexes of AP37 under the condition. Results obtained from HPLC assay data showed improvements in cleavage activity of AP37 after cobalt acetate and heat conditioning. Table 5 displays the activity data at 1 μM and 10 μM aminopeptidase concentration (before and after conditioning) on peptides with distinct N-terminal amino acid.

TABLE 5
Results from HPLC assay data
Percent N-Terminal Residue Cut

| | AP37 before | | AP37 after | |
|---|---|---|---|---|
| | conditioning | | conditioning | |
| Peptide | 1 μM | 10 μM | 1 μM | 10 μM |
| WKELDEESILKQK | 18 | 55 | 97 | 99 |
| IAKLDEESILKQK | 13 | 57 | 74 | 100 |
| RRLDEESILKQK | 8 | 26 | 70 | 100 |
| KKLDEESILKQK | 11 | 71 | 61 | 100 |

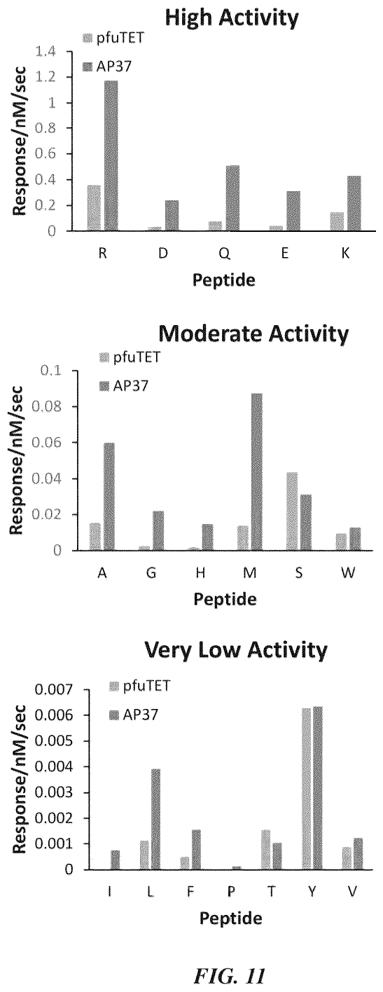

A real-time cleavage kinetics assay was performed using amino acid-AMC (7-Amino-4-methylcoumarin) substrates, with cleavage kinetics followed in real time at excitation wavelength of 357 nm and emission wavelength of 450 nm, at 30° C. to measure the increase in fluorescence at 441 nm. FIG. 11 shows three bar charts comparing the inherent cleavage rates and substrate specificities by PfuTET and AP37 for 18 individual amino acids categorized as per the activity level (high activity: top chart; moderate activity: middle chart; very low activity: bottom chart). The inherent cleavage rates were derived for each residue at an optimal concentration of aminopeptidase which were determined by aminopeptidase titration. The rates were calculated by exponential fits of the intensity change of the time course. These results showed that AP37 has faster inherent cleavage rates than PfuTET for individual amino acids.

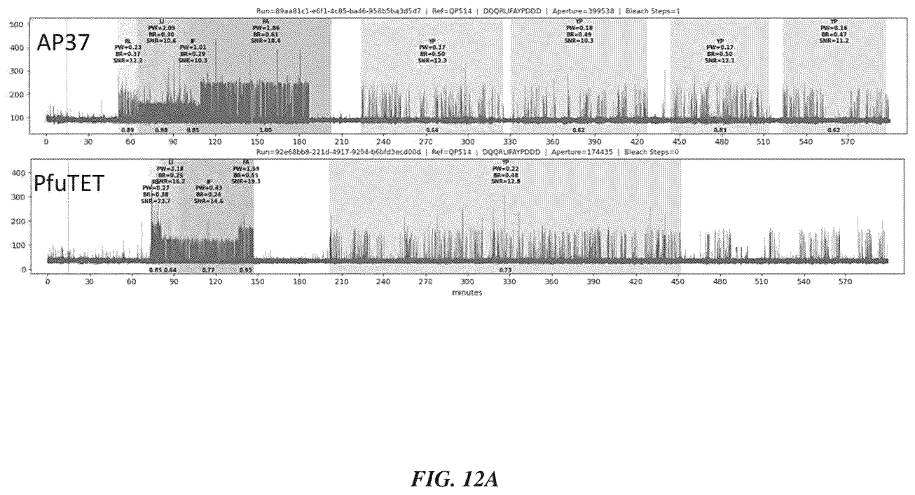

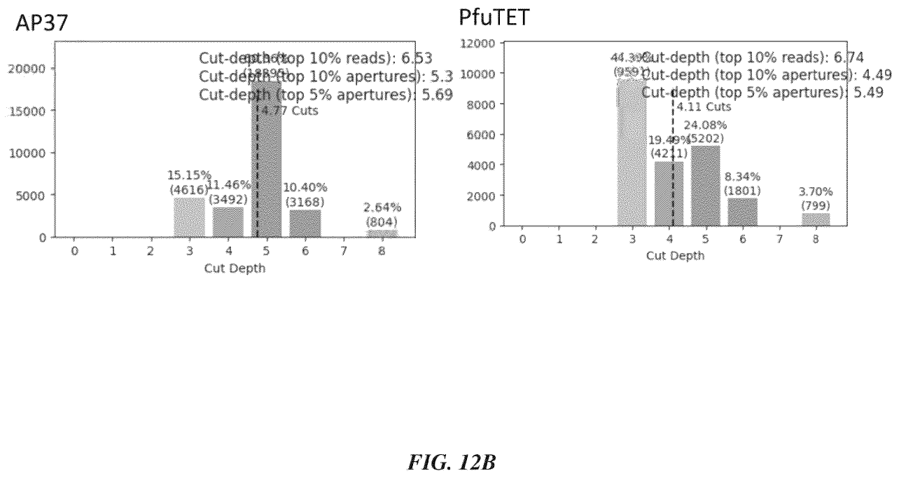

A protein sequencing assay was performed with AP37 (40 μM) or PfuTET (40 μM) aminopeptidase, along with PS610 (50 nM), PS961 (250 nM), PS961 (250 nM) recognizers and QP514 (DQQRLIFAYPDDD) (SEQ ID NO: 49) on separate chips. FIG. 12A shows representative traces for reaction with AP37 or PfuTET showing RSs identified by the recognizers as the cleavage progressed. FIG. 12B shows plots showing cut depths and proportions of each RS recognized in the reads without gap (no missed recognizable residue). These results showed that AP37 shows better efficiency cleaving arginine than PfuTET when alone.

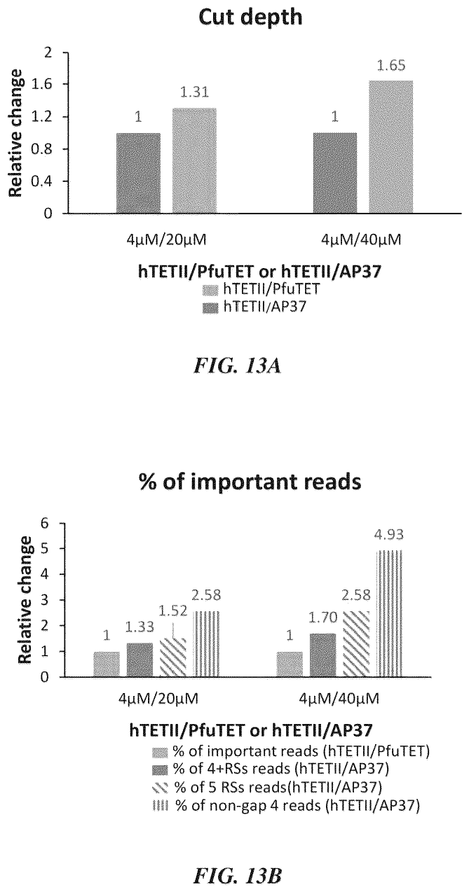

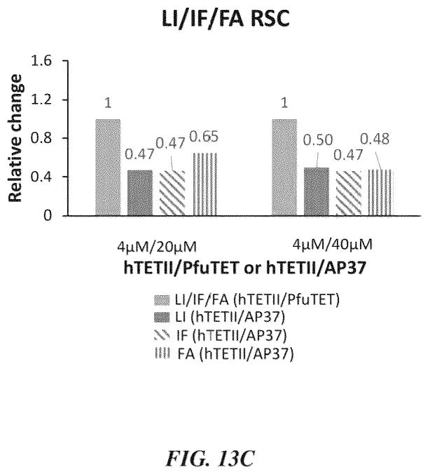

A protein sequencing assay was performed at two different AP37 concentrations (20 μM/40 μM), 4 μM hTETII, and PS610 (50 nM), PS557 (250 nM) and PS691 (100 nM) recognizers on QP433 (RLIFAYPDDD) (SEQ ID NO: 47). FIGS. 13A-13C show plots depicting the results from these studies, with changes calculated relative to PfuTET tested at the same concentration on the same chip. hTETII was kept at 4 μM in all conditions. FIG. 13A shows the improvements in cut depth. FIG. 13B shows improvements in % of important reads. FIG. 13C shows that AP37 reduced rapid sequential cleavage (RSC) of LI, IF, and FA RSs.

### Example 3. Evaluation of AP30 and AP37 Aminopeptidase Combination

In this example, protein sequencing assays were performed to evaluate the combination of AP30 and AP37 in a sequencing reaction.

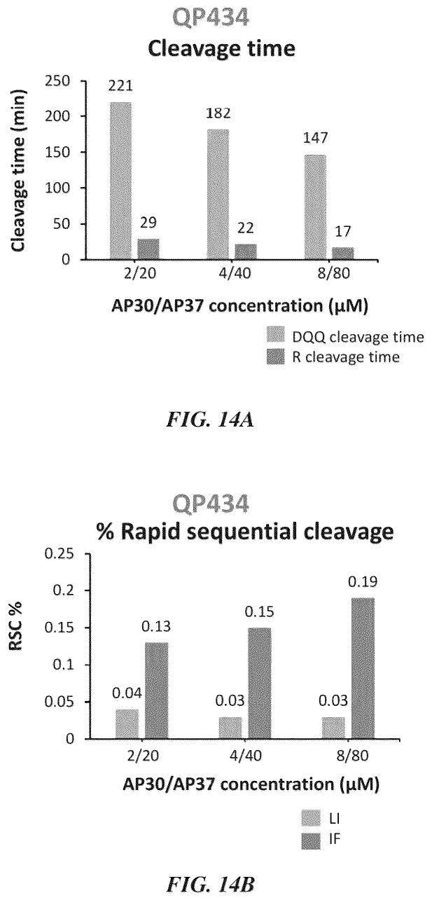

FIGS. 14A-14B show bar charts illustrating the effect of different AP30/AP37 proportions with PS610 (50 nM), PS691 (100 nM), and PS961 (250 nM) recognizers, on time to cleave DQQ motif and R residue (FIG. 14A) and RS rapid sequential cleavage (FIG. 14B). Increasing aminopeptidases concentration during sequencing of QP434 (DQQRLIFAG) showed an increase in cleavage rate for both dark amino acids (DQQ motif) and for the visible amino acid R, which led to an increase in the rapid sequential cleavage of IF.

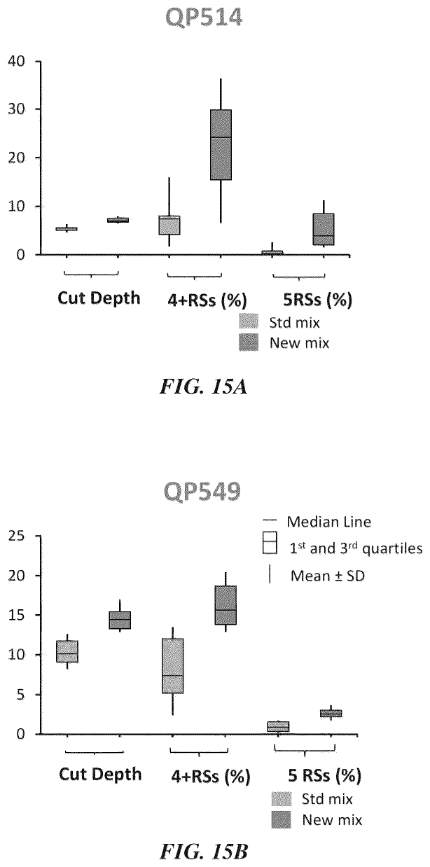

FIGS. 15A-15B show bar charts showing the distribution of the performance metrics (cut depth, % of 4+RSs reads, and % of 5 RSs reads) with the recognizers mix of PS610 (50 nM), PS961 (250 nM), and PS691 (100 nM), and AP30/AP37 at 4 μM/40 μM on QP514 (DQQRLIFAYPDDD) (SEQ ID NO: 49) (FIG. 15A, seven technical replicates) and at 10 μM/60 μM concentration on QP549 (DQQIASSRLAASFAAQQYPDDD1) (SEQ ID NO: 50) (FIG. 15B, six technical replicates). Standard recognizers and aminopeptidase conditions were used as a control on the same chip [PS610 (50 nM), PS557 (250 nM) PS621 (250 nM) and hTETII/PfuTET at 1 μM/40 μM for QP514 and at 3 μM/60 μM for QP549].

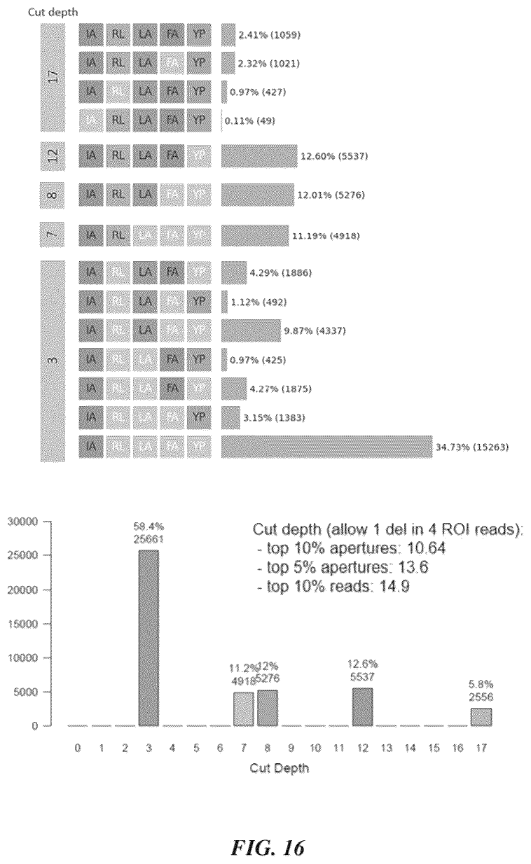

FIG. 16 shows reads breakdown and abundance of ungapped reads, with single deletion allowed in reads of length of 4, obtained from one of the chip runs by the recognizers and aminopeptidases mix of PS961/PS691 at 250/100 nM and AP30/hTETIII at 10/60 μM, on QP549. The updated combination of aminopeptidases and recognizers, on QP549 achieved unprecedented cut depth values of >9 cut depth for the top 10% of apertures in multiple runs. This run reached 10/10, with cut depth of 10.64 for the top % of apertures with single rapid sequential cleavage allowed in reads of length of 4.

### Example 4. Evaluation of Yersinia pestis Xaa-Prolyl aminopeptidase (yPIP)

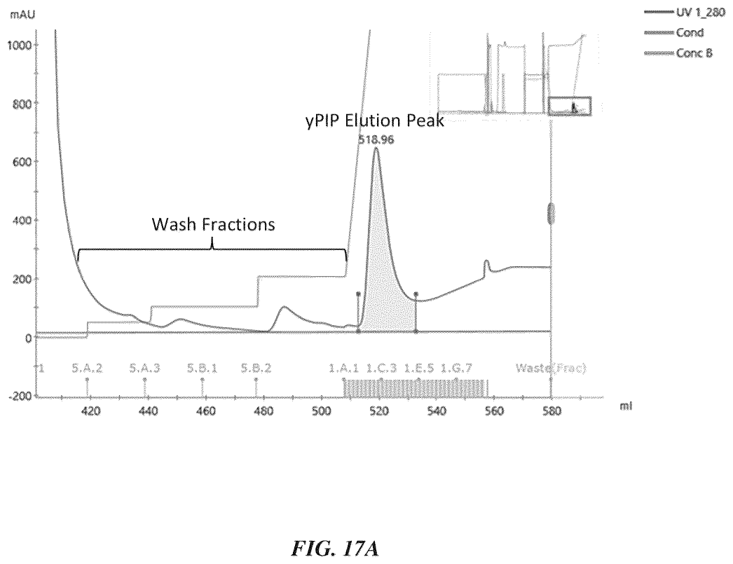

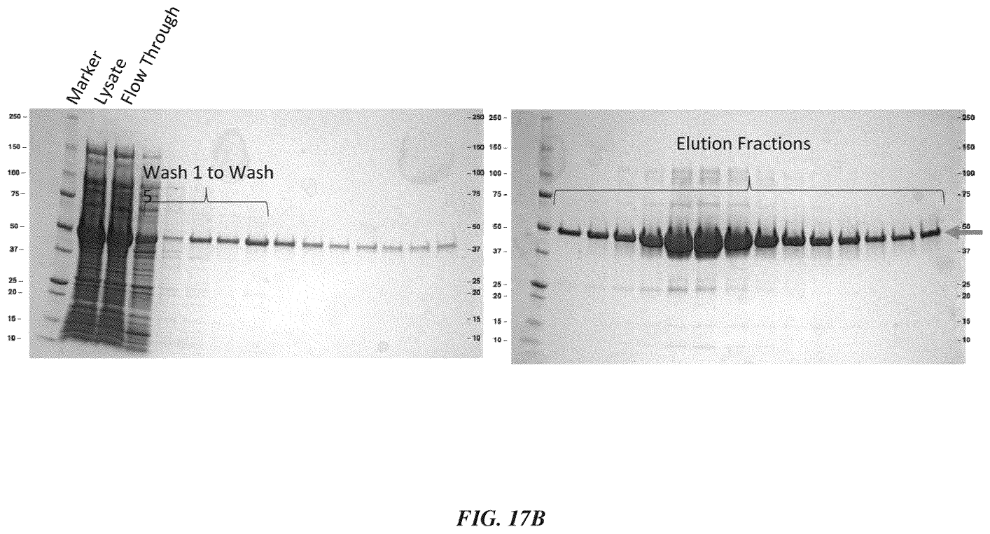

Xaa-Prolyl aminopeptidase (yPIP) from Yersinia pestis, also known as proline aminopeptidase PII, is a monomeric AP that has specific activity towards N-terminal X-P motifs. In this example, cutting performance was evaluated for a cleaving reagent comprising yPIP and a C-terminal 6×His-tag. yPIP (BC-B1 batch) was expressed in E. coli and the cell free extract was prepared after the cell lysis and resolved in talon affinity column in AKTA. FIG. 17A shows an example chromatogram showing yPIP separation and the eluted peak. FIG. 17B shows Talon affinity column purification fractions resolved on SDS PAGE gel to show yPIP enrichment. The eluted peak fractions at 51 kDa are shown with an arrow.

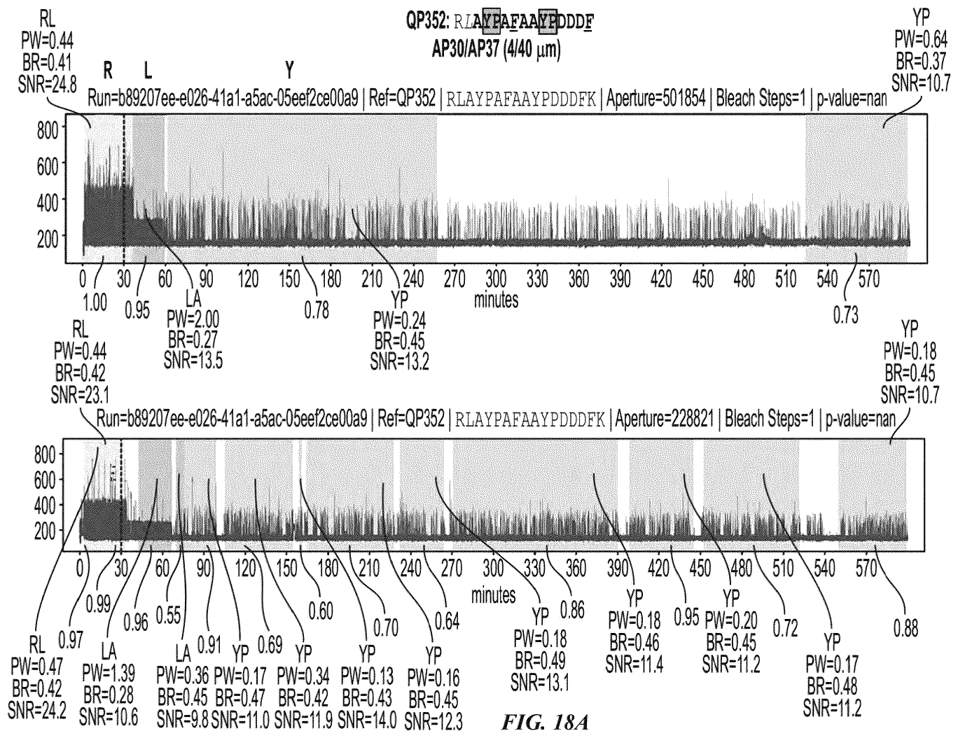

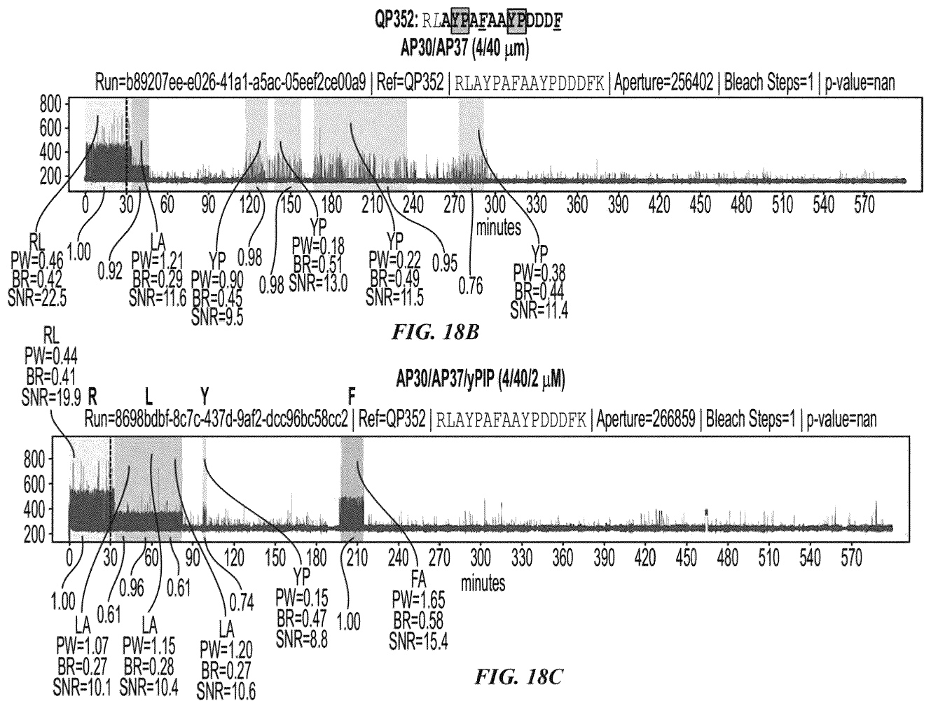

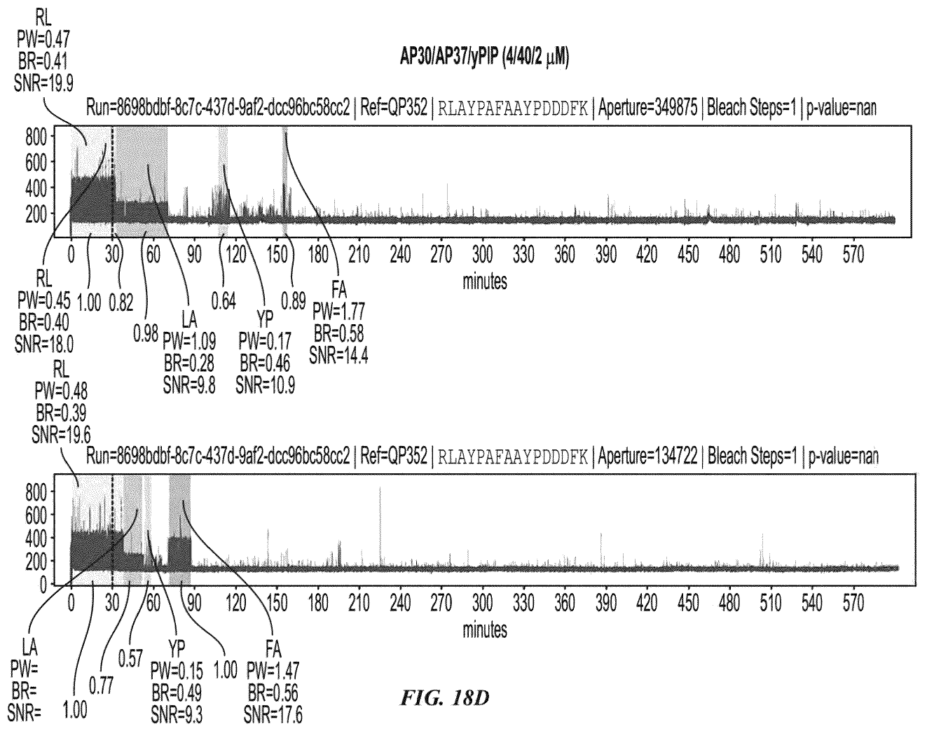

FIGS. 18A-18D show representative traces showing the effect of adding yPIP (2 μm) in the AP30/AP37 (4/40 μM) aminopeptidase combination in a protein sequencing assay. The protein sequencing assay was carried out with (FIGS. 18A-18B) AP30 (1 μM)/AP37 (40 μM), and (FIG. 18C-18D) AP30 (1 μM)/AP37 (40 μM)/yPIP (2 μM) combinations on QP352 (RLAYPAFAAYPDDDF) (SEQ ID NO: 51) peptide along with PS610 (50 nM)/PS961 (125 nM)/PS1122 (250 nM) recognizers. These results showed that yPIP in the AP30/AP37 aminopeptidase combination helps cleaves past the YP motif more efficiently.

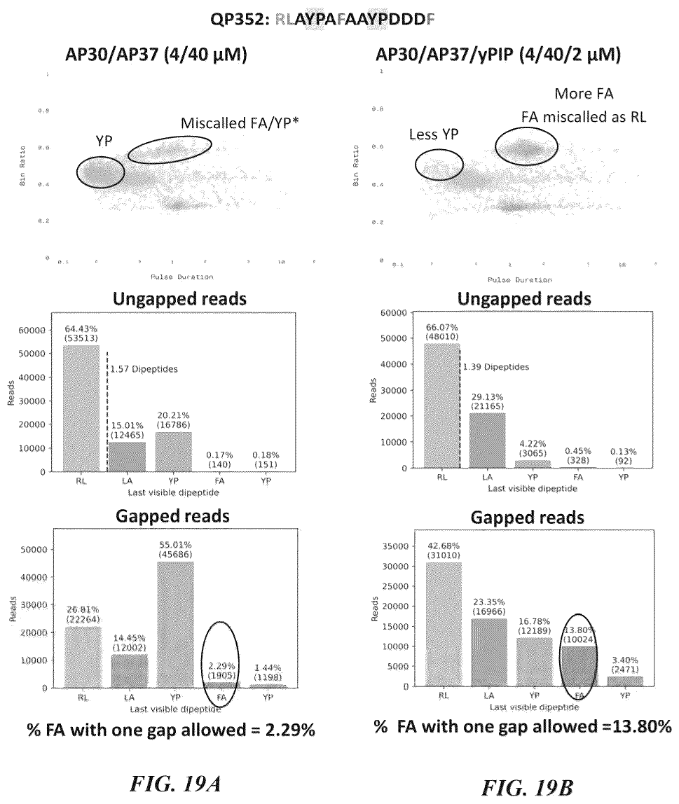

FIGS. 19A-19B show example results from protein sequencing assays with (FIG. 19A) AP30 (4 μM)/AP37 (40 μM), and (FIG. 19B) AP30 (4 μM)/AP37 (40 μM)/yPIP (2 μM) combinations on QP352 (RLAYPAFAAYPDDDF) (SEQ ID NO: 51) peptide along with PS610 (50 nM)/PS961 (125 nM)/PS1122 (250 nM) recognizers. Bin Ratio vs. Pulse duration plot with separated clusters for recognized RSs are shown in top panels. Bar plots are shown for the cut depths and proportion of each RS recognized in the reads without gap (middle panels) and with gaps (bottom panels). These results showed that yPIP in the AP30/AP37 aminopeptidase combination cleaves past the YP motif reaching FA more efficiently.

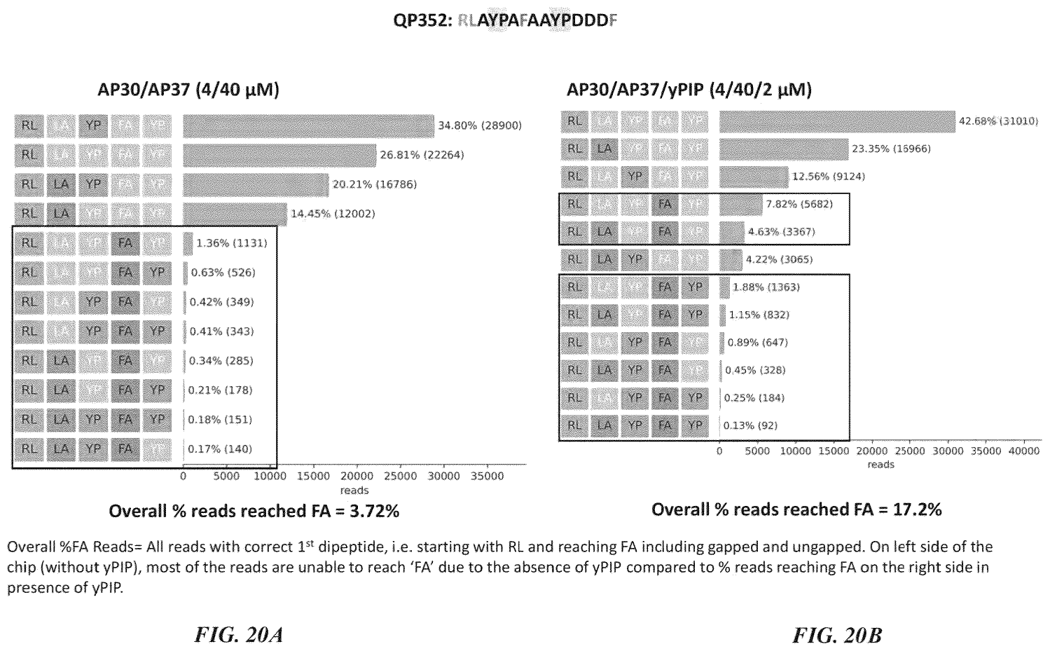

FIGS. 20A-20B show reads proportion breakdown for the QP352 (RLAYPAFAAYPDDDF) (SEQ ID NO: 51) protein sequencing assay with (FIG. 20A) AP30 (4 μM)/AP37 (40 μM), and (FIG. 20B) AP30 (4 μM)/AP37 (40 μM)/yPIP (2 μM) combinations along with PS610 (50 nM)/PS961 (125 nM)/PS1122 (250 nM) recognizers. These results further demonstrated that yPIP in the AP30/AP37 aminopeptidase combination cuts past the YP motif reaching FA more efficiently.

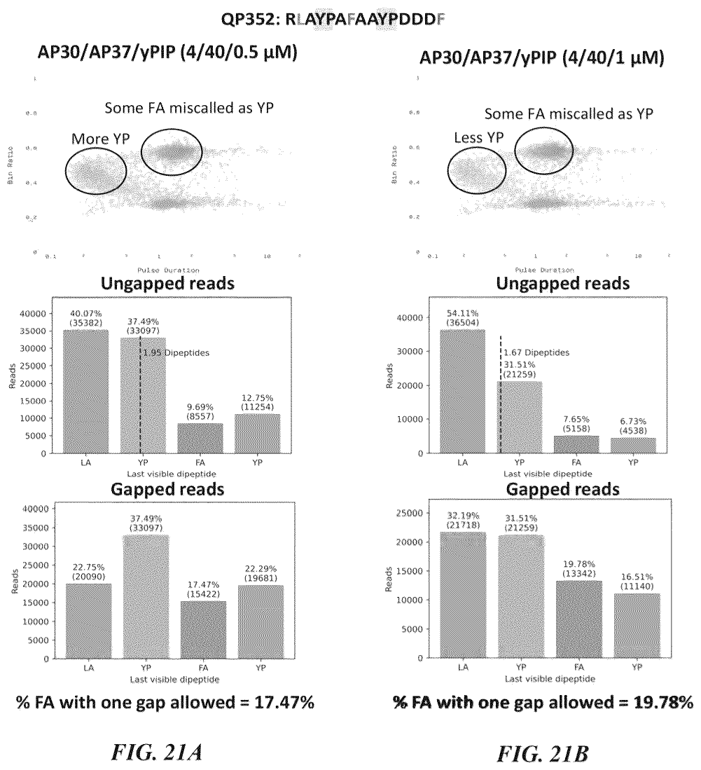

FIGS. 21A-21B show example results from protein sequencing assays with (FIG. 21A) AP30 (4 μM)/AP37 (40 μM)/yPIP (0.5 μM), and (FIG. 21B) AP30 (4 μM)/AP37 (40 μM)/yPIP (1 μM) combinations on QP352 (RLAYPAFAAYPDDDF) (SEQ ID NO: 51) peptide along with PS610 (50 nM)/PS961 (125 nM) recognizers. Bin Ratio vs. Pulse duration plot with separated clusters for recognized RSs are shown in top panels. The actual % FA should be higher for both yPIP concentrations, as FA is miscalled as YP. However, the density of FA miscalling is much higher for yPIP at 1 μM compared to FA miscalling for yPIP at 0.5 μM. Additionally, the YP cluster was denser for yPIP at 0.5 μM compared to the YP cluster for yPIP 1 μM. Taken together, these observations indicated that overall performance of yPIP at 1 μM was more favorable than yPIP at 0.5 μM under these reaction conditions. Bar plots are shown in FIG. 21 for the cut depths and proportion of each RS recognized in the reads without gap (middle panels) and with gaps (bottom panels). A higher 1^{st} YP and lower FA was indicative of inadequate cleavage (the higher the YP compared to FA; less YP cleavage); a lower 1^{st} YP and higher FA was indicative of YP cleavage and YP deletions (the lower the YP compared to FA; higher the RSC); and a similar YP and FA was considered ideal (good YP cleavage and less RSC).

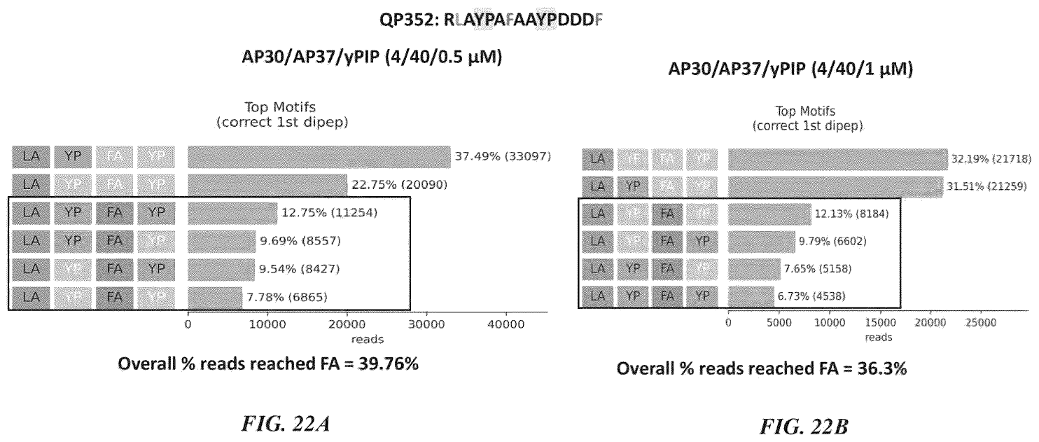

FIGS. 22A-22B show reads proportion breakdown for the QP352 (RLAYPAFAAYPDDDF) (SEQ ID NO: 51) protein sequencing assay with (FIG. 22A) AP30 (4 μM)/AP37 (40 μM)/yPIP (0.5 μM), and (FIG. 22B) AP30 (4 μM)/AP37 (40 μM)/yPIP (1 μM) combinations along with PS610 (50 nM)/PS961 (125 nM) recognizers combinations along with PS610 (50 nM)/PS961 (125 nM) recognizers. These results indicated that yPIP at 1 μM shows better performance than 0.5 μM in AP30/AP37 combination under these reaction conditions.

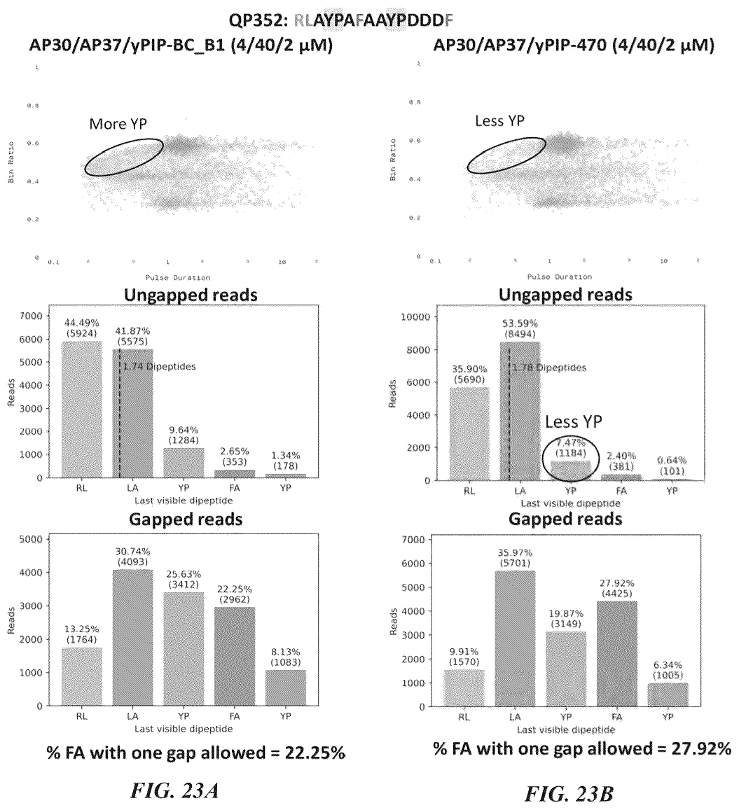

FIGS. 23A-23B show example results from protein sequencing assays with (FIG. 23A) AP30 (4 μM)/AP37 (40 μM))/yPIP-BC_B1 (2 μM), and (FIG. 23B) AP30 (4 μM)/AP37 (40 μM)/yPIP-470 (2 μM) combinations on QP352 (RLAYPAFAAYPDDDF) (SEQ ID NO: 51) peptide along with PS610 (50 nM)/PS961 (125 nM)/PS1122 (250 nM) recognizers. Bin Ratio vs. Pulse duration plot with separated clusters for recognized RSs are shown in top panels. Bar plots are shown for the cut depths and proportion of each RS recognized in the reads without gap (middle panels) and with gaps (bottom panels). yPIP470 was stored at −20 C for >18 months and showed similar performance compared to freshly purified yPIP-BC_B1 in terms of % FA reads. However, yPIP470 showed slight increase in RSC of 1^{st} YP (i.e. lower YP followed by higher FA).

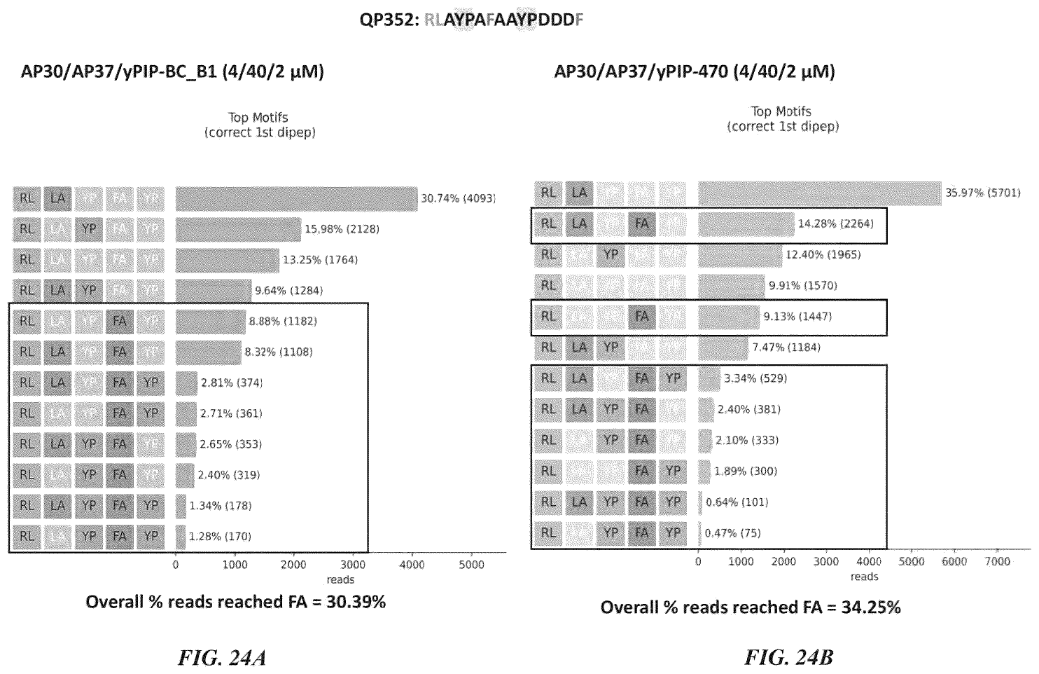

FIGS. 24A-24B show reads proportion breakdown for the QP352 (RLAYPAFAAYPDDDF) (SEQ ID NO: 51) protein sequencing assay with (FIG. 24A) AP30 (4 μM)/AP37 (40 μM))/yPIP-BC_B1 (2 μM), and (FIG. 24B) AP30 (4 μM)/AP37 (40 μM)/yPIP-470 (2 μM) combinations along with PS610 (50 nM)/PS961 (125 nM)/PS1122 (250 nM) recognizers.

### Example 5. Evaluation of AP70 Yersinia pestis Xaa-Prolyl aminopeptidase

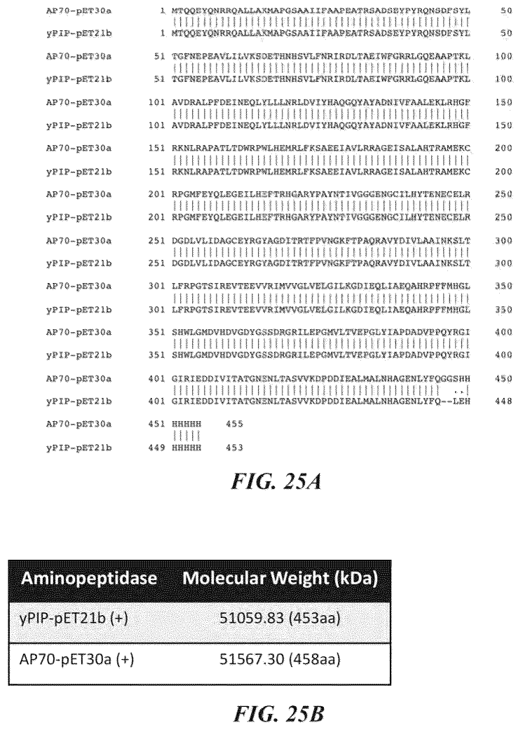

In this example, cutting performance was evaluated for AP70, which is a cleaving reagent comprising yPIP (truncated by two amino acids at the C-terminus) and a C-terminal GGS-6×His-tag. FIGS. 25A-25B show (FIG. 25A) yPIP (SEQ ID NO: 6) and AP70 (SEQ ID NO: 8) protein amino acid sequence alignment showing the C terminal 6×H tag and GGS-6×His-tag, respectively, and (FIG. 25B) expression constructs for yPIP and AP70 with molecular weights of the proteins.

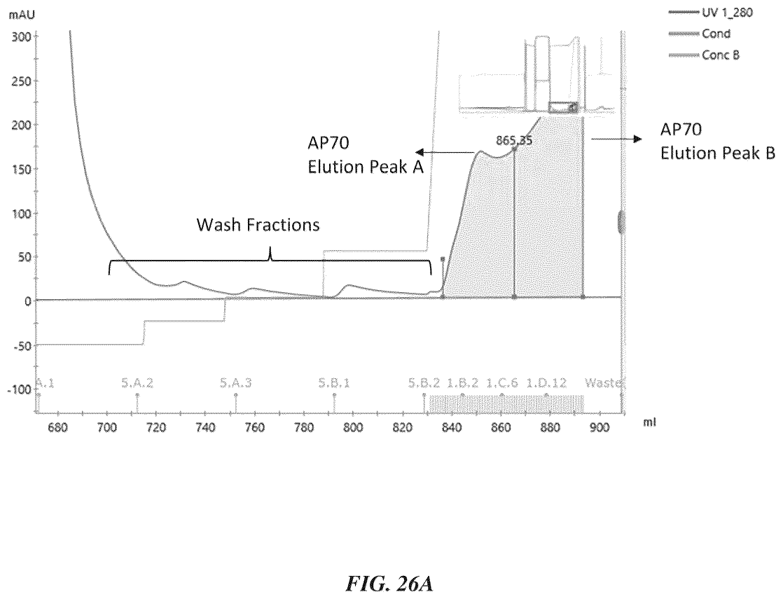

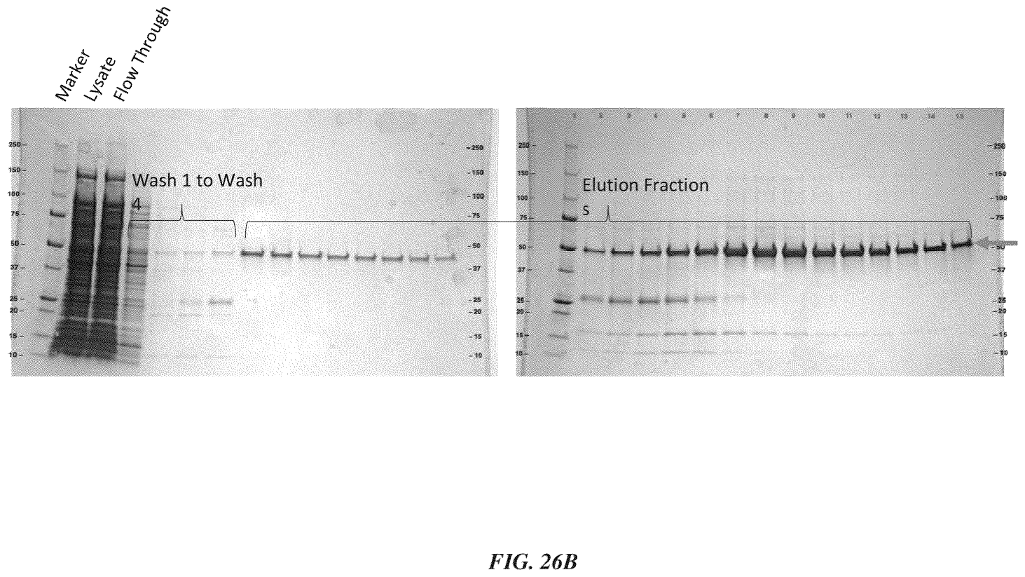

AP70 (BC-B1 batch) was expressed in E. coli and the cell free extract was prepared after the cell lysis and resolved in talon affinity column in AKTA. FIG. 26A shows an example chromatogram showing AP70 separation and the eluted peak. FIG. 26B shows talon affinity column purification fractions resolved on SDS PAGE gel to show AP70 enrichment. The eluted peak fractions at 51.56 kDa are shown with an arrow.

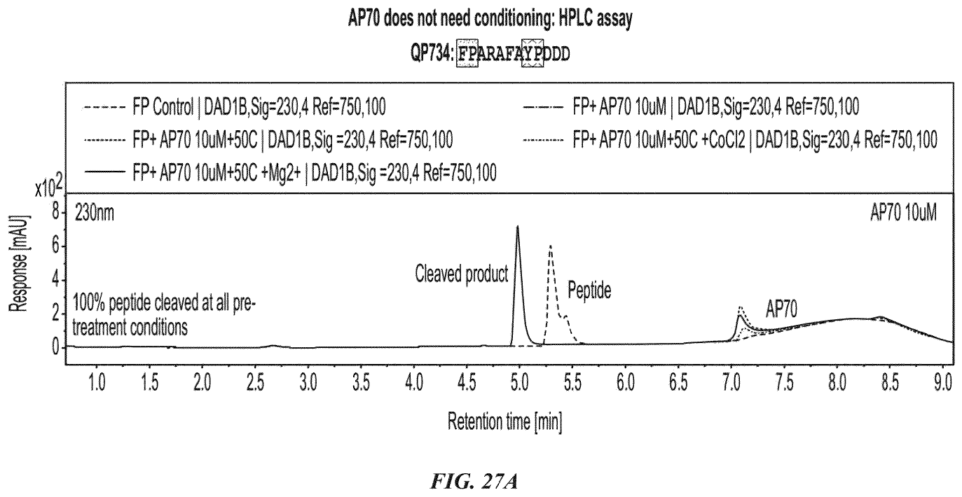

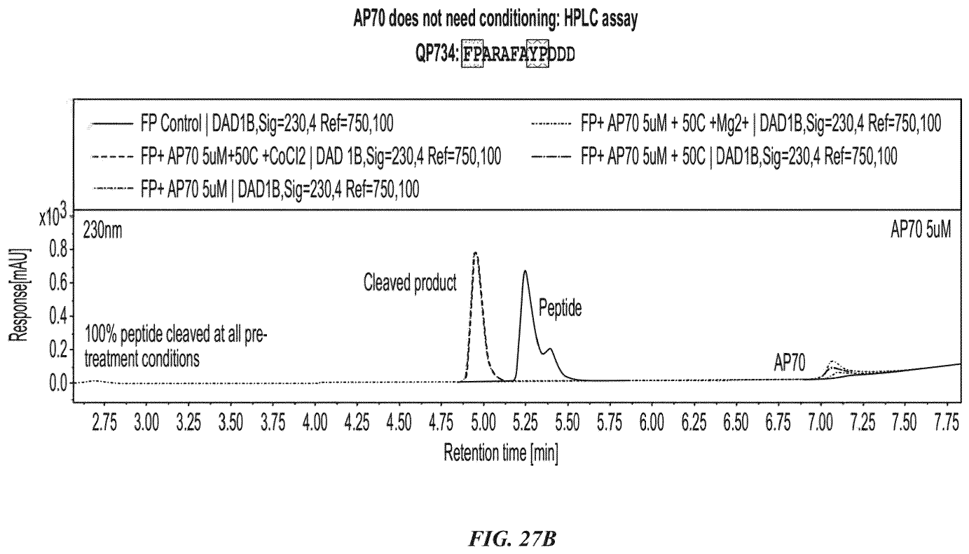

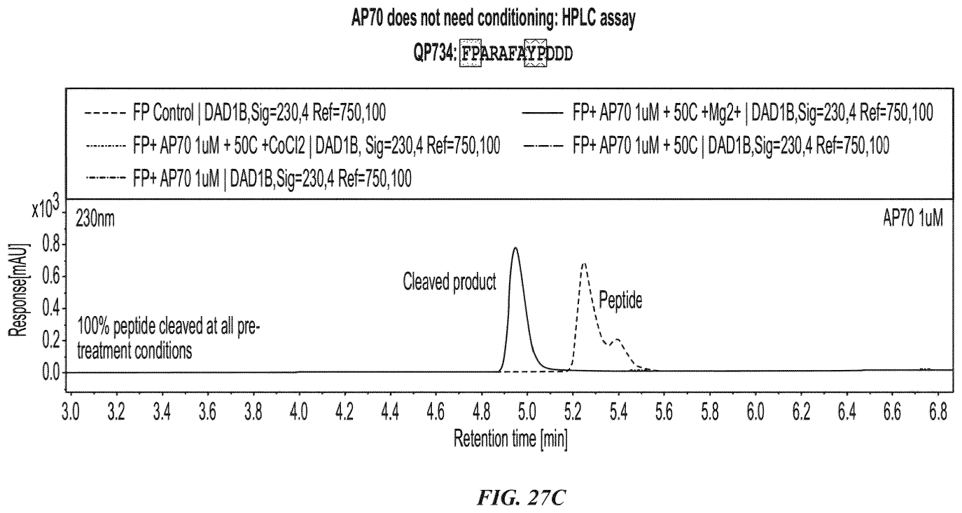

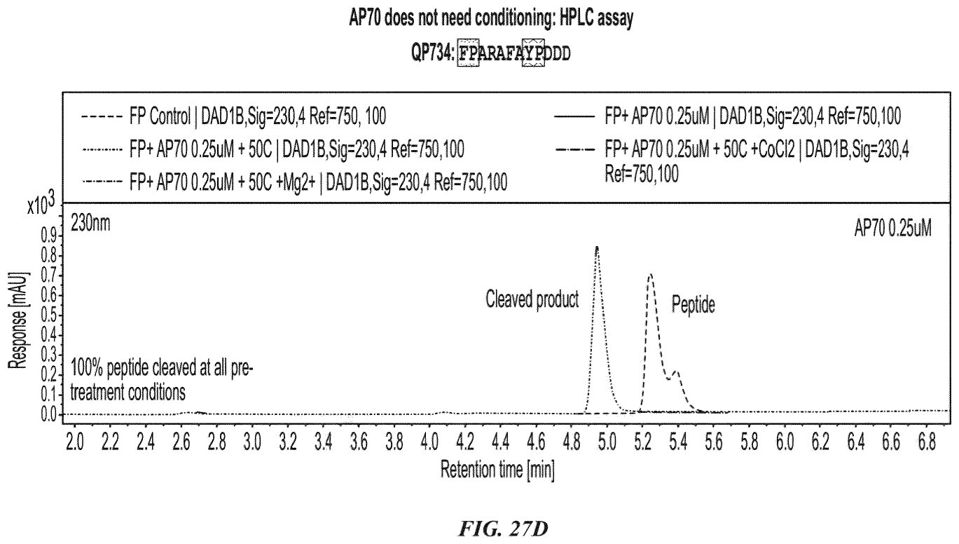

FIGS. 27A-27D show results from HPLC assay for QP734 (FPARAFAYPDDD) (SEQ ID NO: 52) peptide cleavage by AP70 at various concentrations after different conditioning treatments and unconditioned control. Reactions were resolved on a hydrophobic column in reverse phase HPLC. AP70 Pre-treatment Conditions: No treatment; 50° C., 30 min; 50° C.+Co²⁺5 mM, 30 min; 50° C.+Mg²⁺5 mM, 30 min. AP70 Pre-treatment Conditions: No treatment; 50° C., 30 min; 50° C.+Co²⁺5 mM, 30 min; 50° C.+Mg²⁺5 mM, 30 min.

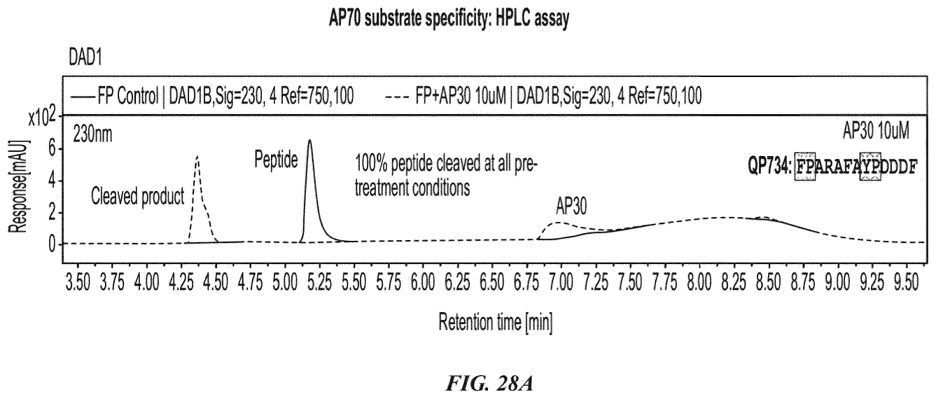

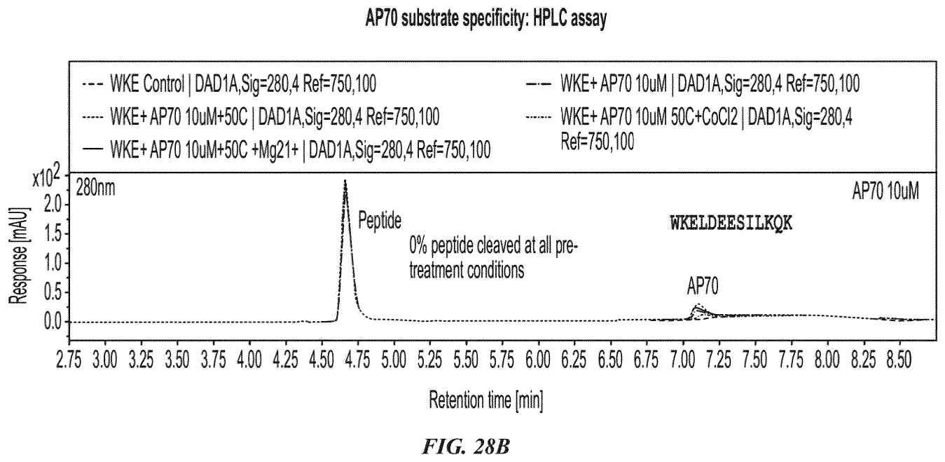

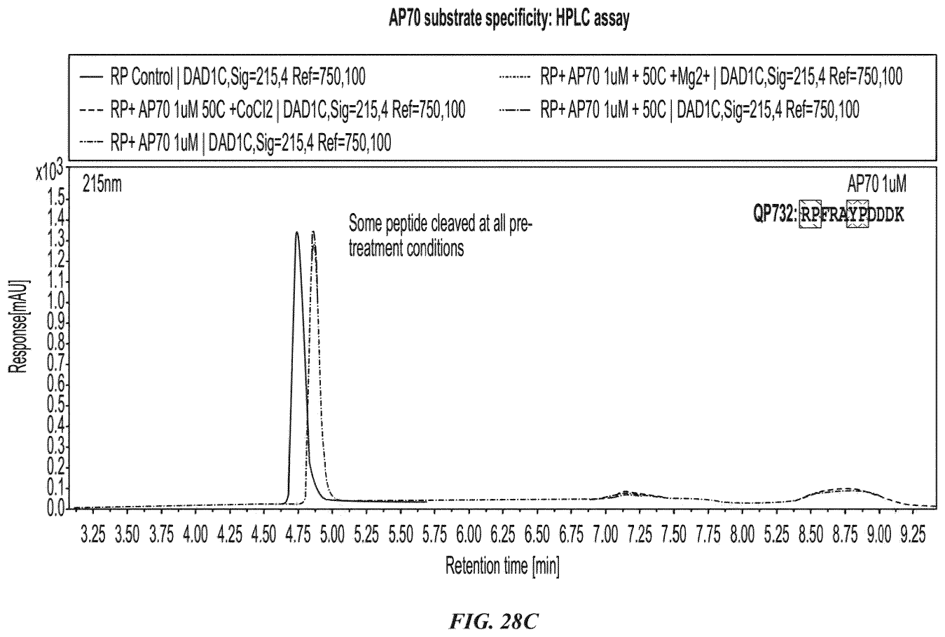

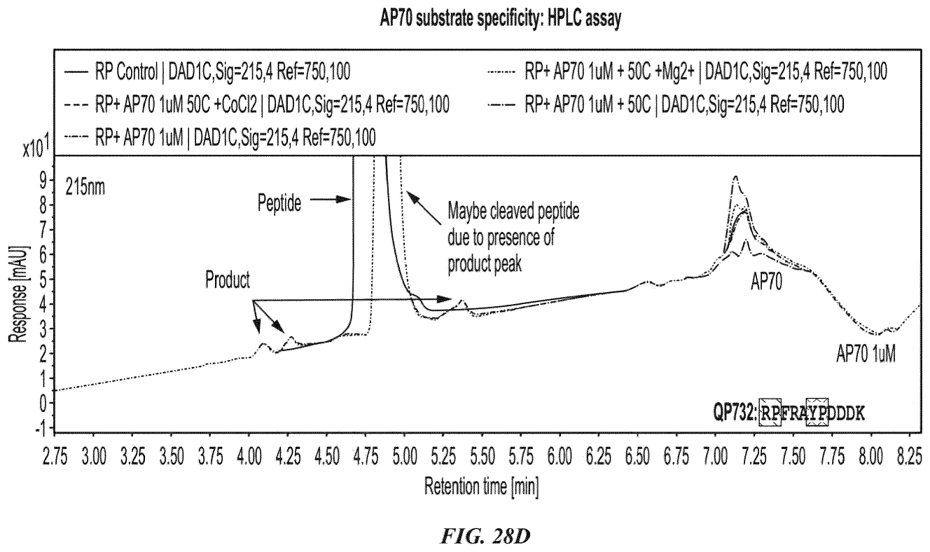

FIGS. 28A-28D show results from HPLC assay for different peptide cleavage by AP70 after different conditioning treatments and unconditioned control. AP70 Pre-treatment Conditions: No treatment; 50° C., 30 min; 50° C.+Co²⁺5 mM, 30 min; 50° C.+Mg²⁺5 mM, 30 min. HPLC Reaction Conditions: 200 μM Peptide; 30 min at 30° C.; Quench using Formic acid.

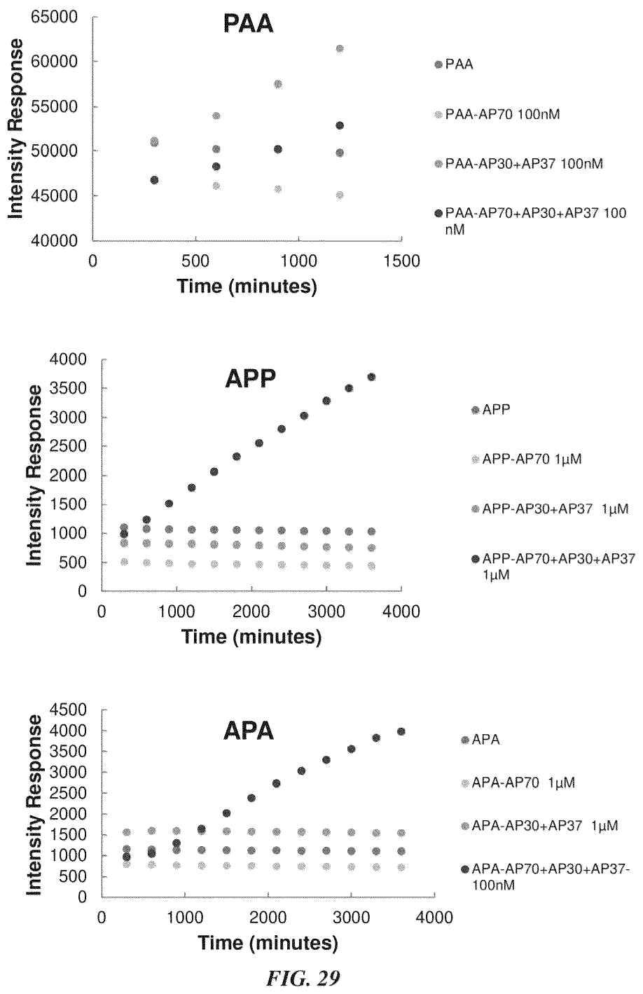

A real-time cleavage kinetics assay was performed using short peptide-AMC (7-Amino-4-methylcoumarin) substrates, with cleavage kinetics followed in real time at excitation wavelength of 357 nm and emission wavelength of 450 nm, at 30° C. to measure the increase in fluorescence at 441 nm. FIG. 29 shows three graphs comparing the cleavage activity and substrate specificities for AP70, AP30+AP37, and AP30+AP37+AP70 combinations on PXX, XPP, XPX peptides containing proline. The cleavage activities were derived for each peptide at optimal concentrations of aminopeptidases, which were determined by aminopeptidases titration.

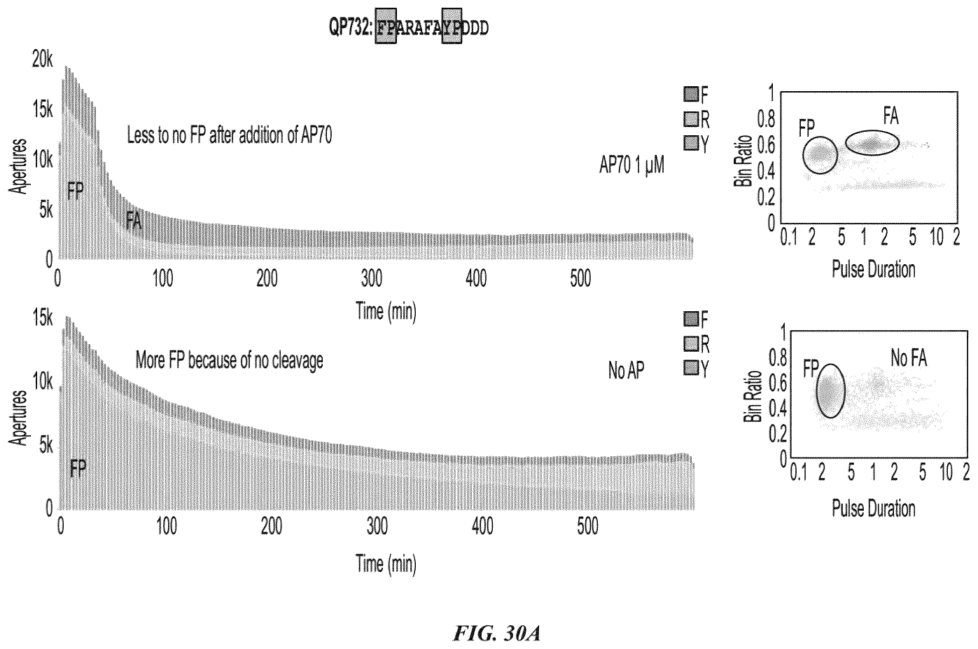

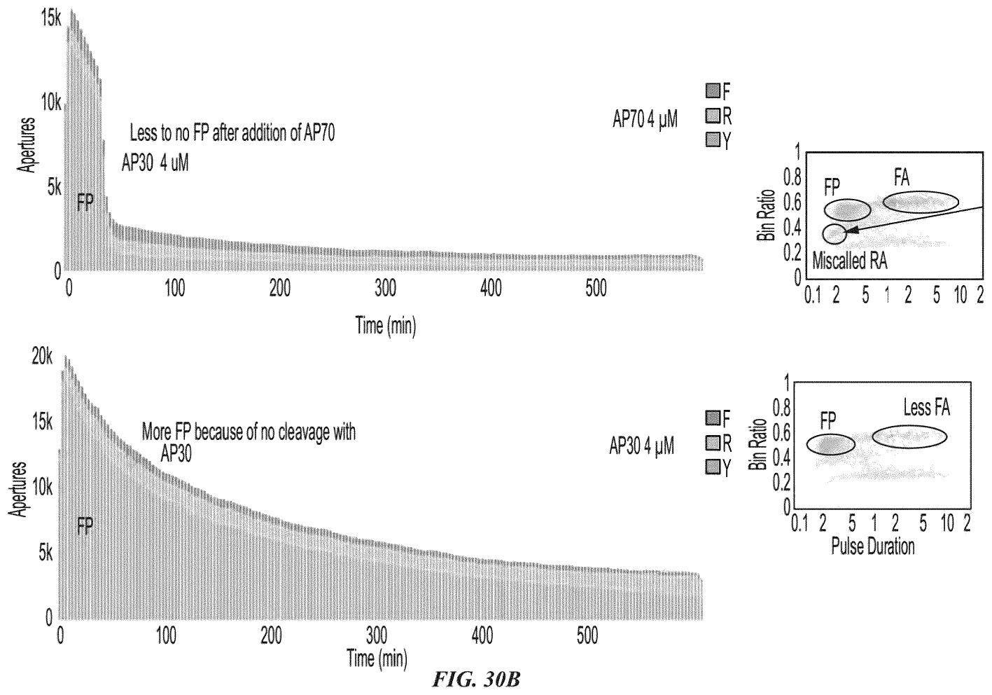

FIGS. 30A-30B show results from protein sequencing assay with AP70 (1 μM, 4 μM), no AP control, AP30 (4 μM) on QP734 (FPARAFAYPDDD) (SEQ ID NO: 52) peptide along with PS610 (50 nM)/PS961 (125 nM)/PS1122 (250 nM) recognizers. Apertures vs. Time plots display FP, RA and FA levels through the chip run (left panels). Bin Ratio vs. Pulse duration plots are shown with separated clusters for recognized RSs (right panels; arrow in third panel from top shows data which matches Bin ration of PS1122 but miscalled as FP).

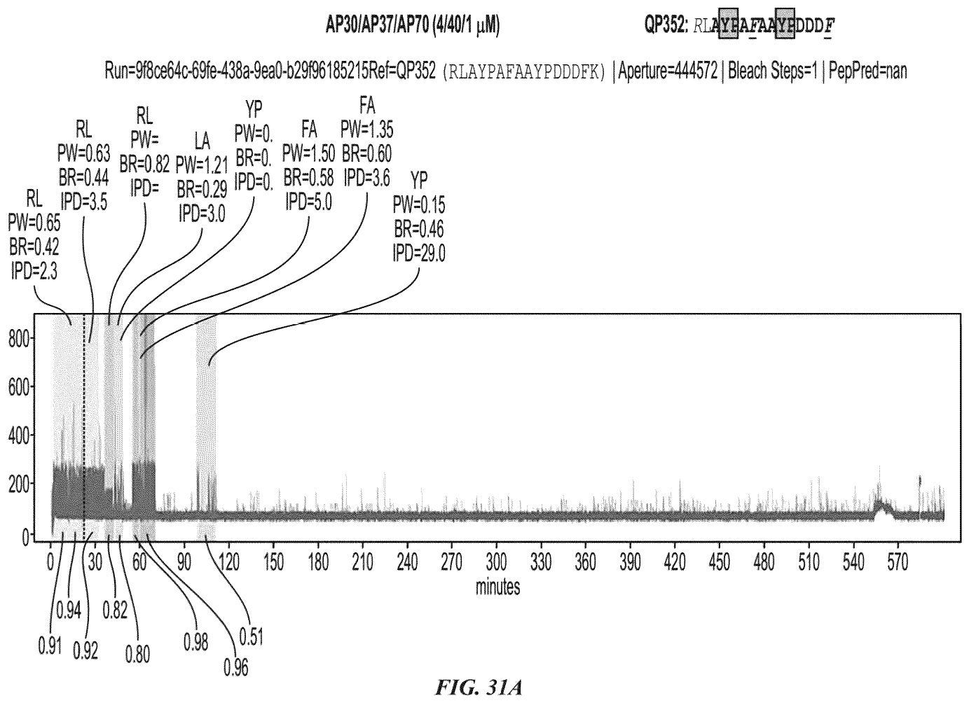

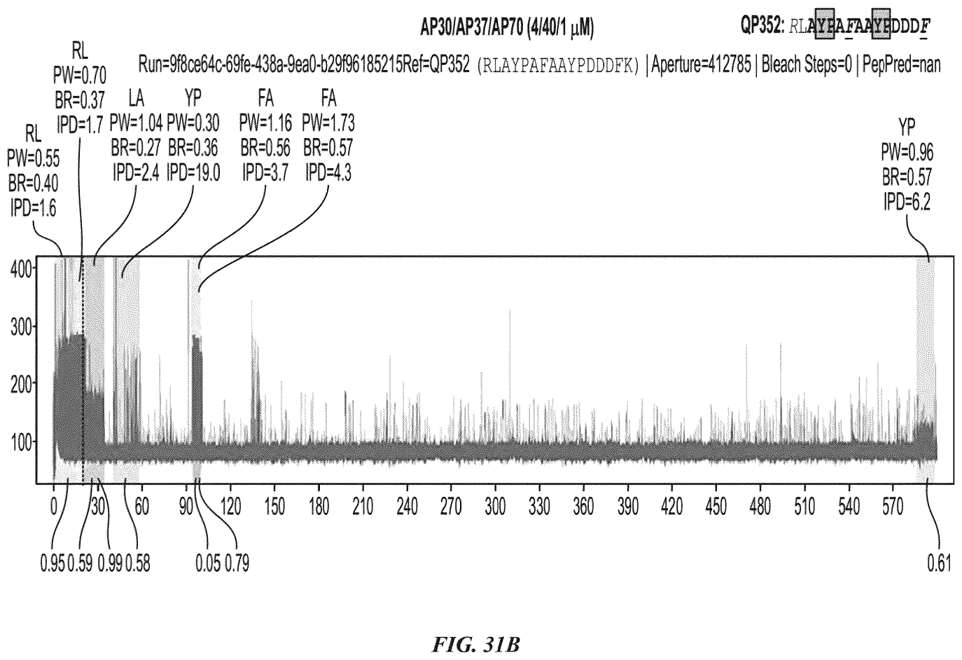

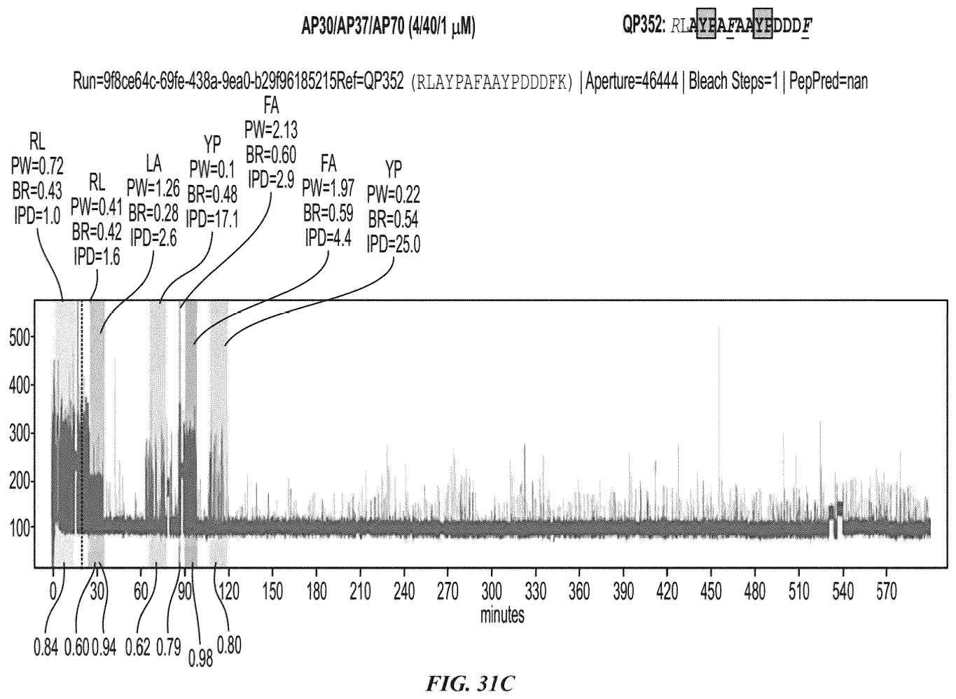

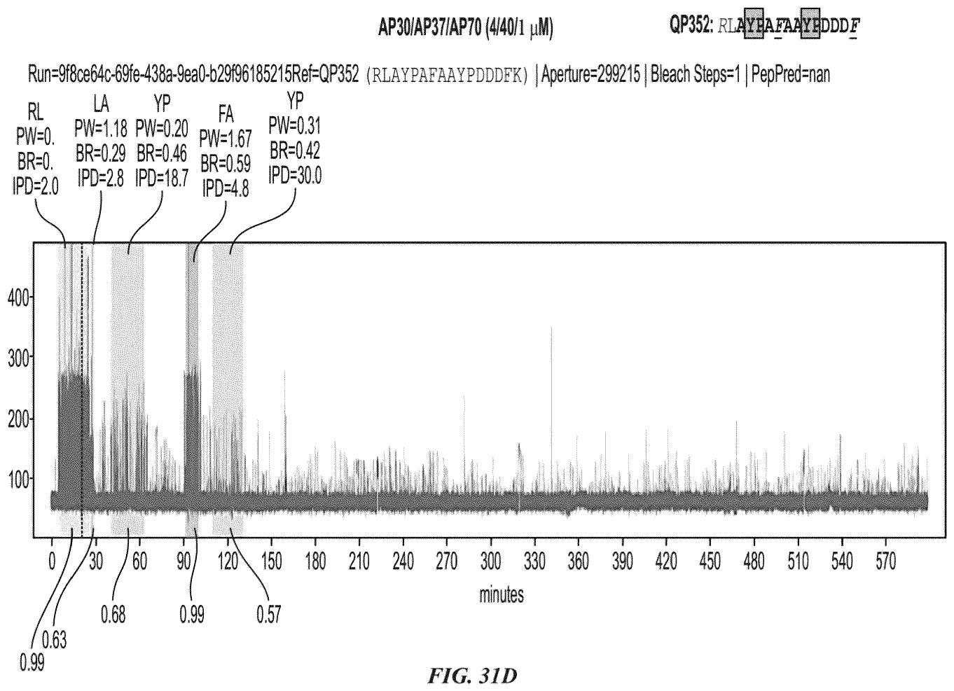

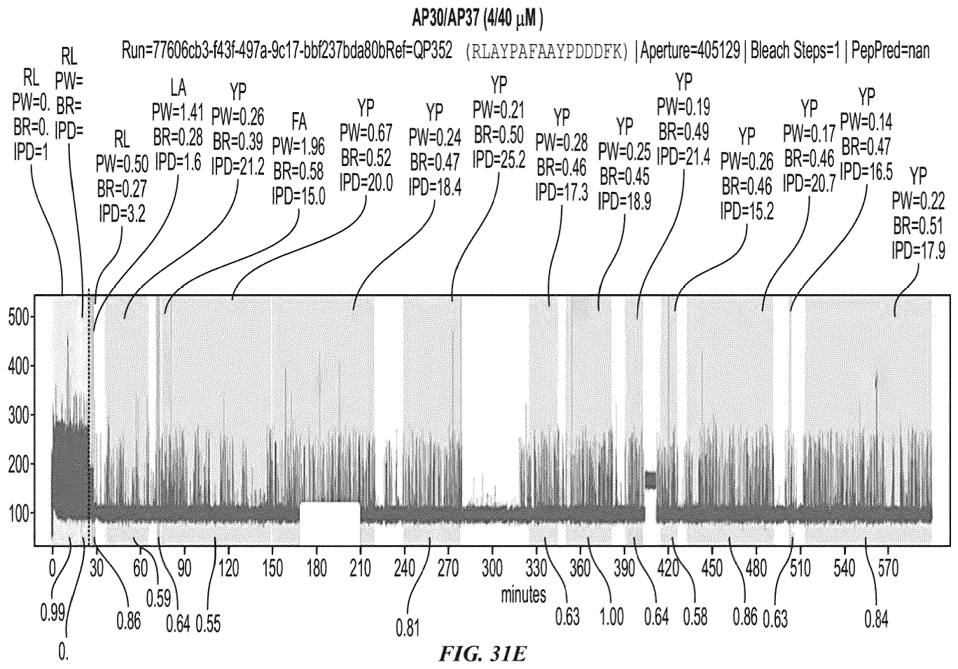

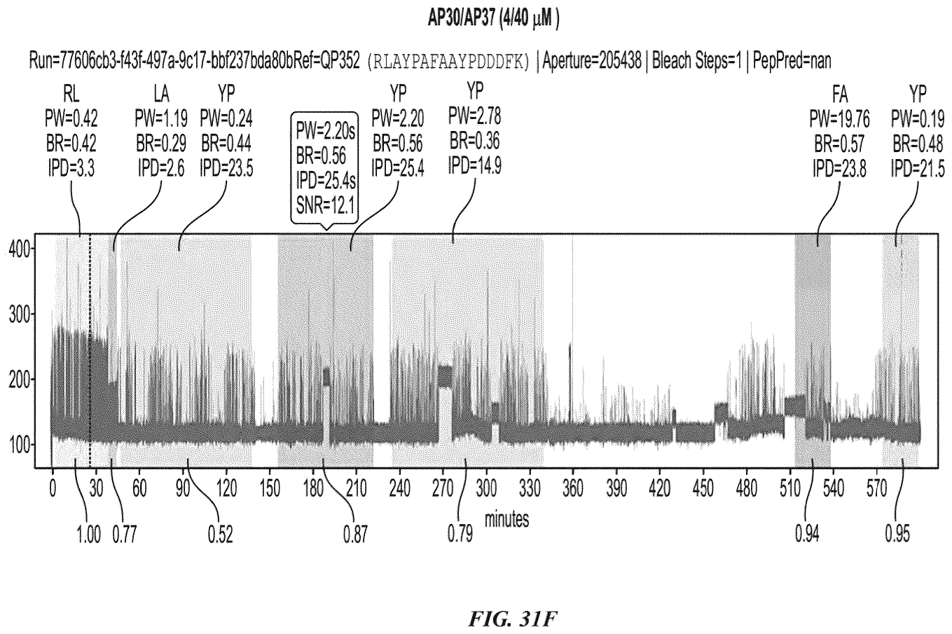

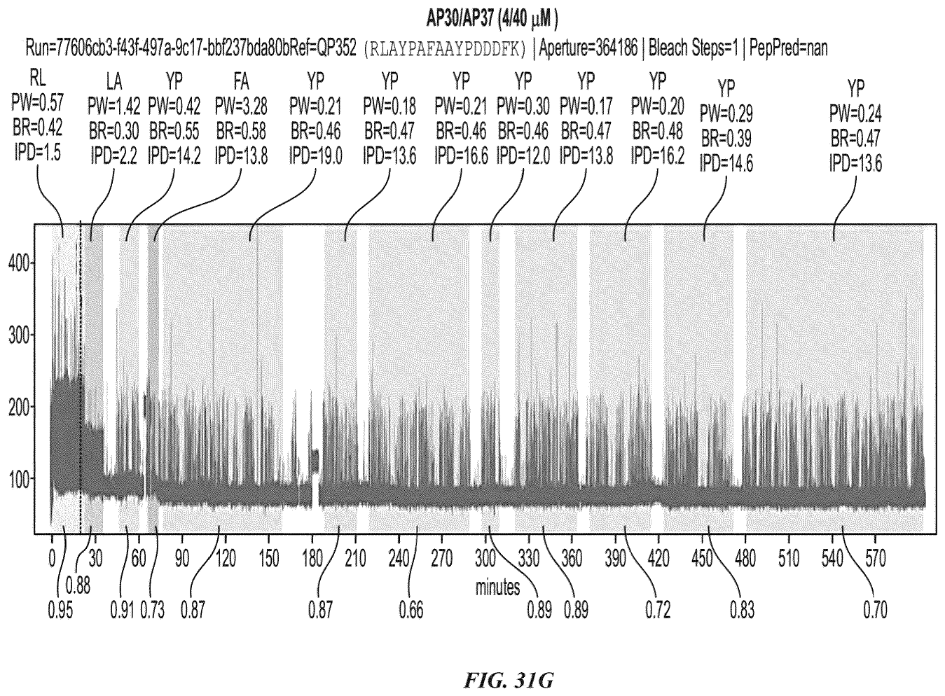

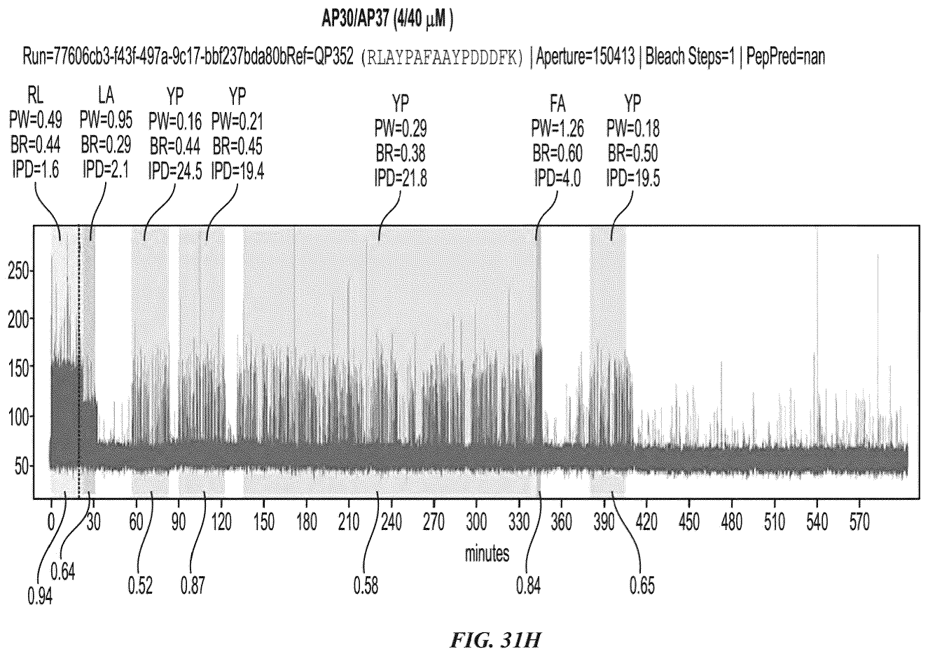

FIGS. 31A-31H show representative traces from protein sequencing assays showing the effect of adding AP70 (1 μm) in the AP30/AP37 (4/40 μM) aminopeptidase combination. The protein sequencing assay was carried out with (FIGS. 31A-31D) AP30 (1 μM)/AP37 (40 μM)/AP70 (1 μM), and (FIGS. 31E-31H) AP30 (1 μM)/AP37 (40 μM) combinations on QP352 (RLAYPAFAAYPDDDF) (SEQ ID NO: 51) peptide along with PS610 (50 nM)/PS961 (125 nM)/PS1122 (250 nM) recognizers. Table 6 shows results from AP70 concentration titration to optimize the AP30+AP37+AP70 AP combination performance on chips for QP352 (RLAYPAFAAYPDDDF) (SEQ ID NO: 51) sequencing.

TABLE 6
Results from AP70 concentration titration

AP70 with AP30/AP37 at 4/40 μM

| AP70 | | | | Overall |
|---|---|---|---|---|
| Concentration | % 1^{st} | % Last | | % FA |
| (μM) | YP* | YP* | % FA* | Reached¹ |
| 0.25 | 21.46 | 2.46 | 9.13 | 11.59 |
| 0.50 | 19.18 | 2.36 | 8.50 | 10.6 |
| 1 | 21.07 | 6.53 | 16.85 | 23.39 |
| 2 | 9.94 | 1.22 | 12.93 | 14.14 |
| 4 | 13.78 | 3.79 | 18.01 | 21.8 |
| 6 | 14.28 | 2.29 | 11.21 | 13.49 |

AP30/AP37 at 4/40 μM

| AP70 | | | | Overall |
|---|---|---|---|---|
| Concentration | % 1^{st} | % Last | | % FA |
| (μM) | YP* | YP* | % FA* | Reached¹ |
| 0.25 | | | | |
| 0.50 | | | | |
| 1 | 49.04 | 0.72 | 3.22 | 3.95 |
| 2 | | | | |
| 4 | 69.24 | 1.59 | 2.69 | 4.28 |
| 6 | | | | |

*Reads with 1 gap allowed;
¹Overall % FA reached >1 gaps allowed in reads

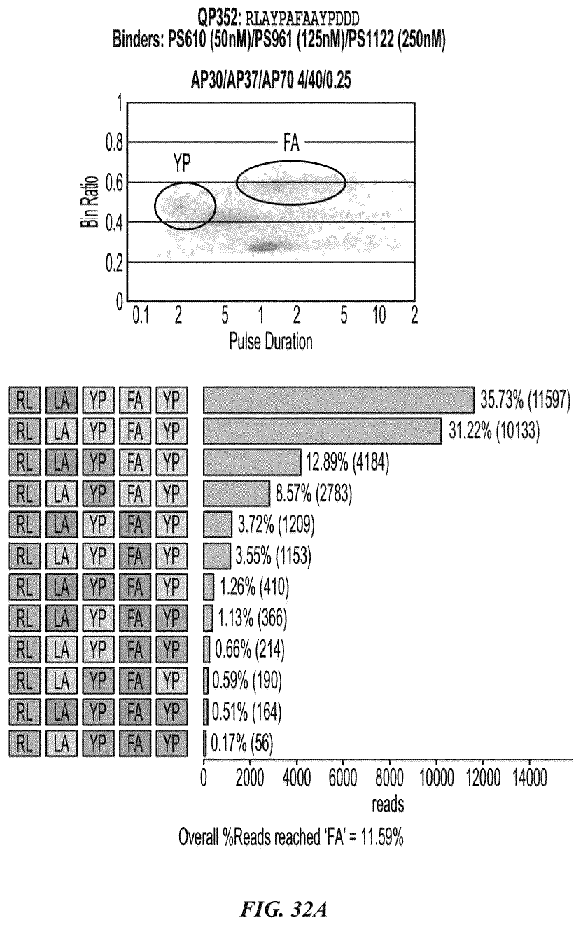

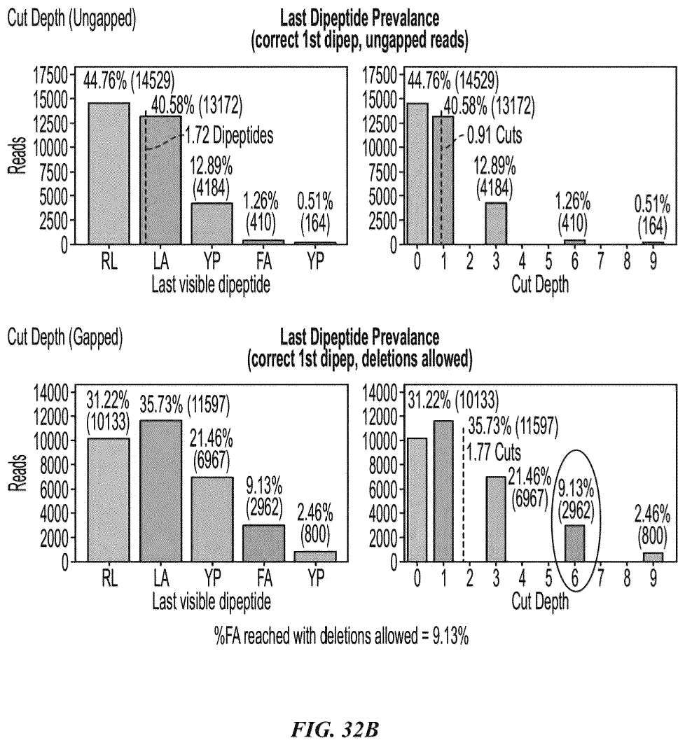

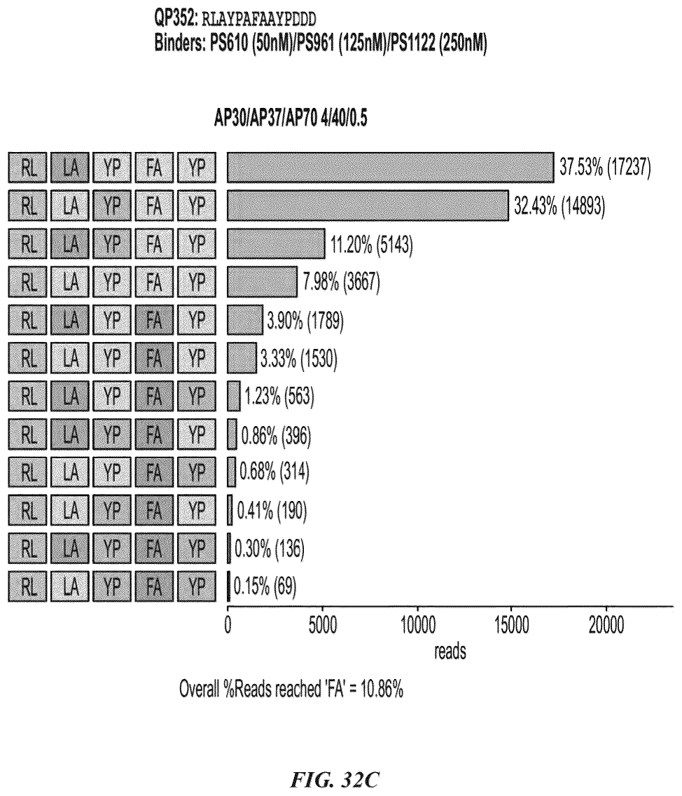

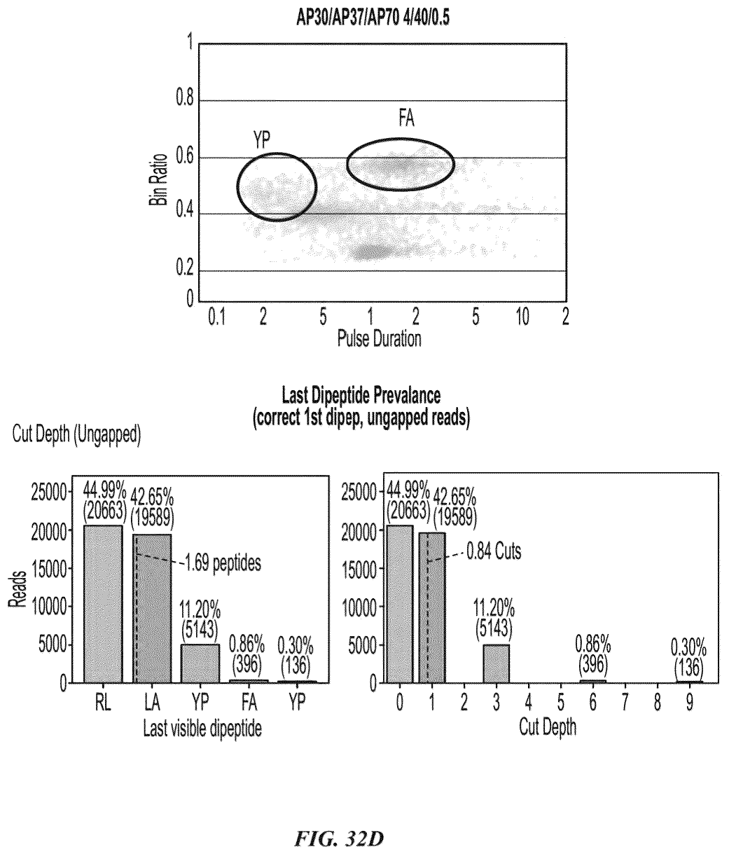

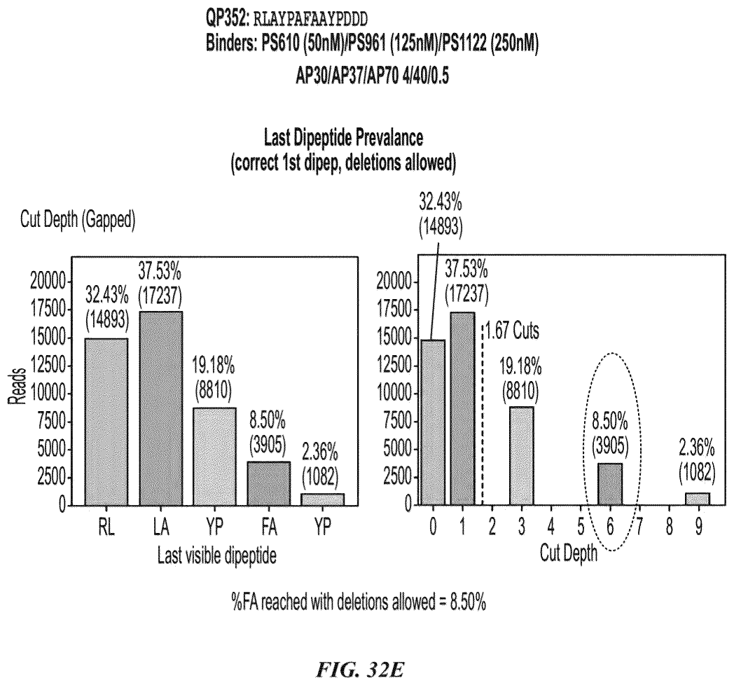

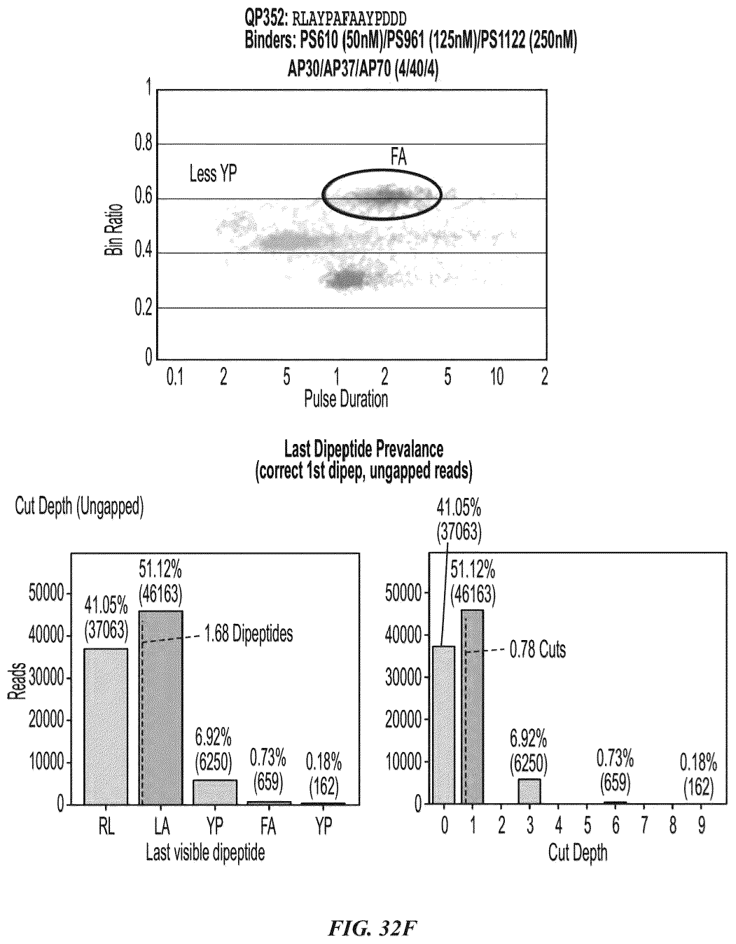

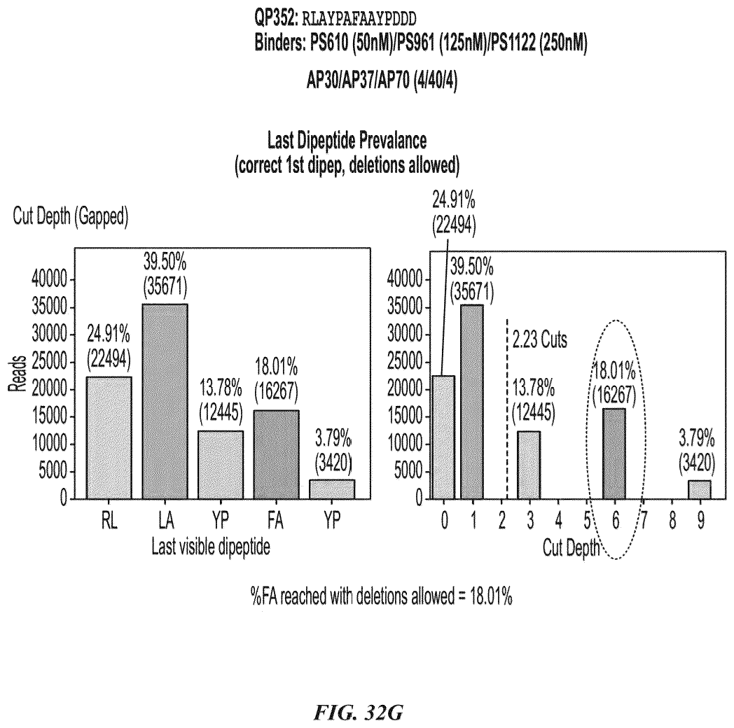

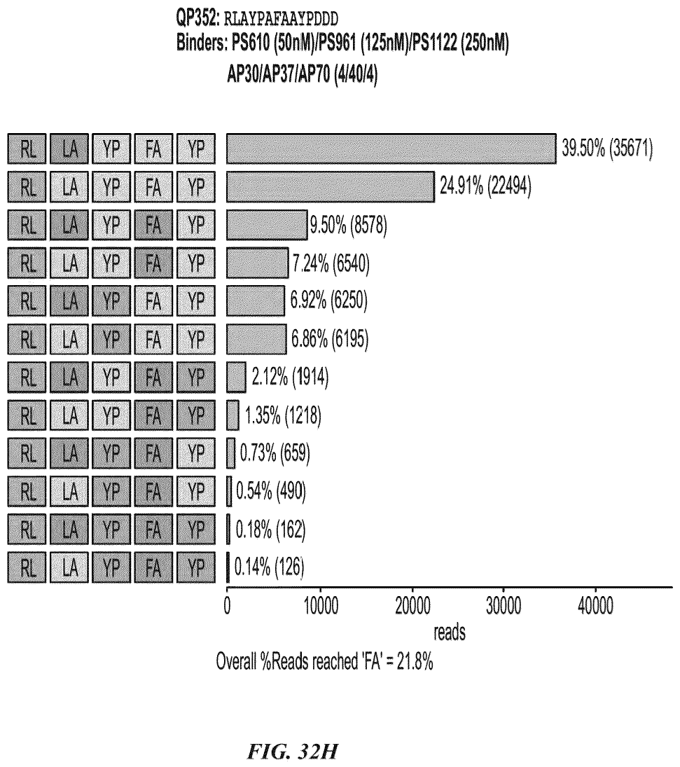

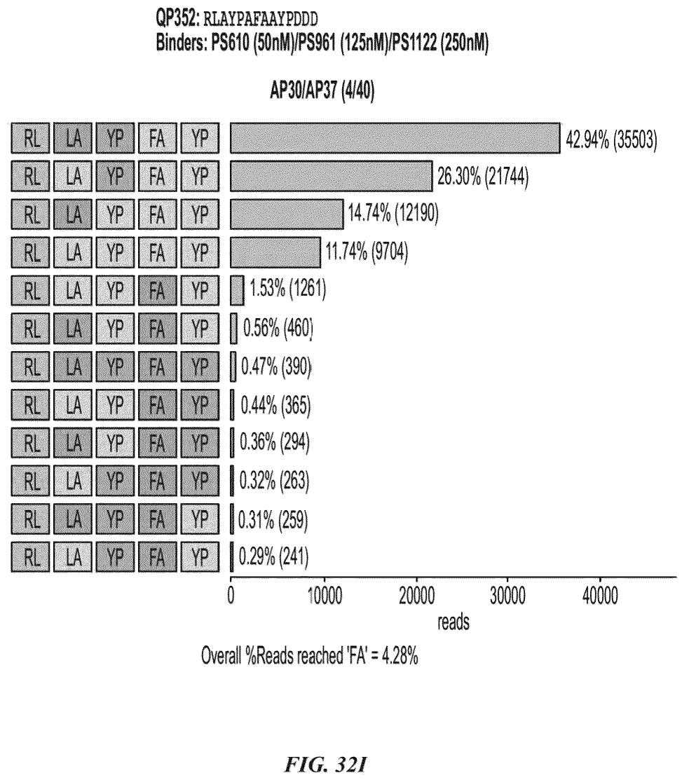

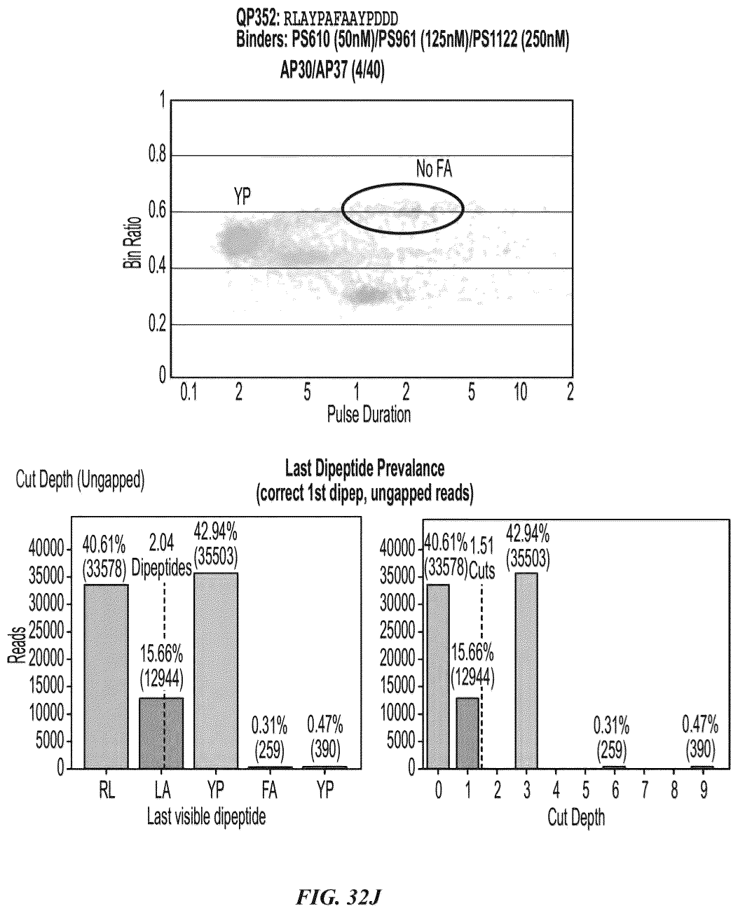

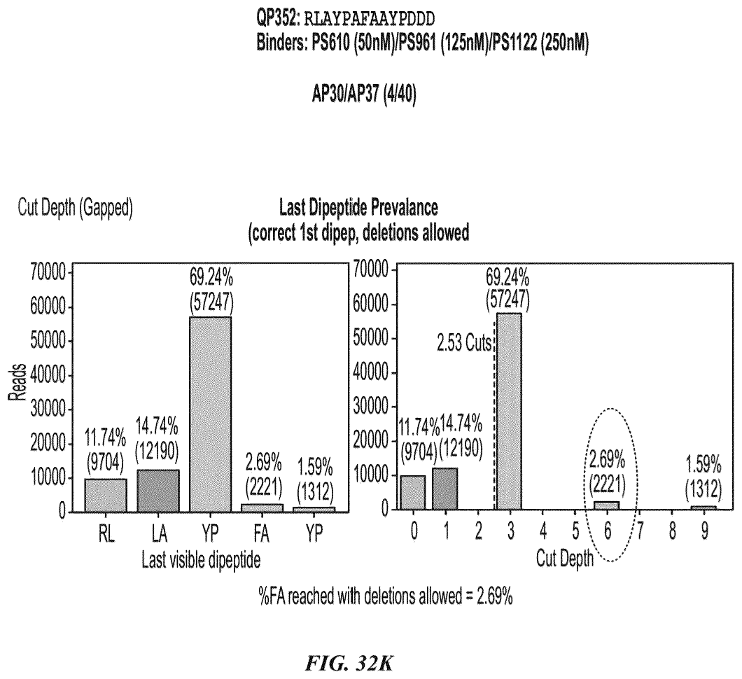

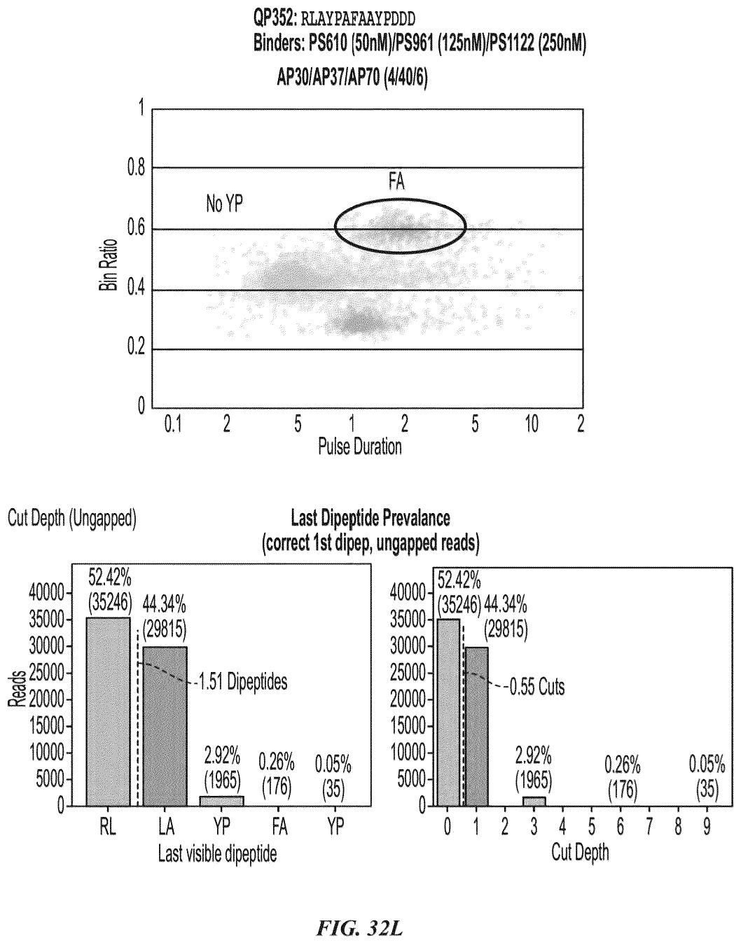

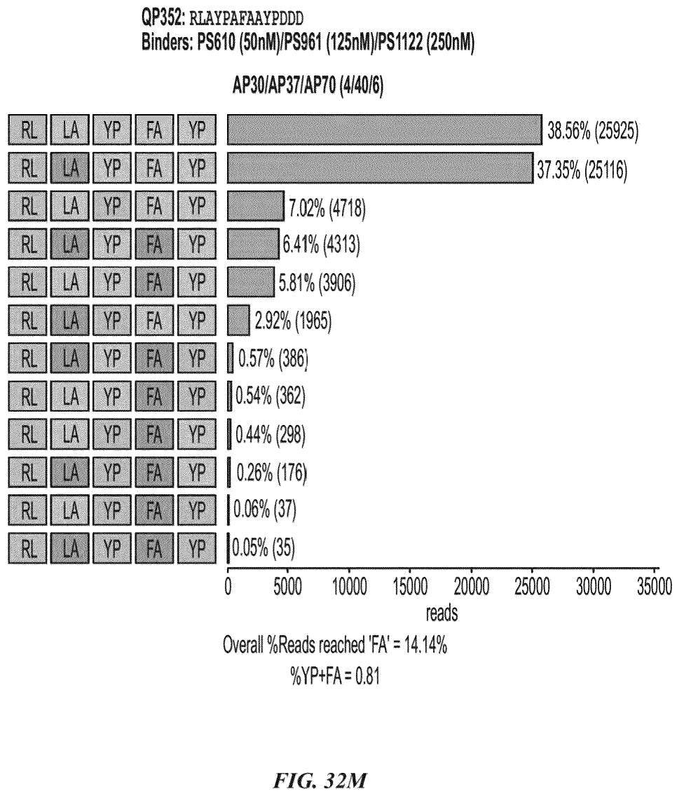

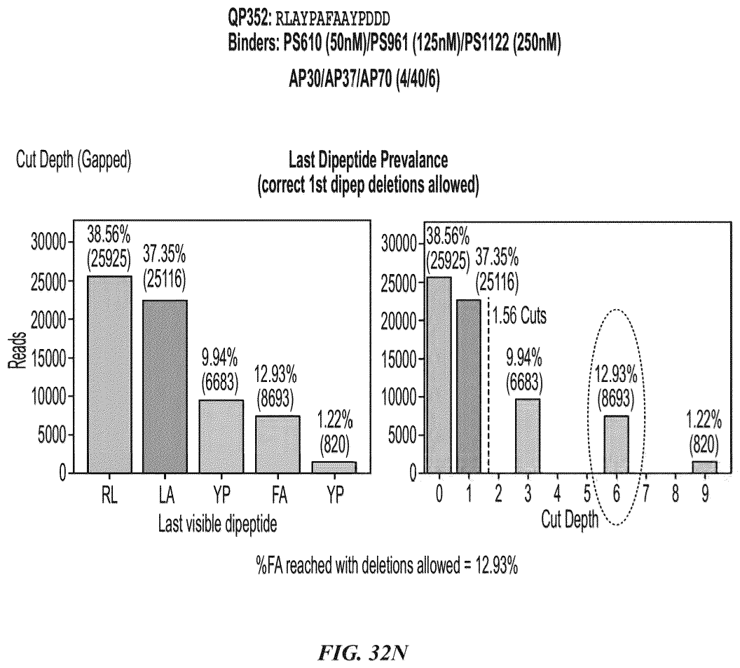

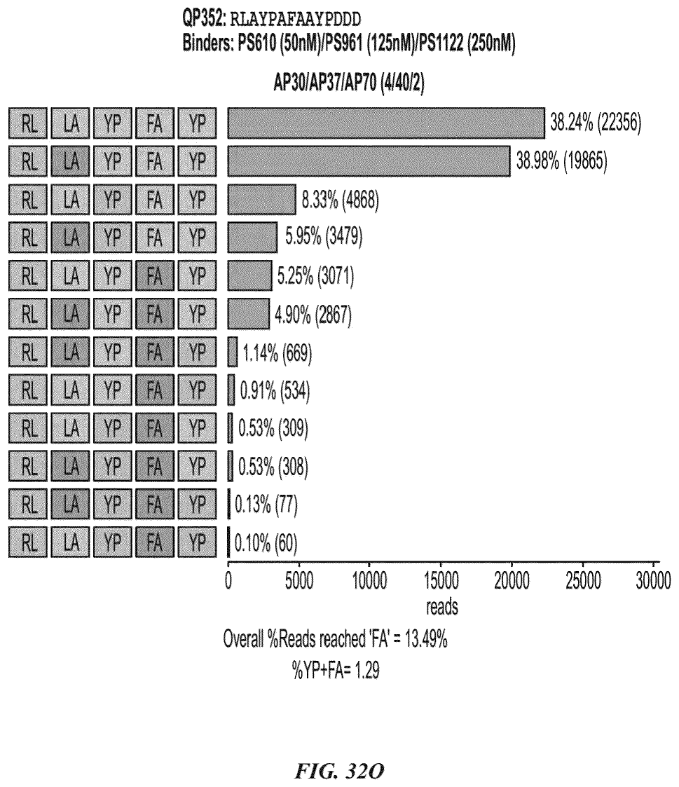

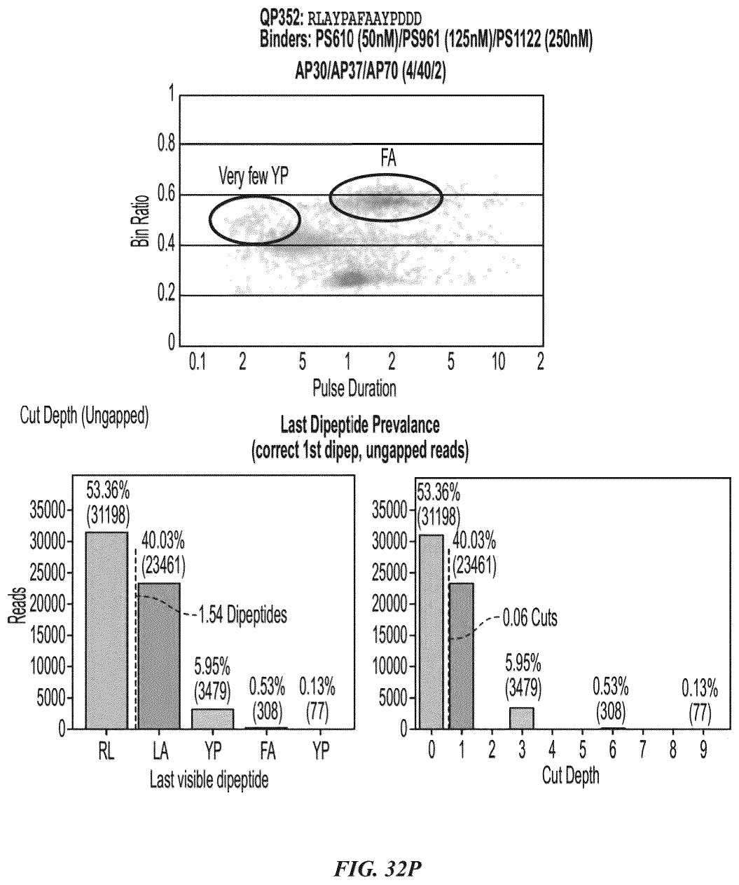

Results from additional runs performed to generate the data in Table 6 are shown in FIGS. 32A-32Q. The runs of FIGS. 32A-32B and 32C-32E showed a similar performance at both AP70 concentrations. The runs of FIGS. 32L-32N and 320-32Q showed: (a) a similar performance for both concentrations of AP70 in terms of reads reached FA, and (b) for AP70 at 6 μM, RSC of both 1^{st} and last YP increased, which was reflected in % YP reads being less as compared to AP70 at 2 μM.

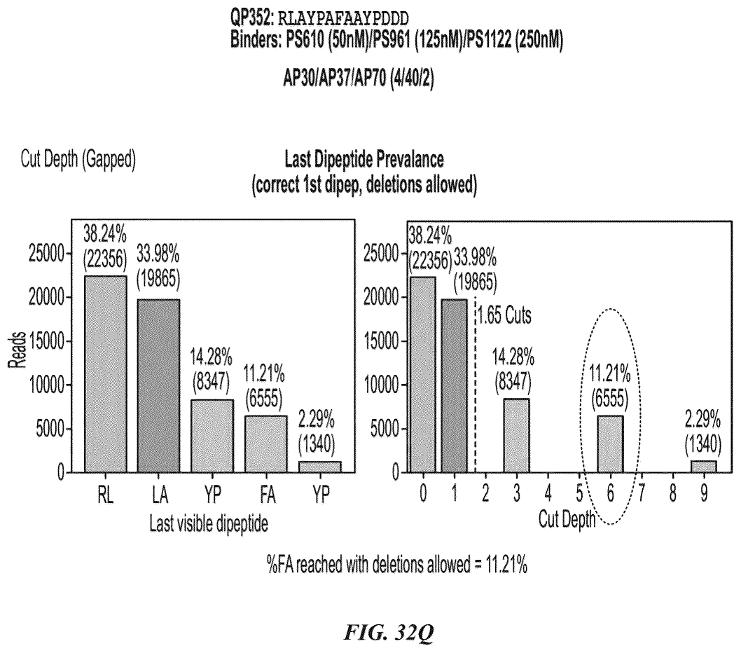

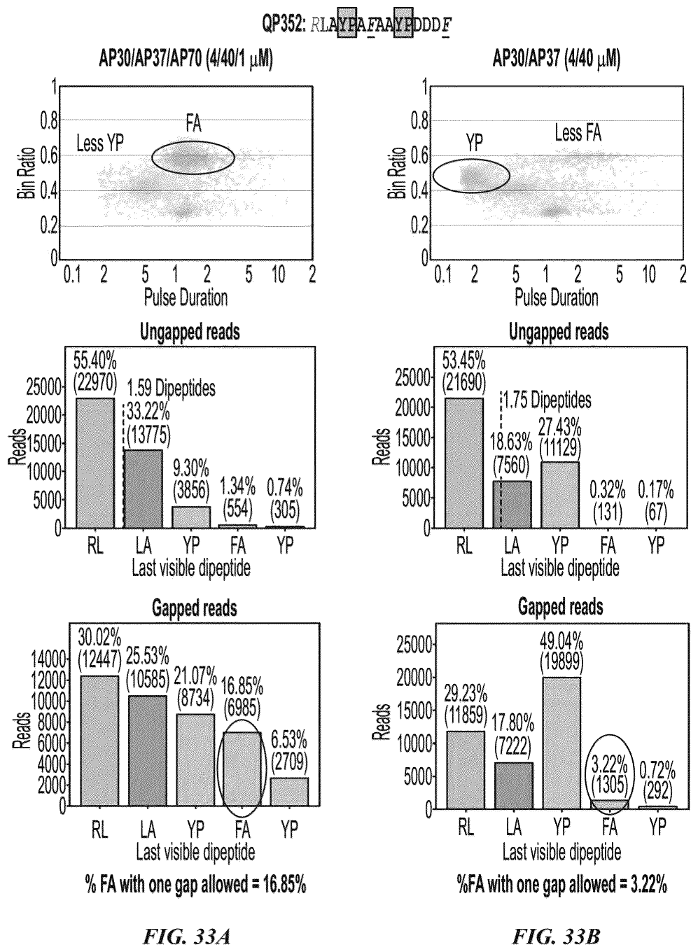

FIGS. 33A-33B show example results from protein sequencing assays with (FIG. 33A) AP30 (4 μM)/AP37 (40 μM)/AP70 (1 μM), and (FIG. 33B) AP30 (4 μM)/AP37 (40 μM) combinations on QP352 (RLAYPAFAAYPDDDF) (SEQ ID NO: 51) peptide along with PS610 (50 nM)/PS961 (125 nM)/PS1122 (250 nM) recognizers. Bin Ratio vs. Pulse duration plot with separated clusters for recognized RSs are shown in top panels. The presence of “FA” cluster with AP70 at 1 μM in combination with AP30/AP37 at 4/40 μM confirms that AP70 is able to cleave past the “Y-P” motif, thus increasing the cut depth. Also shown in FIG. 33 are bar plots displaying the cut depths and proportion of each RS recognized in the reads without gap (middle panels) and with gaps (bottom panels).

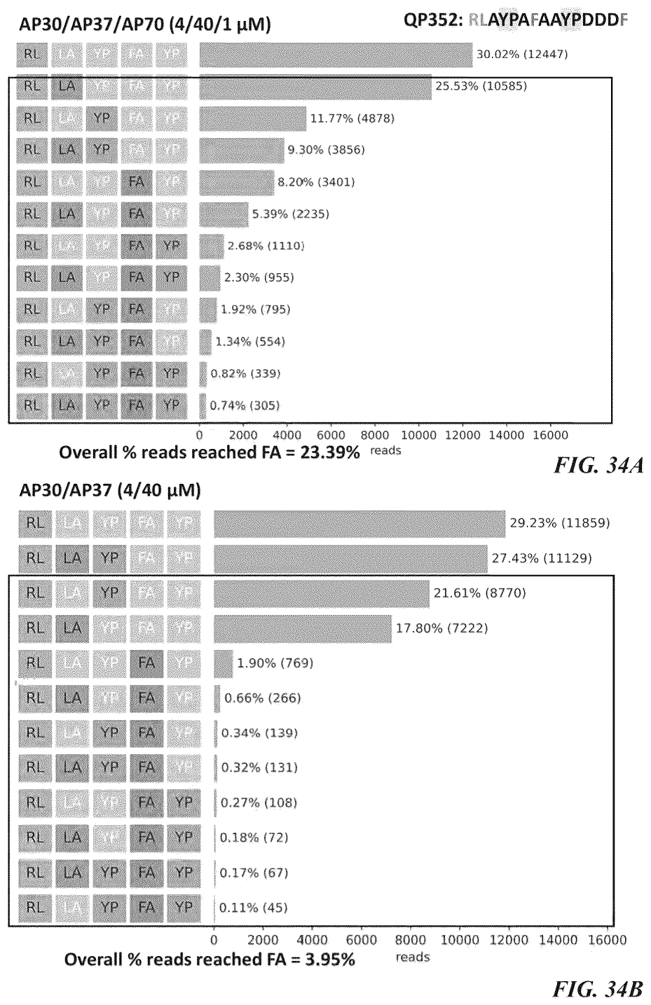

FIGS. 34A-34B show reads proportion breakdown for the QP352 (RLAYPAFAAYPDDDF) (SEQ ID NO: 51) protein sequencing assay with (FIG. 34A) AP30 (4 μM)/AP37 (40 μM)/AP70 (1 μM) and (FIG. 34B) AP30 (4 μM)/AP37 (40 μM) combinations along with PS610 (50 nM)/PS961 (125 nM)/PS1122 (250 nM) recognizers.

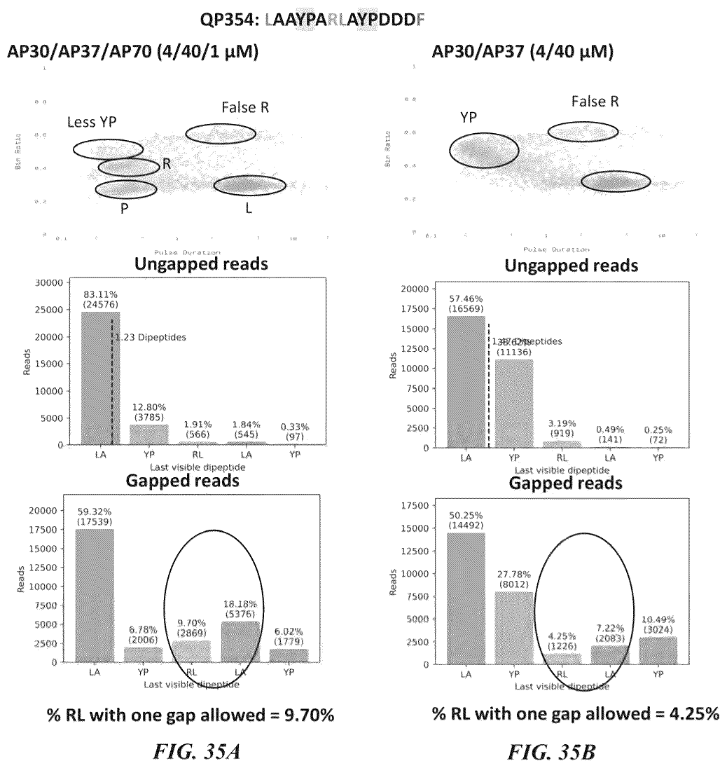

FIGS. 35A-35B show example results from protein sequencing assays with (FIG. 35A) AP30 (4 μM)/AP37 (40 μM)/AP70 (1 μM), and (FIG. 35B) AP30 (4 μM)/AP37 (40 μM) combinations on QP354 (LAAYPARLAYPDDDF) (SEQ ID NO: 53) peptide along with PS610 (50 nM)/PS961 (125 nM)/PS1122 (250 nM) recognizers. Bin Ratio vs. Pulse duration plot with separated clusters for recognized RSs are shown in top panels. Also shown are bar plots displaying the cut depths and proportion of each RS recognized in the reads without gap (middle panels) and with gaps (bottom panels). These results demonstrated that AP70 showed YP cleavage on an alternate peptide QP352.

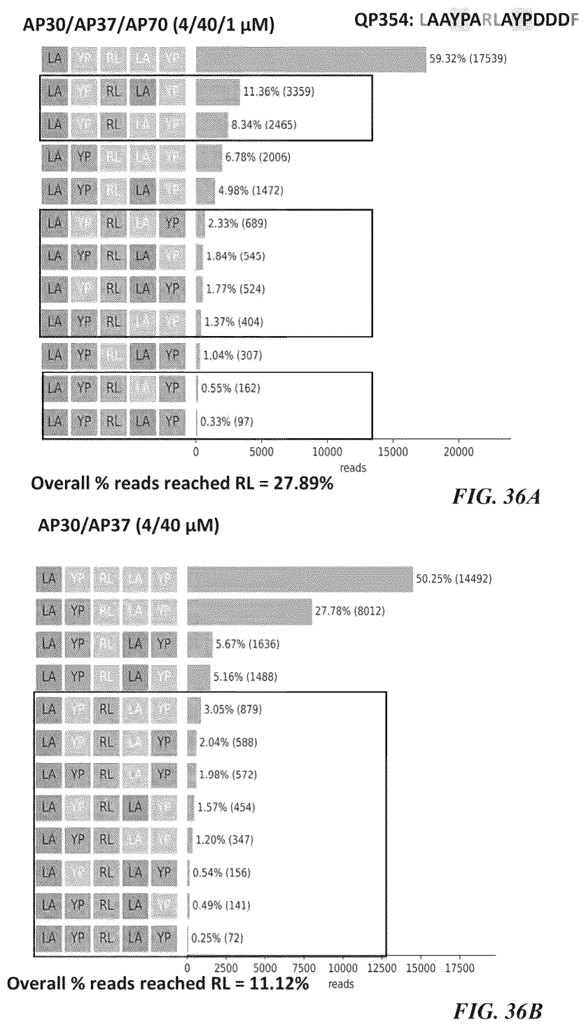

FIGS. 36A-36B show reads proportion breakdown for the QP354 (LAAYPARLAYPDDDF) (SEQ ID NO: 53) protein sequencing assay with (FIG. 36A) AP30 (4 μM)/AP37 (40 μM)/AP70 (1 μM), and (FIG. 36B) AP30 (4 μM)/AP37 (40 μM) combinations along with PS610 (50 nM)/PS961 (125 nM)/PS1122 (250 nM) recognizers. These results demonstrated that AP70 showed consistent performance on an alternate peptide of the same length as QP352.

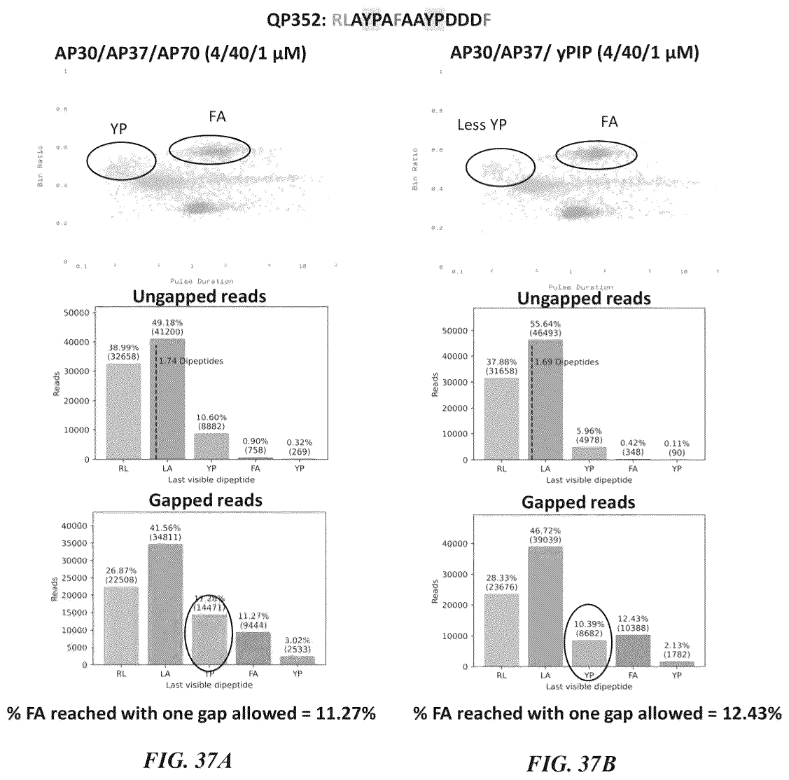

FIGS. 37A-37B show results from protein sequencing assays with (FIG. 37A) AP30 (4 μM)/AP37 (40 μM)/AP70 BC-B1 batch (1 μM), and (FIG. 37B) AP30 (4 μM)/AP37 (40 μM)/yPIP BC-B1 batch (1 μM) combinations on QP352 (RLAYPAFAAYPDDDF) (SEQ ID NO: 51) peptide along with PS610 (50 nM)/PS961 (125 nM)/PS1122 (250 nM) recognizers. Bin Ratio vs. Pulse duration plot with separated clusters for recognized RSs are shown in top panels. Also shown are bar plots displaying the cut depths and proportion of each RS recognized in the reads without gap (middle panels) and with gaps (bottom panels). AP70 showed less RSC for the 1^{st} YP and reached the last YP for both ungapped and gapped reads as compared to yPIP. Table 7 shows yPIP and AP70 performance comparison with AP37+AP70 AP combination for QP352 (RLAYPAFAAYPDDDF) (SEQ ID NO: 51) sequencing.

TABLE 7
yPIP and AP70 performance comparison

| | | | |
|---|---|---|---|
| | | | Overall |
| % 1^{st} | % Last | | % FA |
| YP* | YP* | % FA* | Reached¹ |

AP70 at 1 μM with AP30/AP37 at 4/40 μM

| | | | |
|---|---|---|---|
| 17.28² | 3.02 | 11.27 | 14.28 |

yPIP at 1 μM with AP30/AP37 at 4/40 μM

| | | | |
|---|---|---|---|
| 10.39 | 2.13 | 12.43 | 14.57 |

*Gapped reads with 1 deletion allowed in reads;
¹Overall % FA reached >1 deletion allowed in reads;
Less 1st YP deletion with AP70

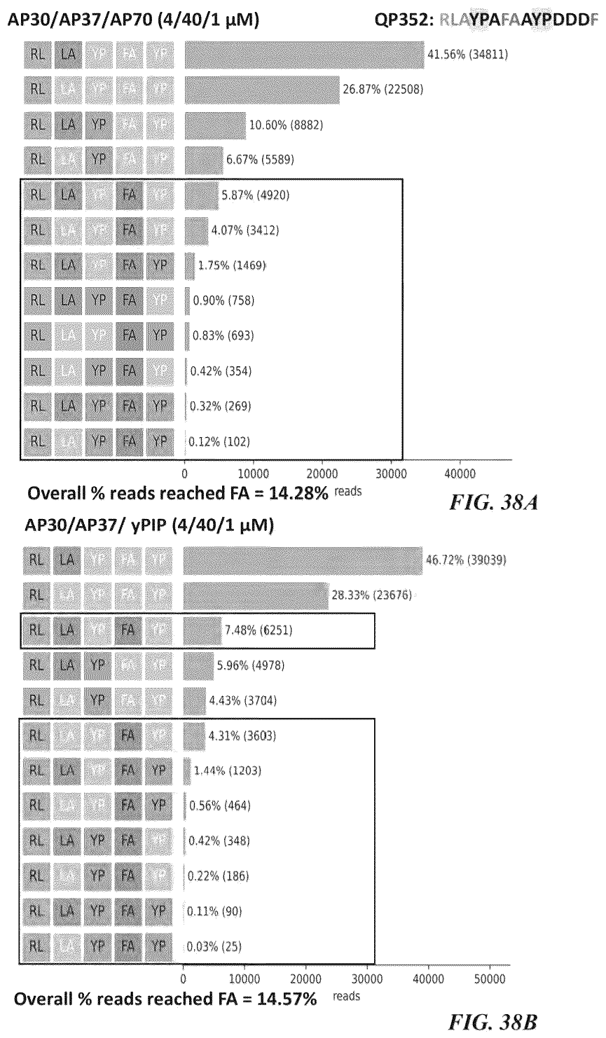

FIGS. 38A-38B show reads proportion breakdown for the QP352 (RLAYPAFAAYPDDDF) (SEQ ID NO: 51) protein sequencing assays with (FIG. 38A) AP30 (4 μM)/AP37 (40 μM)/AP70 BC-B1 batch (1 μM), and (FIG. 38B) AP30 (4 μM)/AP37 (40 μM)/yPIP BC-B1 batch (1 μM) combinations along with PS610 (50 nM)/PS961 (125 nM)/PS1122 (250 nM) recognizers. These results demonstrated that AP70 shows less RSC for the 1^{st} YP and reaches the last YP for both ungapped and gapped reads as compared to yPIP.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

1. A composition comprising:
a first cleaving reagent comprising a first aminopeptidase from Pyrococcus horikoshii and a first tag sequence; and
a second cleaving reagent comprising a second aminopeptidase from Pyrococcus horikoshii,
wherein the first and second aminopeptidases have different amino acid sequences.

2. The composition of claim 1, wherein the amino acid sequences of the first and second aminopeptidases share less than 80% sequence identity.

3. (canceled)

4. The composition of claim 1, wherein the first and second cleaving reagents are present in the composition at a first and second concentration, respectively, wherein the first concentration is at least two-fold higher than the second concentration.

5. The composition of claim 1, wherein the molar ratio of the first cleaving reagent to the second cleaving reagent in the composition is between about 2:1 and about 20:1.

6. (canceled)

7. The composition of claim 1, wherein the first cleaving reagent is present at a concentration of between about 10 μM and about 100 μM.

8. The composition of claim 1, wherein the first cleaving reagent is present in an amount sufficient to cleave an N-terminal amino acid from a polypeptide with an average cleavage time of between about 2 and about 60 minutes,
wherein the N-terminal amino acid comprises a charged side chain.

9. The composition of claim 1, wherein the second cleaving reagent is present at a concentration of between about 0.1 μM and about 25 μM.

10. The composition of claim 1, wherein the second cleaving reagent is present in an amount sufficient to cleave an N-terminal amino acid from a polypeptide with an average cleavage time of between about 2 and about 60 minutes,
wherein the N-terminal amino acid comprises a hydrophobic side chain.

11. (canceled)

12. The composition of claim 1, wherein the second cleaving reagent comprises a second tag sequence.

13-14. (canceled)

15. The composition of claim 12, wherein the first and second tag sequences each independently comprises between about 2 and about 200 amino acids.

16. The composition of claim 12, wherein at least one of the first and second tag sequences comprises a polyhistidine-tag and/or a biotinylation tag.

17-21. (canceled)

22. The composition of claim 1, wherein the first aminopeptidase has an amino acid sequence that is at least 80% identical to SEQ ID NO: 3.

23-25. (canceled)

26. The composition of claim 1, wherein the second aminopeptidase has an amino acid sequence that is at least 80% identical to SEQ ID NO: 1.

27-29. (canceled)

30. The composition of claim 1, further comprising:
a third cleaving reagent comprising an aminopeptidase from Yersinia pestis.

31. A composition comprising:
a first cleaving reagent comprising a first aminopeptidase having an amino acid sequence that is at least 80% identical to SEQ ID NO: 3;
a second cleaving reagent comprising a second aminopeptidase having an amino acid sequence that is at least 80% identical to SEQ ID NO: 1; and
a third cleaving reagent comprising a third aminopeptidase having an amino acid sequence that is at least 80% identical to SEQ ID NO: 5 or 7.

32-62. (canceled)

63. A reaction mixture for polypeptide analysis, the reaction mixture comprising:
a composition according to claim 1; and
one or more amino acid binding proteins not having peptide cleavage activity.

64. A reaction mixture for polypeptide analysis, the reaction mixture comprising:
a first cleaving reagent comprising a first aminopeptidase having an amino acid sequence that is at least 92% identical to SEQ ID NO: 3 and comprising a first tag sequence; and
one or more amino acid binding proteins not having peptide cleavage activity.

65. The reaction mixture of claim 64, further comprising:
a second cleaving reagent comprising a second aminopeptidase, wherein the amino acid sequences of the first and second aminopeptidases share less than 80% sequence identity.

66. A method of polypeptide analysis, the method comprising:
contacting a polypeptide with a reaction mixture according to claim 63;
monitoring a signal for signal pulses corresponding to interactions between one or more amino acid binding proteins and the polypeptide; and
determining at least one chemical characteristic of the polypeptide based on a characteristic pattern in the signal.

67. A method of polypeptide analysis, the method comprising:
contacting a polypeptide with a reaction mixture according to claim 64;
monitoring a signal for signal pulses corresponding to interactions between one or more amino acid binding proteins and the polypeptide; and
determining at least one chemical characteristic of the polypeptide based on a characteristic pattern in the signal.

68-72. (canceled)

73. A system comprising:
at least one hardware processor; and
at least one non-transitory computer-readable storage medium storing processor-executable instructions that, when executed by the at least one hardware processor, cause the at least one hardware processor to perform a method according to claim 66.

74. At least one non-transitory computer-readable storage medium storing processor-executable instructions that, when executed by at least one hardware processor, cause the at least one hardware processor to perform a method according to claim 66.
